(19) United States
(12) Patent Application Publication
(10) Pub. No.: US 2008/0069779 A1
(43) Pub. Date: Mar. 20, 2008

(54) Foamable vehicle and vitamin and flavonoid pharmaceutical compositions thereof

(75) Inventors: Dov Tamarkin, Maccabim (IL); Doron Friedman, Karmei Yosef (IL); Meir Eini, Ness Ziona (IL); Tal Berman, Rishon LeZiyyon (IL); David Schuz, Moshav Gimzu (IL)

Correspondence Address: WILMERHALE/BOSTON, 60 STATE STREET, BOSTON, MA, 02109, US

(73) Assignee: Foamix Ltd., Ness Ziona (IL)

(21) Appl. No.: 11/900,072

(22) Filed: Sep. 10, 2007

Related U.S. Application Data
(63) Continuation-in-part of application No. 11/430,599, filed on May 9, 2006.
(63) Continuation-in-part of application No. 10/835,505, filed on Apr. 28, 2004.
(60) Provisional application No. 60/843,140, filed on Sep. 8, 2006.
(60) Provisional application No. 60/784,793, filed on Mar. 21, 2006.
(60) Provisional application No. 60/679,020, filed on May 9, 2005.
(60) Provisional application No. 60/530,015, filed on Dec. 16, 2003.
(60) Provisional application No. 60/492,385, filed on Aug. 4, 2003.

(51) Int. Cl. A61K 9/12; A61P 37/00

(52) U.S. Cl. 424/45

(57) ABSTRACT

Vitamin and flavonoid containing compositions are provided that are stable to degradation. Stabilized compositions include one or more features including a hygroscopic solvent at a sufficient concentration to provide an Aw value of the hygroscopic vitamin and or flavonoid containing composition of less than 0.9, antioxidant flavonoids that are preferentially oxidized before the vitamin, preservatives, and hydrocarbon propellants selected to reduce the oxidation potential of the composition.

### CROSS REFERENCE TO RELATED APPLICATIONS

This application claims the benefit under 35 U.S.C. §119(e) of U.S. Provisional Patent Application No. 60/843,140, filed on Sep. 8, 2006, and U.S. Provisional Patent Application No. TBA, filed on Sep. 4, 2007, each of which is entitled “Foamable Vehicle and Vitamin Pharmaceutical Compositions Thereof.” This application is also a continuation-in-part application of co-pending U.S. patent application Ser. No. 11/430,599 filed on May 9, 2006, which claims the benefit under 35 U.S.C. §119(e) of U.S. Provisional Patent Application No. 60/679,020, filed on May 9, 2005, entitled Hygroscopic Anti-Infective Compositions, and U.S. Provisional Patent Application No. 60/784,793, filed on Mar. 21, 2006, entitled Polyol Foamable Vehicle and Pharmaceutical Compositions Thereof, and which is also a continuation-in-part application of co-pending U.S. patent application Ser. No. 10/835,505, filed on Apr. 28, 2004, which claims the benefit of priority under 35 U.S.C. §119(e) to U.S. Provisional Patent Application No. 60/530,015, filed on Dec. 16, 2003, and U.S. Provisional Patent Application No. 60/492,385, filed on Aug. 4, 2003, all entitled “Oleaginous Pharmaceutical Foam;” all of which are hereby incorporated in their entirety by reference.

### BACKGROUND

Vitamins are an essential component of the bodies' daily requirements and they are needed in various metabolic pathways and to maintain good health. Some vitamins are believed to support, facilitate, improve or optimize the function and effect of other vitamins and or active agents. Compositions which can be applied to the skin, a body cavity, a mucosal surface, the nose, the mouth, the eye, the ear canal, the respiratory system, the vagina and the rectum that contain an effective amount of one or more vitamins would be useful. Some vitamins are susceptible to oxidation and breakdown in solution. Vitamins may for example breakdown in the presence of water and may not be stable in compositions for sufficiently long periods of time to facilitate satisfactorily cosmetic and pharmaceutical uses. Vitamin absorption is on the other hand also retarded by substances like paraffin's and by certain pharmaceuticals.

External topical administration is an important route for the administration of drugs in disease treatment. Many groups of drugs, including, for example, antibiotic, anti-fungal, anti-inflammatory, anesthetic, analgesic, anti-allergic, corticosteroid, retinoid and anti-proliferative medications are preferably administered in hydrophobic media, namely ointment. However, ointments often form an impermeable barrier, so that metabolic products and excreta from the wounds to which they are applied are not easily removed or drained away. Furthermore, it is difficult for the active drug dissolved in the carrier to pass through the white petrolatum barrier layer into the wound tissue, so the efficacy of the drug is reduced. In addition, ointments and creams often do not create an environment for promoting respiration of the wound tissue and it is not favorable to the normal respiration of the skin. An additional disadvantage of petroleum jelly-based products relates to the greasy feeling left following their topical application onto the skin, mucosal membranes and wounds.

Foams and, in particular, foams that are substantially based on non-aqueous solvents are complicated systems which do not form under all circumstances. Whilst aqueous foam formulations are better known, they are also complicated and similarly do not form under all circumstances.

Antioxidants can be protective. Flavonoids are said to have high antioxidant activity and many health promoting effects, including anti-inflammatory and anti-allergic effects as well as reducing the risk of cancer and pulmonary disease, improved cardiovascular health and prevention of damage from free radicals. The use of flavonoids for protecting ascorbic acid and ascorbyl compounds has been proposed in respect of lotions creams and gels

By using specialized delivery systems like microsponges it may be possible to incorporate hydrophobic active ingredients within the microsponges, which active ingredients are released when the microsponges come into mechanical contact with the skin.

### SUMMARY

Compositions containing vitamins and/or flavonoids or derivative thereof using non-aqueous and aqueous carriers and foamable compositions containing them and methods of treatment are described.

Hygroscopic and aqueous stable vitamin containing compositions and foamable therapeutic vitamin compositions with and without additional active agents may be used for treatment of dermal and mucosal tissues are stable. They are non irritating, facilitate penetration at a target, are presentable in an easily applicable stable form, can be handled with ease thereby facilitating compliance and either minimize or eliminate the amount of free water and in consequence, the potential breakdown of vitamin by oxidation/hydrolysis, or provide an aqueous environment where they can resist or are protected from said potential breakdown.

In one or more embodiments, topical and body cavity foam formulations containing flavonoids on their own and in combination with one or more vitamins are described.

In one or more embodiments, topical and body cavity foam formulations containing microsponges comprising one or more active ingredients are provided. In particular, the microsponges comprise one or more vitamins or one or more flavonoids alone or in combination and also with other active ingredients.

In other embodiments, the use of flavonoids on their own, synergistically, and in combination with other active agents is disclosed.

In one or more aspects there is provided a hygroscopic vitamin and or flavonoid containing composition comprising:

a. at least one agent selected from the group consisting of vitamins and flavonoids;

b. at least one hygroscopic solvent at a sufficient concentration to provide an Aw value of the hygroscopic vitamin and or flavonoid containing composition of less than 0.9;

c. a foam stabilizer selected from the group consisting of at least one surface-active agent; at least one polymeric agent selected from the group consisting of a bioadhesive agent, a gelling agent, a film forming agent and a phase change agent and mixtures thereof; and

d. a propellant at a concentration of about 3% to about 25% by weight of the total composition,

wherein the composition is contained in a pressurized container, is substantially flowable and provides a foam upon release.

In one or more aspects there is provided a foamable hygroscopic vitamin and or flavonoid containing composition comprising:
a. at least one agent selected from the group consisting of vitamins and flavonoids;
b. at least one hygroscopic solvent at a sufficient concentration to provide an Aw value of the hygroscopic vitamin and or flavonoid containing composition of less than 0.9;
c. a foam stabilizer selected from the group consisting of at least one surface-active agent; and at least one polymeric agent selected from the group consisting of a bioadhesive agent, a gelling agent, a film forming agent and a phase change agent and mixtures thereof;
d. a hydrocarbon propellant at a concentration of about 3% to about 25% by weight of the total composition; and

wherein the composition is contained in a pressurized container to which a vacuum was applied prior to filling with a hydrocarbon propellant, is substantially flowable and provides a foam upon release, and

wherein applying a vacuum to and filling a canister containing the composition with hydrocarbon propellant contributes to stabilize the agent against degradation.

In one or more aspects, the hygroscopic vitamin and or flavonoid containing composition optionally may further comprise:

i. about 50% to about 98% of a polar solvent;

ii. 0% to about 48% of a secondary polar solvent;

iii. a foam adjuvant;

iv. 0% to about 5% by weight of at least one polymeric agent; and

v. 0% to about 5% of a conservation agent.

In one aspect, a hygroscopic vitamin and or flavonoid containing composition including at least one hygroscopic substance at a sufficient concentration to provide an Aw value of the hygroscopic pharmaceutical composition of less than 0.9; or the Aw value is in the range of about 0.8 and about 0.9; (2) about 0.7 and about 0.8; and (3) less than about 0.7 and a vitamin or a derivative thereof or a combinations thereof. The composition may be substantially free of water.

In one or more embodiments, the hygroscopic pharmaceutical composition further includes at least one component, selected from the group consisting of about 0.01% to about 5% by weight of at least one polymeric agent selected from a bioadhesive agent, a gelling agent, a film forming agent and a phase change agent; and a surface-active agent.

In one or more embodiments, the hygroscopic substance is selected from the group consisting of polyethylene glycols (PEGs), surfactants comprising PEG, polyols, monosaccharides, disaccharides, oligosaccharides and sugar alcohols in an amount to provide hygroscopic properties, and honey.

In one or more embodiments there is provided a foamable composition comprising a stabilized-vitamin, comprising:

a water-unstable vitamin;

an optional flavonoid; and

a foamable carrier comprising:
i. water;
ii. a foam stabilizer selected from the group consisting of at least one surface-active agent; at least one polymeric agent selected from the group consisting of a bioadhesive agent, a gelling agent, a film forming agent and a phase change agent and mixtures thereof;
iii. about 0.005% to about 5% conservational agent; and
iv. a propellant at a concentration of about 3% to about 25% by weight of the total composition,

where said foamable carrier reduces the destabilizing effect of water on the vitamin, and

wherein the composition is contained in a pressurized container, is substantially flowable and provides a foam upon release.

In one or more embodiments there is provided a method of treating a disorder or condition of mammalian subject, comprising:

administering a foam produced from a foamable vitamin and or flavonoid composition to a target site, foamable composition comprising a stabilized-vitamin, comprising:
a water-unstable vitamin;
an optional flavonoid; and
a foamable carrier comprising:
   i. water;
   ii. a foam stabilizer selected from the group consisting of at least one surface-active agent; at least one polymeric agent selected from the group consisting of a bioadhesive agent, a gelling agent, a film forming agent and a phase change agent and mixtures thereof;
   iii. about 0.005% to about 5% conservational agent; and
   iv. a propellant at a concentration of about 3% to about 25% by weight of the total composition,

where said foamable carrier reduces the destabilizing effect of water on the vitamin, and

wherein the composition is contained in a pressurized container, is substantially flowable and provides a foam upon release.

In one or more embodiments, the vitamin or flavonoid or combinations thereof are loaded into microsponges.

In one or more embodiments there is provided a method of treating a disorder or condition of mammalian subject, comprising:

administering a foam produced from a foamable vitamin and or flavonoid composition to a target site, foamable composition comprising:

a. a vitamin and/or flavonoid; and

b. a substantially non-aqueous carrier, the carrier comprising
i. about 50% to about 98% of a polar solvent selected from the group consisting of polyols and polyethylene glycols (PEGs);
ii. 0% to about 48% of a secondary polar solvent;
iii. a surface-active agent or foam adjuvant;
iv. 0% to about 5% by weight of at least one polymeric agent;
v. 0% to about 5% of a conservation agent; and
vi. a propellant at a concentration of about 3% to about 25% by weight of the total composition.

In one or more embodiments there is provided a method of treating a disorder or condition of mammalian subject, comprising:

administering to a target site a foam produced by release of a foamable vitamin and flavonoid composition from a pressurized canister containing a composition to which a vacuum was applied prior to filling with a hydrocarbon propellant, the composition comprising:

a. a vitamin and/or flavonoid; and

b. a substantially non-aqueous carrier, the carrier comprising
i. about 50% to about 98% of a polar solvent selected from the group consisting of polyols and polyethylene glycols (PEGs);
ii. 0% to about 48% of a secondary polar solvent;
iii. a surface-active agent or foam adjuvant;
iv. 0% to about 5% by weight of at least one polymeric agent;
v. 0% to about 5% of a conservation agent; and
vi. a propellant at a concentration of about 3% to about 25% by weight of the total composition,
   wherein applying a vacuum and filling the canister containing the composition with hydrocarbon propellant contributes to stabilize the vitamin against degradation.

In another aspect, a foamable vitamin and or flavonoid carrier is provided including about 50% to about 98% of a polar solvent selected from the group consisting of (1) a polyol and (2) a polyethylene glycol (PEG); 0% to about 48% of a secondary polar solvent; a surface-active agent; about 0.01% to about 5% by weight of at least one polymeric agent; and a liquefied or compressed gas propellant at a concentration of about 3% to about 25% by weight of the total composition.

In one or more embodiments, the compositions further comprise up to 10% of water.

In one or more embodiments, the composition is substantially non-aqueous and/or substantially alcohol-free.

In one or more embodiments, the composition further comprises a therapeutically effective concentration of one or more vitamins. In an additional embodiment, the composition further contains a therapeutically effective concentration of one or more additional therapeutic agents.

In one or more embodiments, the polyol is selected from the group consisting of a diol, a triol and a saccharide, and the triol may be selected from the group consisting of glycerin, butane-1,2,3-triol, butane-1,2,4-triol and hexane-1,2,6-triol, or the diol is selected from the group consisting of propylene glycol, butanediol, butenediol, butynediol, pentanediol, hexanediol, octanediol, neopentyl glycol, 2-methyl-1,3-propanediol, diethylene glycol, triethylene glycol, tetraethylene glycol, dipropylene glycol and dibutylene glycol.

In one or more embodiments, the polyol consists of at least one diol and at least one triol, and wherein the ratio between the diol and triol is between 9:1 and 1:1.

In one or more embodiments, the composition includes a mixture of at least one polyol and at least one PEG, and the PEG may be selected from the group consisting of PEG 200, PEG 300, PEG 400, PEG 600, PEG 1000, PEG 4000, PEG 6000 and PEG 8000, or the composition contains one or more PEGs in a concentration to provide viscosity of less than 12,000 CPs.

In one or more embodiments, the composition includes a secondary polar solvent selected from the group consisting of dimethyl isosorbide, tetrahydrofurfuryl alcohol polyethyleneglycol, ether, DMSO, a pyrrolidone, N-Methyl-2-pyrrolidone, 1-Methyl-2-pyrrolidinone, ethyl proxitol, dimethylacetamide, a PEG-type surfactant, an alpha hydroxy acid, lactic acid and glycolic acid, or the secondary polar solvent is dimethyl isosorbide.

In one or more embodiments, the composition includes (1) at least one polar solvent selected from a diol, a triol and PEG, and (2) at least one secondary polar solvent, and for example, the polar solvent comprises a mixture of at least one polyol and at least one PEG, and for example, the polyol comprises a mixture of at least two polyols.

In one or more embodiments, the ratio between the polyol and/or PEG and the secondary polar solvent is between 9:1 and 1:1.

In yet an additional embodiment, the foamable vitamin composition further contains an additional therapeutic agent.

In another aspect, a method of treating a disorder of mammalian subject includes administering a foamable therapeutic composition to a target area, the composition comprising a therapeutically effective concentration of an active agent, about 50% to about 98% of a polar solvent selected from the group consisting of (1) a polyol; and (2) a polyethylene glycol; 0% to about 48% of a secondary polar solvent; about 0.01% to about 5% by weight of at least one polymeric agent; a surface-active agent; and a liquefied or compressed gas propellant at a concentration of about 3% to about 25% by weight of the total composition.

In one or more embodiments, the target site is selected from the group consisting of the skin, a body cavity, a mucosal surface, the nose, the mouth, the eye, the ear canal, the respiratory system, the vagina and the rectum.

In one or more embodiments, the surface active agent ranges from about less than 0.1% up to about 15% or up to about 20% by weight of composition depending on the surfactant selected or preferably is about 0.2% to about 0.5% by weight of composition.

### DETAILED DESCRIPTION

In one or more embodiments there is provided a hygroscopic vitamin and or flavonoid containing composition comprising:
a. at least one hygroscopic substance at a sufficient concentration to provide an Aw value of the hygroscopic vitamin and or flavonoid containing composition of less than 0.9; and
b. a vitamin and or flavonoid or a derivative thereof or a combinations thereof.

In one or more embodiments there is provided a composition wherein the vitamin is selected from the group consisting of vitamin A, B1, B2, B3, B5, B6, B7, B9, B12, PABA, C, D1, D2, D3, D4, D5, E, K and F and a derivative thereof or combinations thereof.

In one or more embodiments there is provided a composition, wherein the vitamin, flavonoid or a derivative thereof is susceptible to oxidation.

In one or more embodiments there is provided a composition, wherein the vitamin, flavonoid or a derivative thereof is soluble in water.

In one or more embodiments there is provided a composition, wherein the vitamin, flavonoid or a derivative thereof is soluble in a hydrophobic solvent.

In one or more embodiments there is provided a composition wherein the vitamin is a combination of two or more vitamins selected from the group consisting of vitamin A, B3, C, K, E, and F and a derivative thereof.

In one or more embodiments there is provided a composition wherein the vitamin is a combination of two or more vitamins selected from the group consisting of vitamin A, C, D1, D2, D3, D4, and D5 and a derivative thereof.

In one or more embodiments there is provided a composition, wherein the flavonoid is selected from the group consisting of flavonols; flavones flavonones flavan-3-ols anthocyanins and proanthocyanidins.

In one or more embodiments there is provided a composition wherein the flavonoid is selected from the group consisting of benzquercin, diosmin, ethoxazorutoside, flavodate, sodium hesperidin, leucocianido, monoxerutin, oxerutin, quercetin, rutoside, rosmarinic acid, kaempferol, myricetin apigenin, luteolin, hesperetin, naringenin, eriodictyol, cyanidin, delphinidin, malvidin, pelargonidin, peonidin, petunidin, catechin, gallocatechin, epicatechin, and epigallocatechin.

In one or more embodiments there is provided a composition wherein the flanonoid is a synergistic combination of two or more flavonoids selected from the group consisting of benzquercin, diosmin, ethoxazorutoside, flavodate, sodium hesperidin, leucocianido, monoxerutin, oxerutin, quercetin, rutoside, rosmarinic acid, kaempferol, myricetin apigenin, luteolin, hesperetin, naringenin, eriodictyol, cyanidin, delphinidin, malvidin, pelargonidin, peonidin, petunidin, catechin, gallocatechin, epicatechin, and epigallocatechin.

In one or more embodiments there is provided a composition wherein the flanonoid is quercitin and or rosmarinic acid and the vitamin is vitamin A, and or C and or D (D1, D2, D3, D4, or D5) and or E or a derivative thereof.

In one or more embodiments there is provided a composition, wherein the vitamin, flavonoid or a derivative thereof or combinations thereof comprises an antioxidant.

In one or more embodiments there is provided a composition wherein the vitamin, flavonoid or a derivative thereof or combinations thereof improves stimulates or promotes target site metabolism.

In one or more embodiments there is provided a composition wherein the vitamin, flavonoid or a derivative thereof or combinations thereof alleviates, ameliorates, treats, prevents, retards or otherwise has a beneficial effect on a skin or body cavity condition.

In one or more embodiments there is provided a composition wherein the skin condition is selected from the group consisting of skin pigmentation, dry skin, a wound, acne, psoriasis and skin aging.

In one or more embodiments there is provided a composition wherein the vitamin is a combination of two or more vitamins selected from the group comprising vitamin B3, E and C and a derivative thereof.

In one or more embodiments there is provided a composition further comprising a preservative and or modulating agent.

In one or more embodiments there is provided a composition wherein the preservative is selected from the group consisting of disodium metabisulfate and sharomix 824 and the modulating agent is EDTA and or optionally a buffering agent.

In one or more embodiments there is provided a composition, wherein the vitamin and or flavonoid act also as modulating agents.

In one or more embodiments there is provided a composition further comprising an additional therapeutic agent in addition to the vitamin, flavonoid, a derivative thereof or combinations thereof.

In one or more embodiments there is provided a composition, further comprising at least one component, selected from the group consisting of:
a. about 0.01% to about 5% by weight of at least one polymeric agent selected from a bioadhesive agent, a gelling agent, a film forming agent and a phase change agent; and
b. a surface-active agent.

In one or more embodiments there is provided a composition, wherein the hygroscopic substance is selected from the group consisting of:
a. polyethylene glycols (PEGs);
b. surfactants comprising PEG;
c. polyols;
d. monosaccharides, disaccharides, oligosaccharides and sugar alcohols in an amount to provide hygroscopic properties; and
e. honey.

In one or more embodiments there is provided a composition, wherein the Aw value of the composition is selected from the ranges of (1) about 0.8 and about 0.9; (2) about 0.7 and about 0.8; and (3) less than about 0.7.

In one or more embodiments there is provided a composition, further comprising up to 10% of water.

In one or more embodiments there is provided a composition, wherein the composition is substantially non-aqueous and or substantially alcohol-free.

In one or more embodiments there is provided a composition, wherein the vitamin and or flavonoid or derivative thereof or combinations thereof are loaded into microsponges.

In one or more aspects there is provided a hygroscopic vitamin and or flavonoid containing composition comprising:

a. at least one agent selected from the group consisting of vitamins and flavonoids; and

b. at least one hygroscopic solvent at a sufficient concentration to provide an Aw value of the hygroscopic vitamin and or flavonoid containing composition of less than 0.9;

c. a foam stabilizer selected from the group consisting of at least one surface-active agent; at least one polymeric agent selected from the group consisting of a bioadhesive agent, a gelling agent, a film forming agent and a phase change agent and mixtures thereof; and

d. a propellant at a concentration of about 3% to about 25% by weight of the total composition,

wherein the composition is contained in a pressurized container, is substantially flowable and provides a foam upon release.

In one or more aspects, the hygroscopic vitamin and or flavonoid containing composition optionally may further comprise:

i. about 50% to about 98% of a polar solvent

ii. 0% to about 48% of a secondary polar solvent;

iii. a foam adjuvant;

iv. 0% to about 5% by weight of at least one polymeric agent; and

v. 0% to about 5% of a conservation agent.

In one or more embodiments there is provided a foamable composition comprising a stabilized-vitamin, comprising:

a water-unstable vitamin;

an optional flavonoid; and

a foamable carrier comprising:
i. water;
ii. a foam stabilizer selected from the group consisting of at least one surface-active agent; at least one polymeric agent selected from the group consisting of a bioadhesive agent, a gelling agent, a film forming agent and a phase change agent and mixtures thereof;
iii. about 0.005% to about 5% conservational agent; and
iv. a propellant at a concentration of about 3% to about 25% by weight of the total composition,

where said foamable carrier reduces the destabilizing effect of water on the vitamin, and

wherein the composition is contained in a pressurized container, is substantially flowable and provides a foam upon release.

In one or more embodiments the vitamin, flavonoid, stabilizer, conservational agent, and solvent to the extent they are present are selected to provide a composition that is substantially resistant to aging.

In one or more embodiments the flavonoid and or conservational agent can substantially conserve or stabilize the vitamin.

In one or more embodiments the formulation comprises water, and the flavonoid and or conservational agent can substantially conserve or stabilize the vitamin and further the vitamin, flavonoid, stabilizer, conservational agent, and solvent provide a composition that is substantially resistant to aging.

In one or more embodiments there is provided a foamable composition wherein the Aw value of the composition is selected from the ranges of (1) about 0.8 and about 0.9; (2) about 0.7 and about 0.8; and (3) less than about 0.7.

In one or more embodiments there is provided a foamable composition wherein the surface active agent is about 0.2% to about 5% by weight of composition.

In one or more embodiments there is provided a foamable composition wherein the polar solvent is a polyol is selected from the group consisting of a diol, a triol and a saccharide.

In one or more embodiments there is provided a foamable composition wherein the diol is selected from the group consisting of propylene glycol, butanediol, butenediol, butynediol, pentanediol, hexanediol, octanediol, neopentyl glycol, 2-methyl-1,3-propanediol, diethylene glycol, triethylene glycol, tetraethylene glycol, dipropylene glycol and dibutylene glycol and, wherein the triol is selected from the group consisting of glycerin, butane-1,2,3-triol, butane-1,2,4-triol and hexane-1,2,6-triol.

In one or more embodiments there is provided a foamable composition wherein the polar solvent is a PEG is selected from the group consisting of PEG 200, PEG 300, PEG 400, PEG 600, PEG 1000, PEG 4000, PEG 6000 and PEG 8000.

In one or more embodiments there is provided a foamable composition wherein the carrier composition comprises a polar solvent mixture of at least one polyol and at least one PEG.

In one or more embodiments there is provided a foamable composition, which is non aqueous or substantially non aqueous wherein the secondary polar solvent is selected from the group consisting of dimethyl isosorbide, tetrahydrofurfuryl alcohol polyethyleneglycol, ether, DMSO, a pyrrolidone, N-Methyl-2-pyrrolidone, 1-Methyl-2-pyrrolidinone, ethyl proxitol, dimethylacetamide, a PEG-type surfactant, an alpha hydroxy acid, lactic acid and glycolic acid.

In one or more embodiments there is provided a foamable composition, which is non aqueous or substantially non aqueous wherein the carrier composition comprises (1) at least one polar solvent selected from a diol, a triol and PEG, and (2) at least one secondary polar solvent.

In one or more embodiments there is provided a foamable composition, which is non aqueous or substantially non aqueous wherein the concentration of the polar solvent and the secondary polar solvent is sufficient to provide an Aw value of the hygroscopic pharmaceutical composition of less than 0.9.

In one or more embodiments there is provided a foamable composition, which is non aqueous or substantially non aqueous wherein the Aw value of the composition is selected from the ranges of (1) about 0.8 and about 0.9; (2) about 0.7 and about 0.8; and (3) less than about 0.7.

In one or more embodiments there is provided a foamable composition, wherein the polymeric agent is selected from the group consisting of locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, an amine-bearing polymer, chitosan, alginic acid, hyaluronic acid, a chemically modified starch, a carboxyvinyl polymer, polyvinylpyrrolidone, polyvinyl alcohol, a polyacrylic acid polymer, a polymethacrylic acid polymer, polyvinyl acetate, a polyvinyl chloride polymer, a polyvinylidene chloride polymer, methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, a cationic cellulose PEG 1000, PEG 4000, PEG 6000 and PEG 8000 of Carbopol® 934, Carbopol® 940, Carbopol® 941, Carbopol® 980, Carbopol® 981. hydroxypropylcellulose and carbomer.

In one or more embodiments there is provided a foamable composition, wherein the polymeric agent is dispersible in the polyol or in the mixture of a polyol and an additional polar solvent.

In one or more embodiments there is provided a foamable composition, wherein the surface active agent is selected from the group consisting of a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59, a polyoxyethylene alkylyl ether, polyoxyethylene cetyl ether, polyoxyethylene palmityl ether, polyethylene oxide hexadecyl ether, polyethylene glycol cetyl ether, brij 38, brij 52, brij 56 brij 72, brij 721 and brij W1, a sucrose ester, a partial ester of sorbitol, sorbitan monolaurate, sorbitan monolaurate a monoglyceride, a diglyceride, isoceteth-20, a sucrose ester, or selected from the group consisting of steareth 2, glyceryl monostearate/PEG 100 stearate, Glyceryl Stearate, Steareth-21, peg 40 stearate, polysorbate 60, polysorbate 80, sorbitan stearate, laureth 4, Sorbitan monooleate, ceteareth 16 ceteareth 20, steareth 10, steareth 20, ceteth 20, Macrogol Cetostearyl Ether, ceteth 2, PEG-30 Dipolyhydroxystearate, sucrose distearate, polyoxyethylene (100) stearate, PEG 40 stearate, PEG 100 stearate, laureth 4, cetomacrogol ether, Cetearyl alcohol, Cetearyl glucoside, Oleyl alcohol, Steareth-2, Diisopropyl adipate, Capric/caprilic triglicerides, Polysorbate 20; Polysorbate 80, Montanov 68 (cetearyl alcohol (and) cetearyl glucoside.), Simusol 165 (Glyceryl stearate and PEG-100 stearate). Methyl glucose sequistearate, Peg 30 dipolyhydroxystearate, sucrose stearic acid esters, sorbitan laureth, sorbitan stearate, polyglyceryl-10 laurate, epikuuron 80, span 80 and mixtures thereof.

In one or more embodiments there is provided a foamable composition, further comprises a hydrophobic solvent.

In one or more embodiments there is provided a foamable composition, wherein the hydrophobic solvent is selected from the group consisting of mineral oil, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, unsaturated or polyunsaturated oils, such as olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils; essential oils; and silicone oils, such as dimethicone, cyclomethicone, polyalkyl siloxane, polyaryl siloxane, polyalkylaryl siloxane, a polyether siloxane copolymer and a poly(dimethylsiloxane)-(diphenyl-siloxane) copolymer.

In one or more embodiments there is provided a foamable composition, further comprising a foam adjuvant selected from the group consisting of a fatty alcohol, a fatty acid and a hydroxyl fatty acid or liquid wax.

In one or more embodiments there is provided a foamable composition, further comprising an additional component selected from the group consisting of an anti perspirant, an anti-static agent, a buffering agent, a bulking agent, a chelating agent, a colorant, a conditioner, a deodorant, a diluent, a dye, an emollient, fragrance, a humectant, an occlusive agent, a penetration enhancer, a perfuming agent, a permeation enhancer, a pH-adjusting agent, a preservative, a skin penetration enhancer, a sunscreen, a sun blocking agent, and a sunless tanning agent.

In one or more embodiments there is provided a foamable composition, further comprising at least one additional therapeutic agent in addition to the vitamin, and or flavanoid, a derivative thereof or combinations thereof in a therapeutically effective concentration.

In one or more embodiments there is provided a foamable composition, wherein the vitamin and or flavonoid or derivative thereof or combinations thereof are loaded into microsponges.

In one or more embodiments there is provided a method of treating a disorder or condition of mammalian subject, comprising:

administering a foam produced by release of a foamable vitamin and flavonoid composition from a pressurized canister to a target site, the composition comprising:

a. a vitamin and/or flavonoid; and

b. a substantially non-aqueous carrier, the carrier comprising
i. about 50% to about 98% of a polar solvent selected from the group consisting of polyols and polyethylene glycols (PEGs);
ii. 0% to about 48% of a secondary polar solvent;
iii. a surface-active agent or foam adjuvant;
iv. 0% to about 5% by weight of at least one polymeric agent;
v. 0% to about 5% of a conservation agent; and
vi. a propellant at a concentration of about 3% to about 25% by weight of the total composition.

In other embodiments, a method of treating a disorder or condition of mammalian subject, includes:

administering a foam produced from a foamable vitamin and or flavonoid composition to a target site, the composition comprising:
a foamable composition comprising a stabilized-vitamin, comprising:
   a therapeutically effective amount of a water-unstable vitamin;
   an optional flavonoid; and
   a foamable carrier comprising:
      i. water;
      ii. a foam stabilizer selected from the group consisting of at least one surface-active agent; at least one polymeric agent selected from the group consisting of a bioadhesive agent, a gelling agent, a film forming agent and a phase change agent and mixtures thereof;
      iii. about 0.005% to about 5% conservational agent; and
      iv. a propellant at a concentration of about 3% to about 25% by weight of the total composition,

where said foamable carrier reduces the destabilizing effect of water on the vitamin, and

wherein the composition is contained in a pressurized container, is substantially flowable and provides a foam upon release.

In one or more embodiments there is provided a method, wherein the target site is selected from the group consisting of the skin, a body cavity, a mucosal surface, the nose, the mouth, the eye, the ear canal, the respiratory system, the vagina and the rectum.

In one or more embodiments there is provided a method, wherein the disorder is selected from the group consisting of dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, ecthyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum; and wherein the active agent is suitable for treating said disorder.

In one or more embodiments there is provided a method, wherein the at least one additional therapeutic agent is selected from the group consisting of active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, wound healing agents and wart removers.

In one or more embodiments there is provided, wherein the at least one active agent is selected from the group consisting of alclometasone dipropionate, amcinafel, amcinafide, amcinonide, beclomethasone, beclomethasone dipropionate, betamethsone, betamethasone benzoate, betamethasone dexamethasone-phosphate, dipropionate, betamethasone valerate, budesonide, chloroprednisone, chlorprednisone acetate, clescinolone, clobetasol, clobetasol propionate, clobetasol valerate, clobetasone, clobetasone butyrate, clocortelone, cortisone, cortodoxone, craposone butyrate, desonide, desoxymethasone, dexamethasone, desoxycorticosterone acetate, dichlorisone, diflorasone diacetate, diflucortolone valerate, difluorosone diacetate, diflurprednate, fluadrenolone, flucetonide, flucloronide, fluclorolone acetonide, flucortine butylesters, fludroxycortide, fludrocortisone, flumethasone, flumethasone pivalate, flumethasone pivalate, flunisolide, fluocinolone, fluocinolone acetonide, fluocinonide, fluocortin butyl, fluocortolone, fluorometholone, fluosinolone acetonide, fluperolone, fluprednidene acetate, fluprednisolone hydrocortamate, fluradrenolone, fluradrenolone acetonide, flurandrenolone, fluticasone, halcinonide, halobetasol, hydrocortisone, hydrocortisone acetate, hydrocortisone butyrate, hydrocortisone cyclopentylpropionate, hydrocortisone valerate, hydroxyltriamcinolone, medrysone, meprednisone, .alpha.-methyl dexamethasone, methylprednisolone, methylprednisolone acetate, mometasone furoate, paramethasone, prednisolone, prednisone, pregnenolone, progesterone, spironolactone, triamcinolone, triamcinolone acetonide and derivatives, esters and salts thereof.

In one or more embodiments there is provided a method, wherein the at least one additional therapeutic agent is an anti-infective agent.

In one or more embodiments there is provided a method, wherein the at least one anti-infective agent is selected from the group consisting of an antibiotic agent, an antibacterial agent, an antifungal agent, an agent that controls yeast, an antiviral agent and an antiparasitic agent.

In one or more embodiments there is provided a method, wherein the antifungal agent is selected from the group consisting of a polyene, natamycin, nystatin; an allylamine, naftifine, terbinafine; an imidazole, bifonazole, clotrimazole, econazole, fenticonazole, ketocanazole, miconazole, oxiconazole; a diazole, a triazoles, fluconazole, itraconazole, terconazole, tolnaftate, ciclopirox, undecylenic acid, sulbentine, griseofulvin, Amphotericin B, flucytosine (5FC), a morpholine compound, amorolfine, and the related morpholines and analogs, derivatives and salts thereof, and any combination thereof at a therapeutically effective concentration.

In one or more embodiments there is provided a method, wherein the antibacterial agent is selected from the group consisting of a beta-lactam antibiotic, an aminoglycoside, an ansa-type antibiotic, an anthraquinone, an azole, metronidazole, an antibiotic glycopeptide, a macrolide, erythromycin, clindamycin, an antibiotic nucleoside, an antibiotic peptide, polymyxin B, an antibiotic polyene, an antibiotic polyether, an antibiotic quinolone, an antibiotic steroid, fucidic acid, mupirocin, chloramphenicol, a sulfonamide, tetracycline, an antibiotic metal, silver, copper, zinc, mercury, tin, lead, bismuth, cadmium, chromium, an oxidizing agent, iodine, iodate, a periodate, a hypochlorite, a permanganate, a substance that release free radicals and/or active oxygen, a cationic antimicrobial agent, a quaternary ammonium compound, a biguanide, chlorohexidine, a triguanide, a bisbiguanide, a polymeric biguanide, a naturally occurring antibiotic compound and analogs, derivatives, salts, ions and complexes thereof.

In one or more embodiments there is provided a method, wherein the vitamin or at least one additional therapeutic agent is unstable in the presence of water.

In one or more embodiments there is provided a method, comprising (1) at least one vitamin; and (2) at least one additional therapeutic agent selected from the group of an antifungal agent, an antimicrobial agent, an antiviral agent, an anti-acne agent, an antipsoriasis agent, a keratolytic agent and an antiacne agent.

In one or more embodiments there is provided a method, wherein the additional therapeutic agent comprises a combination of (1) a corticosteroid; and (2) and active agent selected from the group of an anti-infective agent, an antifungal agent, an antimicrobial agent, an antiviral agent, an anti-acne agent, a retinoid, a vitamin D, a vitamin D3 derivative, calcipotriol, calcitriol and antipsoriasis agent, a keratolytic agent, an anti-proliferative agent, an anti-cancer agent, a non-steroidal anti-inflammatory agent, an immunomodulator, an immunosuppressant and an anti-rosacea agent.

In one or more embodiments there is provided a method, wherein the additional therapeutic agent comprises a combination of (1) a keratolytic agent; and (2) and active agent selected from the group of a corticosteroid, an anti-infective agent, an antifungal agent, an antimicrobial agent, an antiviral agent, an anti-acne agent, a retinoid, a vitamin D, a vitamin D3 derivative, calcipotriol, calcitriol and antipsoriasis agent, an anti-proliferative agent, an anti-cancer agent, a non-steroidal anti-inflammatory agent, an immunomodulator, an immunosuppressant and an anti-rosacea agent.

According to one or more embodiments, the foamable vitamin and flavonoid carrier, comprises:
a. about 50% to about 98% of a polar solvent selected from the group consisting of (1) a polyol and (2) a polyethylene glycol (PEG);
b. 0% to about 48% of a secondary polar solvent;
c. a surface-active agent;
d. about 0.01% to about 5% by weight of at least one polymeric agent; and
e. a liquefied or compressed gas propellant at a concentration of about 3% to about 25% by weight of the total composition.

All % values are provided on a weight (w/w) basis.

In one or more embodiments, the surface active agent ranges from about less than 0.1% upto about 15% or upto about 20% by weight of composition depending on the surfactant selected or preferably is about 0.2% to about 0.5% by weight of composition.

Water, up to 25% of the composition, and more preferably up to 10%, and optional ingredients are added to complete the total mass to 100%. In certain cases, the composition contains two active agents that require different pH environments in order to remain stable. For example, corticosteroids are typically stable at acidic pH (they have a maximum stability at a pH of about 4-6) and vitamin D analogues are typically stable at basic pH (they have a maximum stability at pH values above about 8). In other cases, the active agent degrades in the presence of water, and therefore, in such cases the present of water in the composition is not desirable. Thus, in certain preferred embodiments, the composition is substantially non-aqueous.

Upon release from an aerosol container, the foamable carrier forms an expanded foam suitable for the treatment of an infected surface and for topical administration to the skin, a body surface, a body cavity or a mucosal surface.

Polar Solvent

A “polar solvent” is an organic solvent, typically soluble in both water and oil. Certain polar solvents, for example propylene glycol and glycerin, possess the beneficial property of a humectants.

The identification of a “polar solvent”, as used herein, is not intended to characterize the solubilization capabilities of the solvent for any specific active agent or any other component of the foamable composition. Rather, such information is provided to aid in the identification of materials suitable for use as a part in the foamable compositions described herein.

Polyol

In an embodiment, the polar solvent is a polyol. A polyol is an organic substance that contains at least two hydroxy groups in its molecular structure.

In one or more embodiments, the foamable carrier contains at least one diol (a compound that contains two hydroxy groups in its molecular structure). Examples of diols include propylene glycol (e.g., 1,2-propylene glycol and 1,3-propylene glycol), butanediol (e.g., 1,2-butanediol, 1,3-butanediol, 2,3-butanediol and 1,4-butanediol), butanediol (e.g., 1,3-butanediol and 1,4-butenediol), butynediol, pentanediol (e.g., pentane-1,2-diol, pentane-1,3-diol, pentane-1,4-diol, pentane-1,5-diol, pentane-2,3-diol and pentane-2,4-diol), hexanediol (e.g., hexane-1,6-diol hexane-2,3-diol and hexane-2,56-diol), octanediol (e.g., 1,8-octanediol), neopentyl glycol, 2-methyl-1,3-propanediol, diethylene glycol, triethylene glycol, tetraethylene glycol, dipropylene glycol and dibutylene glycol.

In one or more embodiments, the foamable carrier contains at least one triol (a compound that contains three hydroxy groups in its molecular structure), such as glycerin, butane-1,2,3-triol, butane-1,2,4-triol and hexane-1,2,6-triol.

In one or more embodiments, the polyol is a mixture of polyols. In one or more embodiments, the mixture of polyols contains at least one diol and at least one triol. According to certain embodiments the ratio between the diol and triol is between 9:1 and 1:1.

In one or more embodiments, part of mixture of polyols is a saccharide. Exemplary saccharides include, but are not limited to monosaccharide, disaccharides, oligosaccharides and sugar alcohols.

A monosaccharide is a simple sugar that cannot be hydrolyzed to smaller units. Empirical formula is (CH2O)n and range in size from trioses (n=3) to heptoses (n=7). Exemplary monosaccharide compounds are ribose, glucose, fructose and galactose.

Disaccharides are made up of two monosaccharides joined together, such as sucrose, maltose and lactose.

A sugar alcohol (also known as a polyol, polyhydric alcohol, or polyalcohol) is a hydrogenated form of saccharide, whose carbonyl group (aldehyde or ketone, reducing sugar) has been reduced to a primary or secondary hydroxyl group. They are commonly used for replacing sucrose in foodstuffs, often in combination with high intensity artificial sweeteners to counter the low sweetness. Some exemplary sugar alcohols, which are suitable for use are mannitol, sorbitol, xylitol, maltitol, lactitol. (Maltitol and lactitol are not completely hydrogenated compounds—they are a monosaccharide combined with a polyhydric alcohol). Mixtures of polyols, including (1) at least one polyol selected from a diol and a triol; and (2) a saccharide are contemplated.

Polyethylene Glycol

In an embodiment, the polar solvent consists of a polymerized ethylene glycol, namely polyethylene glycol, which is also termed “PEG”. Exemplary PEGs are provided in the following table.

| | Av. Molecular | | |
|---|---|---|---|
| Composition | weight | Appearance | Melting point (° C.) |
| PEG 200 | 190˜210 | Oily liquid | |
| PEG 300 | 285˜315 | Oily liquid | |
| PEG 400 | 380˜420 | Oily liquid | |
| PEG 600 | 570˜630 | Oily liquid | 17˜22 |
| PEG 1000 | 950˜1050 | Solid | 35˜40 |
| PEG 4000 | 3800˜4400 | Solid | 53˜58 |
| PEG 6000 | 5600˜6400 | Solid | 55˜60 |
| PEG 8000 | 7500˜8500 | Solid | 58˜65 |

Thus, in an embodiment, the PEG is selected from the group consisting of PEG 200, PEG 300, PEG 400, PEG 600, PEG 1000, PEG 4000, PEG 6000, PEG 8000 and PEG 10,000. The foamable carrier can contain a single PEG or a mixture of two or more PEGs. PEGs having molecular weight of more that about 1000 possess gelling properties; i.e., they increase the viscosity of a composition. Therefore, by combining PEGs with different molecular weights/melting points, one can attain varying levels of flowability as desirable for the treatment of a given target site. Small amounts of high molecular weight PEG added to low molecular weight PEG can substantially add to the viscosity. Thus, the amount of high molecular weight PEG that may usefully be added to foamable compositions is such that the composition remains flowable and does not form a block. The concentration of the PEG should ideally be in a level that results in viscosity, prior to filling of the composition into aerosol canisters, of less than 12,000 CPs, and more preferably, less than 10,000 CPs.

Secondary Polar Solvent

Optionally, a secondary polar solvent is added to the foamable composition. The secondary polar solvent is selected from a variety of organic solvents that are typically miscible on both water and oil. Examples of polar solvent that can be contained in the foamable carrier include dimethyl isosorbide, tetrahydrofurfuryl alcohol polyethyleneglycol ether (glycofurol), DMSO, pyrrolidones, (such as N-Methyl-2-pyrrolidone and 1-Methyl-2-pyrrolidinone), ethyl proxitol, dimethylacetamide (DMAc), PEG-type surfactants and alpha hydroxy acids, such as lactic acid and glycolic acid.

Solubilization and Penetration Enhancement

In many cases, polyols, PEGs and polar solvents possess a high solubilizing power and thus, they can enable increased concentrations of an active agent. Polyols, PEGs and polar solvents are also known for their skin penetration enhancement properties. These properties enable high drug bioavailability in the target area of treatment, resulting in an enhanced therapeutic effect. Occasionally, combinations of a polyol, PEGs and a secondary polar solvent, exhibit an increased permeability across the skin, as suggested, for example, in Eur J Pharm Biopharm. 1998 November; 46(3):265-71.

Thus, in one or more embodiments, the foamable carrier contains (1) at least one polar solvent, selected from a polyol (selected from a diol and a triol) and PEG; and (2) at least one secondary polar solvent.

In one or more embodiments, the foamable carrier contains (1) a mixture of at least two polyols; and (2) at least one secondary polar solvent. In additional embodiments, the foamable carrier contains a mixture of at least one polyol and at least one PEG; yet in other embodiments the foamable carrier contains (1) a mixture of at least one polyol and at least one PEG and (2) at least one secondary polar solvent.

According to certain embodiments the ratio between the polyol and/or PEG and the secondary polar solvent is between 9:1 and 1:1.

In certain embodiments, the polyol is selected from the group consisting of propylene glycol, hexylene glycol and glycerin (and mixtures thereof); and the secondary polar solvent is selected from the group consisting of dimethyl isosorbide, diethylene glycol monoethyl ether, a liquid polyethylene glycol and glycofurol.

In certain embodiments, the foamable carrier contains (1) at least one polyol; and (2) dimethyl isosorbide.

Other Non limiting examples include dimethyl isosorbide polyols, diethylene glycol, propylene glycol n-alkanols, terpenes, di-terpenes, tri-terpenes, limonene, terpene-ol, 1-menthol, dioxolane, ethylene glycol, other glycols, oleyl alcohol, alpha-hydroxy acids, such as lactic acid and glycolic acid, sulfoxides, such as dimethylsulfoxide (DMSO), dimethylformanide, methyl dodecyl sulfoxide, dimethylacetamide, azone (1-dodecylazacycloheptan-2-one), 2-(n-nonyl)-1,3-dioxolane, alkanols, such as dialkylamino acetates, and admixtures thereof. In certain preferred embodiments, the polar solvent is selected from the group consisting of dimethyl isosorbide glycerol (glycerin), propylene glycol, hexylene glycol, terpene-ol, oleyl alcohol, lactic acid and glycolic acid.

Short chain alcohols, such as ethanol and propanol are known as polar solvents, however, according to one or more embodiments, the composition is substantially alcohol-free, i.e., free of short chain alcohols. Short chain alcohols, having up to 5 carbon atoms in their carbon chain skeleton and one hydroxyl group, such as ethanol, propanol, isopropanol, butanol, iso-butanol, t-butanol and pentanol, are considered less desirable polar solvents due to their skin-irritating effect.

Thus, in certain embodiments, the composition is substantially alcohol-free and includes less than about 5% final concentration of lower alcohols, preferably less than about 2%, more preferably less than about 1%. However, in other embodiments, a short chain alcohol can be included in the composition, as long as the ratio between the short chain alcohol and the polyol is less than 1:4 by weight.

Polymeric Agent

The composition contains a polymeric agent. The presence of a polymeric agent can be necessary for or enhance the creation of foam, having fine bubble structure, which does not readily collapse upon release from a pressurized aerosol can. The polymeric agent can serve to stabilize the foam composition and to control drug residence in the target organ. Although it has been surprisingly observed that in compositions with very high oil or wax or fatty acid or fatty alcohol content the presence of polymeric agent can decrease foam quality. Preferably, the polymeric agent is soluble or readily dispersible in the polyol; or in the mixture of a polyol and an additional polar solvent.

Non-limiting examples of polymeric agents that are soluble or readily dispersible in propylene glycol are Hydroxypropylcellulose and carbomer (homopolymer of acrylic acid is crosslinked with an allyl ether pentaerythritol, an allyl ether of sucrose, or an allyl ether of propylene, such as Carbopol® 934, Carbopol® 940, Carbopol® 941, Carbopol® 980 and Carbopol® 981.

Other polymeric agents are suitable for use provided that they are soluble or readily dispersible in the polyol; or in the mixture of a polyol and an additional polar solvent, on a case by case basis.

Exemplary polymeric agents include, in a non-limiting manner, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, amine-bearing polymers such as chitosan; acidic polymers obtainable from natural sources, such as alginic acid and hyaluronic acid; chemically modified starches and the like, carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like.

Additional exemplary polymeric agents include semi-synthetic polymeric materials such as cellulose ethers, such as methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, and cationic celluloses pemulen and aluminum starch octenylsuccinate (ASOS). Polyethylene glycol, having molecular weight of 1000 or more (e.g., PEG 1,000, PEG 4,000, PEG 6,000 and PEG 10,000) also have gelling capacity and while they are considered herein as “secondary polar solvents”, as detailed herein, they are also considered polymeric agents.

In one or more embodiments the polymeric agents have emulsifying properties. In certain preferred embodiments the polymeric agent is a derivatized hydrophilic polymer with hydrophobic alkyl moieties Other types that may also a similar stabilizing effect are silicone copolymers and derivatized starch ASOS.

Mixtures of the above polymeric agents are contemplated.

The concentration of the polymeric agent should be selected so that the composition, after filling into aerosol canisters, is flowable, and preferably can be shaken in the canister. In one or more embodiments, the concentration of the polymeric agent is selected such that the viscosity of the composition, prior to filling of the composition into aerosol canisters, is less than 12,000 CPs, and more preferably, less than 10,000 CPs.

Surface Active Agent

The composition further contains a surface-active agent. Surface-active agents (also termed “surfactants”) include any agent linking oil and water in the composition, in the form of emulsion. A surfactant's hydrophilic/lipophilic balance (HLB) describes the emulsifier's affinity toward water or oil. HLB is defined for non-ionic surfactants. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average). In many cases a single surfactant may suffice. In other cases a combination of two or more surfactants is desired. Reference to a surfactant in the specification can also apply to a combination of surfactants or a surfactant system. As will be appreciated by a person skilled in the art which surfactant or surfactant system is more appropriate is related to the vehicle and intended purpose. In general terms a combination of surfactants is usually preferable where the vehicle is an emulsion. In an emulsion environment a combination of surfactants can be significant in producing breakable forms of good quality. It has been further discovered that the generally thought considerations for HLB values for selecting a surfactant or surfactant combination are not always binding for emulsions and that good quality foams can be produced with a surfactant or surfactant combination both where the HLB values are in or towards the lipophilic side of the scale and where the HLB values are in or towards the hydrophilic side of the scale. Surfactants also play a role in foam formation where the foamable formulation is a single phase composition.

According to one or more embodiments the composition contains a single surface active agent having an HLB value between about 2 and 9, or more than one surface active agent and the weighted average of their HLB values is between about 2 and about 9. Lower HLB values may in certain embodiments be more applicable to water in oil emulsions.

According to one or more embodiments the composition contains a single surface active agent having an HLB value between about 7 and 14, or more than one surface active agent and the weighted average of their HLB values is between about 7 and about 14. Mid range HLB values may in certain embodiments be more suitable for oil in water emulsions.

According to one or more other embodiments the composition contains a single surface active agent having an HLB value between about 9 and about 19, or more than one surface active agent and the weighted average of their HLB values is between about 9 and about 19. In a waterless or substantially waterless environment a wide range of HLB values may be suitable.

Preferably, the composition contains a non-ionic surfactant. Nonlimiting examples of possible non-ionic surfactants include a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59; a polyoxyethylene alkyl ether, polyoxyethylene cetyl ether, polyoxyethylene palmityl ether, polyethylene oxide hexadecyl ether, polyethylene glycol cetyl ether, steareths such as steareth 2, brij 21, brij 721, brij 38, brij 52, brij 56 and brij W1, a sucrose ester, a partial ester of sorbitol and its anhydrides, sorbitan monolaurate, sorbitan monolaurate, a monoglyceride, a diglyceride, isoceteth-20 and mono-, di- and tri-esters of sucrose with fatty acids. In certain embodiments, suitable sucrose esters include those having high monoester content, which have higher HLB values.

In certain embodiments, surfactants are selected which can provide a close packed surfactant layer separating the oil and water phases. To achieve such objectives combinations of at least two surfactants are selected. Preferably, they should be complex emulgators and more preferably they should both be of a similar molecular type. For example, a pair of ethers like steareth 2 and steareth 21, or a pair of esters for example, PEG-40 stearate and polysorbate 80. In certain circumstances POE esters cannot be used and a combination of sorbitan laurate and sorbitan stearate or a combination of sucrose stearic acid ester mixtures and sodium laurate may be used. All these combinations due to their versatility and strength may also be used satisfactorily and effectively with solutions and with solid/crystalline suspensions, although the amounts and proportion may be varied according to the formulation and its objectives as will be appreciated by a man of the art.

It has been discovered also that by using a derivatized hydrophilic polymer with hydrophobic alkyl moieties as a polymeric emulsifier such as pemulen it is possible to stabilize the emulsion better about or at the region of phase reversal tension. Other types of derivatized polymers like silicone copolymers, derivatized starch [Aluminum Starch Octenylsuccinate (ASOS)]/[DRY-FLO AF Starch], and derivatized dexrin may also a similar stabilizing effect.

A series of dextrin derivative surfactants prepared by the reaction of the propylene glycol polyglucosides with a hydrophobic oxirane-containing material of the glycidyl ether are highly biodegradable. [Hong-Rong Wang and Keng-Ming Chen, Colloids and Surfaces A: Physicochemical and Engineering Aspects Volume 281, Issues 1-3, 15 Jun. 2006, Pages 190-193].

Non-limiting examples of non-ionic surfactants that have HLB of about 7 to about 12 include steareth 2 (HLB˜4.9); glyceryl monostearate/PEG 100 stearate (Av HLB˜11.2); stearate Laureth 4 (HLB˜9.7) and cetomacrogol ether (e.g., polyethylene glycol 1000 monocetyl ether).

Non-limiting examples of preferred surfactants, which have a HLB of 4-19 are set out in the Table below:

| Surfactant | HLB |
|---|---|
| steareth 2 | ˜4.9 |
| glyceryl monostearate/PEG 100 stearate | Av ˜11.2 |
| Glyceryl Stearate | ˜4 |
| Steareth-21 | ˜15.5 |
| peg 40 stearate | ˜16.9 |
| polysorbate 80 | ˜15 |
| sorbitan stearate | ˜4.7 |
| laureth 4 | ˜9.7 |
| Sorbitan monooleate (span 80) | ˜4.3 |
| ceteareth 20 | ˜15.7 |
| steareth 20 | ˜15.3 |
| ceteth 20 | ˜15.7 |
| Macrogol Cetostearyl Ether | ˜15.7 |
| ceteth 2 (Lipocol C-2) | ˜5.3 |
| PEG-30 Dipolyhydroxystearate | ˜5.5 |
| sucrose distearate (Sisterna SP30) | ˜6 |
| polyoxyethylene (100) stearate | ˜18.8 |

More exemplary stabilizing surfactants which may be suitable for use are found below.

PEG-Fatty Acid Monoester Surfactants

| Chemical name | Product example name | HLB |
|---|---|---|
| PEG-30 stearate | Myrj 51 | >10 |
| PEG-40 laurate | Crodet L40 (Croda) | 17.9 |
| PEG-40 oleate | Crodet O40 (Croda) | 17.4 |
| PEG-45 stearate | Nikkol MYS-45 (Nikko) | 18 |
| PEG-50 stearate | Myrj 53 | >10 |
| PEG-100 stearate | Myrj 59, Arlacel 165 (ICI) | 19 |

PEG-Fatty Acid Diester Surfactants:

| Chemical name | Product example name | HLB |
|---|---|---|
| PEG-4 dilaurate | Mapeg .RTM. 200 DL (PPG), | 7 |
| | Kessco .RTM.PEG 200 DL | |
| | (Stepan), LIPOPEG 2-DL (Lipo | |
| | Chem.) | |
| PEG-4 | distearate Kessco .RTM. 200 | 5 |
| | DS (Stepan.sub) | |
| PEG-32 dioleate | Kessco .RTM. PEG 1540 DO | 15 |
| | (Stepan) | |
| PEG-400 dioleate | Cithrol 4DO series (Croda) | >10 |
| PEG-400 distearate | Cithrol 4DS series (Croda) | >10 |
| PEG-20 glyceryl oleate | Tagat .RTM. O (Goldschmidt) | >10 |

Transesterification Products of Oils and Alcohols

| Chemical name | Product example name | HLB |
|---|---|---|
| PEG-30 castor oil | Emalex C-30 (Nihon Emulsion) | 11 |
| PEG-40 hydrogenated castor oil | Cremophor RH 40 (BASF), | 13 |
| | Croduret (Croda), Emulgin | |
| | HRE 40 (Henkel) | |

Polyglycerized Fatty Acids, such as:

| Chemical name | Product example name | LB |
|---|---|---|
| Polyglyceryl-6 dioleate | Caprol .RTM. 6G20 (ABITEC); | 8.5 |
| | PGO-62 (Calgene), PLUROL | |
| | OLEIQUE CC 497 | |
| | (Gattefosse)Hodag | |

PEG-Sorbitan Fatty Acid Esters

| Chemical name | Product example name | HLB |
|---|---|---|
| PEG-20 sorbitan | Tween-20 (Atlas/ICI), Crillet 1 | 17 |
| monolaurate | (Croda), DACOL MLS 20 | |
| | (Condea) | |
| PEG-20 sorbitan | Tween 40 (Atlas/ICI), Crillet 2 | 16 |
| Monopalmitate | (Croda) | |
| PEG-20 sorbitan | Tween-60 (Atlas/ICI), Crillet 3 | 15 |
| monostearate | (Croda) | |
| PEG-20 sorbitan | Tween-80 (Atlas/ICI), Crillet 4 | 15 |
| monooleate | (Croda) | |

Polyethylene Glycol Alkyl Ethers

| Chemical name | Product example name | HLB |
|---|---|---|
| PEG-2 oleyl ether | oleth-2 Brij 92/93 (Atlas/ICI) | 4.9 |
| PEG-3 oleyl ether | oleth-3 Volpo 3 (Croda) | <10 |
| PEG-5 oleyl ether | oleth-5 Volpo 5 (Croda) | <10 |
| PEG-10 oleyl ether | oleth-10 Volpo 10 (Croda), Brij | 12 |
| | 96/97 (Atlas/ICI) | |
| PEG-20 oleyl ether | oleth-20 Volpo 20 (Croda), Brij | 15 |
| | 98/99 (Atlas/ICI) | |
| PEG-4 lauryl ether | laureth-4Brij 30 (Atlas/ICI) | 9.7 |
| PEG-23 lauryl ether | laureth-23Brij 35 (Atlas/ICI) | 17 |
| PEG-10 stearyl ether | Brij 76 (ICI) | 12 |
| PEG-2 cetyl ether | Brij 52 (ICI) | 5.3 |

Sugar Ester Surfactants

| Chemical name | Product example name | HLB |
|---|---|---|
| Sucrose distearate | Sisterna SP50, Surfope 1811 | 11 |

Sorbitan Fatty Acid Ester Surfactants

| Chemical name | Product example name | HLB |
|---|---|---|
| Sorbitan monolaurate | Span-20 (Atlas/ICI), Crill 1 | 8.6 |
| | (Croda), Arlacel 20 (ICI) | |
| Sorbitan monopalmitate | Span-40 (Atlas/ICI), Crill 2 | 6.7 |
| | (Croda), Nikkol SP-10 (Nikko) | |
| Sorbitan monooleate | Span-80 (Atlas/ICI), Crill 4 | 4.3 |
| | (Croda), Crill 50 (Croda) | |
| Sorbitan monostearate | Span-60 (Atlas/ICI), Crill 3 | 4.7 |
| | (Croda), Nikkol SS-10 (Nikko) | |

In one or more embodiments the surface active agent is a complex emulgator in which the combination of two or more surface active agents can be more effective than a single surfactant and provides a more stable emulsion or improved foam quality than a single surfactant. For example and by way of non-limiting explanation it has been found that by choosing say two surfactants, one hydrophobic and the other hydrophilic the combination can produce a more stable emulsion than a single surfactant. Preferably, the complex emulgator comprises a combination of surfactants wherein there is a difference of about 4 or more units between the HLB values of the two surfactants or there is a significant difference in the chemical nature or structure of the two or more surfactants.

Specific non limiting examples of surfactant systems are, combinations of polyoxyethylene alkyl ethers, such as Brij 59/Brij 10; Brij 52/Brij 10; Steareth 2/Steareth 20; Steareth 2/Steareth 21 (Brij 72/Brij 721); combinations of polyoxyethylene stearates such as Myrj 52/Myrj 59; combinations of sucrose esters, such as Surphope 1816/Surphope 1807; combinations of sorbitan esters, such as Span 20/Span 80; Span 20/Span 60; combinations of sucrose esters and sorbitan esters, such as Surphope 1811 and Span 60; combinations of liquid polysorbate detergents and PEG compounds, such as Tween 80/PEG-40 stearate; methyl glucaso sequistearate; polymeric emulsifiers, such as Permulen (TR1 or TR2); liquid crystal systems, such as Arlatone (2121), Stepan (Mild RM1), Nikomulese (41) and Montanov (68) and the like.

In certain embodiments the surfactant is preferably one or more of the following: a combination of steareth-2 and steareth-21 on their own or in combination with glyceryl monostearate (GMS); in certain other embodiments the surfactant is a combination of polysorbate 80 and PEG-40 stearate. In certain other embodiments the surfactant is a combination of glyceryl monostearate/PEG 100 stearate. In certain other embodiments the surfactant is a combination of two or more of stearate 21, PEG 40 stearate, and polysorbate 80. In certain other embodiments the surfactant is a combination of two or more of laureth 4, span80, and polysorbate 80. In certain other embodiments the surfactant is a combination of two or more of GMS and ceteareth. In certain other embodiments the surfactant is a combination of two or more of steareth 21, ceteareth 20, ceteth 2 and laureth 4 In certain other embodiments the surfactant is a combination of ceteareth 20 and polysorbate 40 stearate. In certain other embodiments the surfactant is a combination of span 60 and GMS.

In certain other embodiments the surfactant is one or more of sucrose stearic acid esters, sorbitan laureth, and sorbitan stearate.

In one or more embodiments the stability of the composition can be improved when a combination of at least one non-ionic surfactant having HLB of less than 9 and at least one non-ionic surfactant having HLB of equal or more than 9 is employed. The ratio between the at least one non-ionic surfactant having HLB of less than 9 and the at least one non-ionic surfactant having HLB of equal or more than 9, is between 1:8 and 8:1, or at a ratio of 4:1 to 1:4. The resultant HLB of such a blend of at least two emulsifiers is preferably between about 9 and about 14.

Thus, in an exemplary embodiment, a combination of at least one non-ionic surfactant having HLB of less than 9 and at least one non-ionic surfactant having HLB of equal or more than 9 is employed, at a ratio of between 1:8 and 8:1, or at a ratio of 4:1 to 1:4, wherein the HLB of the combination of emulsifiers is preferably between about 5 and about 18.

In certain cases, the surface active agent is selected from the group of cationic, zwitterionic, amphoteric and ampholytic surfactants, such as sodium methyl cocoyl taurate, sodium methyl oleoyl taurate, sodium lauryl sulfate, triethanolamine lauryl sulfate and betaines.

Many amphiphilic molecules can show lyotropic liquid-crystalline phase sequences depending on the volume balances between the hydrophilic part and hydrophobic part. These structures are formed through the micro-phase segregation of two Many amphiphilic molecules can show lyotropic liquid-crystalline phase sequences depending on the volume balances between the hydrophilic part and hydrophobic part. These structures are formed through the micro-phase segregation of two incompatible components on a nanometer scale. Soap is an everyday example of a lyotropic liquid crystal. Certain types of surfactants tend to form lyotropic liquid crystals in emulsions interface (oil-in-water) and exert a stabilizing effect.

In one or more embodiments the surfactant is a surfactant or surfactant combination is capable of or which tends to form liquid crystals. Surfactants which tend to form liquid crystals may improve the quality of foams. Non limiting examples of surfactants with postulated tendency to form interfacial liquid crystals are: phospholipids, alkyl glucosides, sucrose esters, sorbitan esters.

In one or more embodiments the at least one surface active agent is liquid.

In one or more embodiments the at least one surface active agent is solid, semi solid or waxy.

It should be noted that HLB values may not be so applicable to non ionic surfactants, for example, with liquid crystals or with silicones. Also HLB values may be of lesser significance in a waterless or substantially non-aqueous environment.

In one or more embodiments the surfactant can be, a surfactant system comprising of a surfactant and a co surfactant, a waxy emulsifier, a liquid crystal emulsifier, an emulsifier which is solid or semi solid at room temperature and pressure, or combinations of two or more agents in an appropriate proportion as will be appreciated a person skilled in the art. Where a solid or semi solid emulsifier combination is used it can also comprise a solid or semi solid emulsifier and a liquid emulsifier.

In one or more embodiments, the surface-active agent includes at least one non-ionic surfactant. Ionic surfactants are known to be irritants. Therefore, non-ionic surfactants are preferred in applications including sensitive tissue such as found in most mucosal tissues, especially when they are infected or inflamed. Non-ionic surfactants alone can provide formulations and foams of good or excellent quality in the carriers and compositions.

Thus, in a preferred embodiment, the surface active agent, the composition contains a non-ionic surfactant. In another preferred embodiment the composition includes a mixture of non-ionic surfactants as the sole surface active agent. Yet, in additional embodiments, the foamable composition includes a mixture of at least one non-ionic surfactant and at least one ionic surfactant in a ratio in the range of about 100:1 to 6:1. In one or more embodiments, the non-ionic to ionic surfactant ratio is greater than about 6:1, or greater than about 8:1; or greater than about 14:1, or greater than about 16:1, or greater than about 20:1. In further embodiments, surface active agent comprises a combination of a non-ionic surfactant and an ionic surfactant, at a ratio of between 1:1 and 20:1.

In one or more embodiments, a combination of a non-ionic surfactant and an ionic surfactant (such as sodium lauryl sulphate and cocamidopropylbetaine) is employed, at a ratio of between 1:1 and 20:1, or at a ratio of 4:1 to 10:1; for example, about 1:1, about 4:1, about 8:1, about 12:1, about 16:1 and about 20:1 or at a ratio of 4:1 to 10:1, for example, about 4:1, about 6:1, about 8:1 and about 10:1.

In selecting a suitable surfactant or combination thereof it should be borne in mind that the upper amount of surfactant that may be used may be limited by the shakability of the composition. If the surfactant is non liquid, it can make the formulation to viscous or solid. This can be particularly significant if the formulation has high molecular weight, e.g., a high molecular weight PEG or polymeric agents or petroleum or if the surfactants are large. Solvents and polymeric agents which have high molecular weight and are very viscous or solid or waxy (e.g., Peg 1500, 2000, etc. or petrolatum) can exacerbate the effect of a waxy or solid surfactant on shakability or flowability In general terms, as the amount of non-liquid surfactant is increased the shakability of the formulation reduces until a limitation point is reached where the formulation becomes non shakable and unsuitable. Thus in one embodiment, an effective amount of surfactant may be used provided the formulation remains shakable. In other certain exceptional embodiments the upper limit may be determined by flowability such as in circumstances where the composition is marginally or apparently non-shakable. The formulation is sufficiently flowable to be able to flow through an actuator valve and be released and still expand to form a good quality foam.

In certain embodiments, the amount of surfactant or combination of surfactants is between about 0.05% to about 20%; between about 0.05% to about 15%. or between about 0.05% to about 10%. In a preferred embodiment the concentration of surface active agent is between about 0.2% and about 8%. In a more preferred embodiment the concentration of surface active agent is between about 1% and about 6%.

In some embodiments, it is desirable that the surface active agent does not contain a polyoxyethylene (POE) moiety, such as polysorbate surfactants, POE fatty acid esters, and POE alkyl ethers, because the active agent is incompatible with such surface active agents. For example, the active agent pimecrolimus is not stable the presence of POE moieties, yet benefits greatly from the use of dicarboxylic esters as penetration enhancers. In such cases, alternative surface active agents are employed. In an exemplary manner, POE-free surfactants include non-ethoxylated sorbitan esters, such as sorbitan monopalmitate, sorbitan monostearate, sorbitan tristearate, sorbitan monooleate, sorbitan trioleate, sorbitan monolaurate and sorbitan sesquioleate; glycerol fatty acid esters, such as glycerol monostearate and glycerol monooleate; mono-, di- and tri-esters of sucrose with fatty acids (sucrose esters), sucrose stearate, sucrose distearate sucrose palmitate and sucrose laurate; and alkyl polyglycosides, such as lauryl diglucoside.

If the composition as formulated is a substantially non shakable composition it is nevertheless possible for the formulation to be flowable to a sufficient degree to be able to flow through an actuator valve and be released and still expand to form a good quality foam. This surprising and unusual exception may be due one or more of a number of factors such as the high viscosity, the softness, the lack of crystals, the pseudoplastic or semi pseudo plastic nature of the composition and the dissolution of the propellant into the composition.

In one or more embodiments, the surface-active agent includes mono-, di- and tri-esters of sucrose with fatty acids (sucrose esters), prepared from sucrose and esters of fatty acids or by extraction from sucro-glycerides. Suitable sucrose esters include those having high monoester content.

Phase Inversion and Tension

Phase inversion is a factor in the preparation and stabilization of emulsions and can be both an aid and a detriment. Phase inversion involves the change of emulsion type from o/w to w/o or vice versa. Prior to phase inversion occurring there is a tension in the emulsion which if destabilized or driven will lead to phase inversion and if controlled or ameliorated or dissipated will result in a more stable emulsion. The occurrence of phase inversion during preparation can be a sign of instability. If controlled, it can result in a finer product but if due to other factors after the emulsion was prepared it can cause problems. Inversion can occur by for example adding calcium chloride to an o/w emulsion stabilized with sodium stearate to form calcium stearate. Inversion can also occur as the product of changes to the phase-volume ratio. For example if a small amount of water is added to surfactant mixed with oil and agitated aw/o emulsion is formed As the amount of water added is gradually increased a point will be reached where the water and emulsifier envelop the oil as small droplets to form an o/w emulsion. The amount of each ingredient including the surfactants will have their part to play in the phenomenon.

Substantially Alcohol-Free

According to one or more embodiments, the foamable composition is substantially alcohol-free, i.e., free of short chain alcohols. Short chain alcohols, having up to 5 carbon atoms in their carbon chain skeleton and one hydroxyl group, such as ethanol, propanol, isopropanol, butaneol, iso-butaneol, t-butaneol and pentanol, are considered less desirable solvents or polar solvents due to their skin-irritating effect. Thus, the composition is substantially alcohol-free and includes less than about 5% final concentration of lower alcohols, preferably less than about 2%, more preferably less than about 1%.

Substantially Non Aqueous

In certain cases, the active agent degrades in the presence of water, and therefore, in such cases the present of water in the composition is not desirable. Thus, in certain preferred embodiments, the composition is substantially non-aqueous. The term “substantially non-aqueous” or “substantially waterless” is intended to indicate that the composition has a water content below about 5%, preferably below about 2%, such as below about 1.5%. In certain other preferred embodiments the composition is non aqueous or waterless.

By non aqueous or waterless is meant that the composition contains no or substantially no, free or unassociated or absorbed water. It will be understood by a person of the art that the waterless solvents and substances miscible with them can be hydrophilic and can contain water in an associated or unfree or absorbed form and may absorb water from the atmosphere and the ability to do so is its hygroscopic water capacity. It is intended that essentially non-aqueous formulations are included within its scope such that the formulations may have present a small amount of water. In some embodiments the composition ingredients are pretreated to reduce, remove or eliminate any residual or associated or absorbed water.

Shakability

‘Shakability’ means that the composition contains some or sufficient flow to allow the composition to be mixed or remixed on shaking. That is, it has fluid or semi fluid properties. In some very limited cases possibly aided by the presence of silicone it may exceptionally be possible to have a foamable composition which is flowable but not apparently shakable.

Breakability

A breakable foam is one that is thermally stable, yet breaks under sheer force.

The breakable foam, described according to one or more embodiments, is not “quick breaking”, i.e., it does not readily collapse upon exposure to body temperature environment. Sheer-force breakability of the foam is clearly advantageous over thermally induced breakability, since it allows comfortable application and well directed administration to the target area.

Modulating Agent

The term modulating agent is used to describe an agent which can improve the stability of or stabilize a foamable carrier or composition and or an active agent by modulating the effect of a substance or residue present in the carrier or composition.

In one or more embodiments the modulating agent is used in a water in oil or oil in water emulsion. In one or more other embodiments the modulating agent is used in a unique waterless emulsion.

In certain embodiments, the substance or residue may for example be acidic or basic and potentially alter pH in an emulsion environment or it may be one or more metal ions which may act as a potential catalyst in an emulsion environment.

In certain other embodiments, the substance or residue may for example be acidic or basic and potentially alter an artificial pH in a waterless or substantially non aqueous environment or it may be one or more metal ions which may act as a potential catalyst in a waterless or substantially non aqueous environment.

In one or more embodiments, the modulating agent is used to describe an agent which can affect pH in an aqueous solution. The agent can be any of the known buffering systems used in pharmaceutical or cosmetic formulations as would be appreciated by a man of the art. It can also be an organic acid, a carboxylic acid, a fatty acid an amino acid, an aromatic acid, an alpha or beta hydroxyl acid an organic base or a nitrogen containing compound.

In one or more further embodiments, the modulating agent is used to describe an agent, which is a chelating or sequestering or complexing agent that is sufficiently soluble or functional in the solvent to enable it to “mop up” or “lock” metal ions.

In an embodiment, modulating agent is used to describe an agent which can effect pH in an aqueous solution the term modulating agent more particularly means an acid or base or buffer system or combinations thereof, which is introduced into or is present in and acts to modulate the ionic or polar characteristics and any acidity or basicity balance of an emulsion carrier, composition, foamable carrier or foamable composition or resultant foam.

In other embodiments, modulating agent is used to describe an agent which can effect pH in an aqueous solution the term modulating agent more particularly means an acid or base or buffer system or combinations thereof, which is introduced into or is present in and acts to modulate the ionic or polar characteristics and any acidity or basicity balance of a waterless or substantially non aqueous carrier, composition, foamable carrier or foamable composition or resultant foam.

The substance or residue can be introduced into the formulation from any one or more of the ingredients, some of which themselves may have acidic or basic properties. For example the polymer or solvent may contain basic residues in which case it may be desirable or beneficial to add an acid. Alternatively the surfactant may contain some acid residues in which case the addition of a base may be desirable and beneficial. In some cases more than one ingredient may contain residues which may ameliorate or compound their significance. For example if one ingredient provided weak acid residues and another stronger acid residues the pH in an emulsion environment (or artificial pH in a waterless environment) should be lower. In contrast if one residue was acid and the other basic the net effect in the formulation maybe significantly reduced. In some circumstances the active ingredient may favor an acidic pH or more significantly may need to be maintained at a certain acidic pH otherwise it may readily isomerize, chemically react or breakdown, in which case introducing acidic components such as an acidic polymer might be of help. In an embodiment sufficient modulating agent is added to achieve a pH in which the active agent is preferably stable. In another embodiment sufficient modulating agent is added to achieve an artificial pH in which the active agent is preferably stable.

The terms pH, pKa, and pKb, buffers and the like are used in classical measurements of an aqueous solution. Such measurements are artificial in a waterless environment. Nevertheless, reference to and description below of such terms are made for convenience and clarity, since such terms are well defined and understood with reference to aqueous solutions and further due to the lack of an appropriate uniform way of describing and identifying the artificial or virtual pH, pK etc in a waterless environment in relation to the present invention. Although predictions of artificial pH can be made using dilution techniques of measurements of waterless formulations diluted in water they are formulation sensitive and specific and have to be carefully calibrated with complex formulas.

Waterless medium can be polar and protic yet it does not conform to classical ionic behavior.

A buffer, as defined by Van Slyke [Van Slyke, J. Biol. Chem. 52, 525 (1922)], is “a substance which by its presence in solution increases the amount of acid or alkali that must be added to cause unit change in pH.”

A buffer solution is a solution of a definite pH made up in such a way that this pH alters only gradually with the addition of alkali or acid. Such a solution consists of a solution of a salt of the week acid in the presence of the three acid itself. The pH of the solution is determined by the dissociation equilibrium of the free acid.

An acid can be a strong acid or a weak acid. A strong acid is an acid, which is a virtually 100% ionized in solution. In contrast, a week acid is one which does not ionize fully. When it is dissolved in water. The lower the value for pKa, the stronger is the acid and likewise, the higher the value for pKa the weaker is the acid.

A base can be a strong base or a weak base. A strong base is something, which is fully ionic with 100% hydroxide ions. In contrast, a weak base is one which does not convert fully into hydroxide ions in solution. The lower the value for pKb, the stronger is the base and likewise, the higher the value for pKb the weaker is the base.

In one or more embodiments, the modulating agent comprises an organic compound.

In one or more preferred embodiments the chelating agent is selected from the group consisting of ethylenediaminetetraacetic acid (EDTA), diethylenetriaminepentaacetic acid (DTPA), hydroxyethylenediaminetriacetic acid (HEDTA), nitrilotriacetic acid (NTA), O,O′-bis(2-aminoethyl)ethyleneglycol-N,N,N′,N′-tetraacetic acid (EGTA), trans-1,2-diaminocyclohexane-N,N,N′,N′-tetraacetic acid (CyDTA) or a pharmaceutically acceptable salt thereof (normally as a sodium salt), more preferably EDTA, HEDTA and their salts; most preferably EDTA and its salts.

In one or more embodiments, a preferred non limiting example of the chelating agent is EDTA. Typically, the chelating and sequestering agent is present in the composition at a level of up to about 5.0%, preferably 1.0 percent, by weight, of the composition.

In one or more embodiments, the modulating agent may also be a preservative or an antioxidant or an ionization agent. Any preservative, antioxidant or ionization agents suitable for pharmaceutical or cosmetic application may be used. Non limiting examples of antioxidants are tocopherol succinate, propyl galate, butylated hydroxy toluene and butyl hydroxy anisol. Ionization agents may be positive or may be negative depending on the environment and the active agent or composition that is to be protected. Ionization agents may for example act to protect or reduce sensitivity of active agents. Non limiting examples of positive ionization agents are benzyl conium chloride, and cetyl pyridium chloride. Non limiting examples of negative ionization agents are sodium lauryl sulphate, sodium lauryl lactylate and phospholipids.

Hydrophobic Solvent

Optionally, the foamable carrier further contains at least one hydrophobic solvent. The identification of a “hydrophobic solvent”, as used herein, is not intended to characterize the solubilization capabilities of the solvent for any specific active agent or any other component of the foamable composition. Rather, such information is provided to aid in the identification of materials suitable for use as a part in the foamable compositions described herein.

A “hydrophobic solvent” as used herein refers to a material having solubility in distilled water at ambient temperature of less than about 1 gm per 100 mL, more preferable less than about 0.5 gm per 100 mL, and most preferably less than about 0.1 gm per 100 mL.

In one or more embodiments, the hydrophobic organic carrier is an oil, such as mineral oil, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated lanolin alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, unsaturated or polyunsaturated oils, such as olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils; essential oils; and silicone oils, such as dimethicone, cyclomethicone, polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers.

Foam Adjuvant

Optionally, a foam adjuvant is included in the foamable carriers to increase the foaming capacity of surfactants and/or to stabilize the foam. In one or more embodiments, the foam adjuvant agent includes fatty alcohols having 15 or more carbons in their carbon chain, such as cetyl alcohol and stearyl alcohol (or mixtures thereof). Other examples of fatty alcohols are arachidyl alcohol (C20), behenyl alcohol (C22), 1-triacontanol (C30), as well as alcohols with longer carbon chains (up to C50). Fatty alcohols, derived from beeswax and including a mixture of alcohols, a majority of which has at least 20 carbon atoms in their carbon chain, are especially well suited as foam adjuvant agents. The amount of the fatty alcohol required to support the foam system is inversely related to the length of its carbon chains. Foam adjuvants, as defined herein are also useful in facilitating improved spreadability and absorption of the composition.

In one or more embodiments, the foam adjuvant agent includes fatty acids having 16 or more carbons in their carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof. As for fatty alcohols, the amount of fatty acids required to support the foam system is inversely related to the length of its carbon chain.

Optionally, the carbon atom chain of the fatty alcohol or the fatty acid may have at least one double bond. A further class of foam adjuvant agent includes a branched fatty alcohol or fatty acid. The carbon chain of the fatty acid or fatty alcohol also can be substituted with a hydroxyl group, such as 12-hydroxy stearic acid.

Additional Components

In an embodiment, a composition includes one or more additional components. Such additional components include but are not limited to anti perspirants, anti-static agents, buffering agents, bulking agents, conservational agents, chelating agents, cleansers, colorants, conditioners, deodorants, diluents, dyes, emollients, fragrances, hair conditioners, humectants, pearlescent aids, perfuming agents, permeation enhancers, pH-adjusting agents, preservatives, protectants, skin penetration enhancers, softeners, solubilizers, sunscreens, sun blocking agents, sunless tanning agents, viscosity modifiers, antioxidants like flavonoids and phenolics. As is known to one skilled in the art, in some instances a specific additional component may have more than one activity, function or effect.

In an embodiment, the additional component is a pH adjusting agent or a buffering agent. Suitable buffering agents include but are not limited to acetic acid, adipic acid, calcium hydroxide, citric acid, glycine, hydrochloric acid, lactic acid, magnesium aluminometasilicates, phosphoric acid, sodium carbonate, sodium citrate, sodium hydroxide, sorbic acid, succinic acid, tartaric acid, and derivatives, salts and mixtures thereof.

In an embodiment, the additional component is an emollient. Suitable emollients include but are not limited to mineral oil, lanolin oil, coconut oil, cocoa butter, olive oil, aloe vera extract, jojoba oil, castor oil, fatty acids, fatty alcohols, diisopropyl adipate, hydroxybenzoate esters, benzoic acid esters of C9 to C15 alcohols, isononyl iso-nonanoate, silicone oils, polyethers, C12 to C15 alkyl benzoates, oleic acid, stearic fatty acid, cetyl alcohols, hexadecyl alcohol, dimethyl polysiloxane, polyoxypropylene cetyl ether, polyoxypropylene butyl ether, and derivatives, esters, salts and mixtures thereof.

In an embodiment, the additional component is a humectant, a substance that helps retain moisture and also prevents rapid evaporation. Suitable humectants include but are not limited to guanidine, urea, glycolic acid, glycolate salts, ammonium glycolate, quaternary alkyl ammonium glycolate, lactic acid, lactate salts, ammonium lactate, quaternary alkyl ammonium lactate, aloe vera, aloe vera gel, allantoin, urazole, alkoxylated glucose, hyaluronic acid, lactamide monoethanolamine, acetamide monoethanolamine and derivatives, esters, salts and mixtures thereof. Other non limiting examples are propylene glycol, propylene glycol derivatives, glycerin, hydrogenated starch hydrosylate, hydrogenated lanolin, lanolin wax, D manitol, sorbitol, sodium 2-pyrrolidone-5-carboxylate, sodium lactate, sodium PCA, soluble collagen, dibutyl phthalate, and gelatin. Additional examples may be found in the Handbook of Pharmaceutical Additives published by Gower.

In an embodiment, the additional component is a moisturizer, is a substance that helps retain moisture or add back moisture to the skin. Examples are allantoin, petrolatum, urea, lactic acid, sodium PCV, glycerin, shea butter, caprylic/capric/stearic triglyceride, candelilla wax, propylene glycol, lanolin, hydrogenated oils, squalene, sodium hyaluronate and lysine PCA. Other examples may be found in the Handbook of Pharmaceutical Additives published by Gower.

Pharmaceutical compositions may in one or more embodiments usefully comprise in addition a heumectant or a moisturizer or combinations thereof.

In an embodiment the additional component is a conservational agent, which can be a preservative, quasi preservative or a modulating agent or combinations thereof that can substantially conserve or stabilize the vitamin. By substantially conserve or stabilize the vitamin is meant that it acts to inhibit, restrain or delay breakdown or reaction of the vitamin in the carrier or foamable composition so that most of the vitamin remains available for use in the foam formulation.

In an embodiment, the additional component is a preservative. Suitable preservatives include but are not limited to C12 to C15 alkyl benzoates, alkyl p-hydroxybenzoates, aloe vera extract, ascorbic acid, benzalkonium chloride, benzoic acid, benzoic acid esters of C9 to C15 alcohols, butylated hydroxytoluene, castor oil, cetyl alcohols, chlorocresol, citric acid, cocoa butter, coconut oil, diazolidinyl urea, diisopropyl adipate, dimethyl polysiloxane, DMDM hydantoin, ethanol, fatty acids, fatty alcohols, hexadecyl alcohol, hydroxybenzoate esters, iodopropynyl butylcarbamate, isononyl iso-nonanoate, jojoba oil, lanolin oil, methylparaben, mineral oil, oleic acid, olive oil, polyethers, polyoxypropylene butyl ether, polyoxypropylene cetyl ether, potassium sorbate, silicone oils, sodium propionate, sodium benzoate, sodium bisulfite, disodium metabisulfite, sorbic acid, stearic fatty acid, vitamin E, vitamin E acetate and derivatives, esters, salts and mixtures thereof.

In an embodiment the preservative comprises an antioxidant.

In one or more embodiments flavonoids, as strong antioxidants, may act as a quasi preservative of a vitamin in a formulation by, without being bound by any theory, being more reactive in a formulation than the vitamin or derivative and thereby sparing the vitamin or derivative from reaction. As certain flavonoids can be water soluble and certain others hydrophobic, they can be selected to offer protection both in non-aqueous an in aqueous formulations. In a preferred embodiment a combination of one or more water soluble flavonoids with one or more hydrophobic flavonoids may be used. For example, quercetin combined with rosmarinic acid.

In an embodiment the preservative comprises at least disodium metabisulfite or a derivative. In a further embodiment it is used in combination with another preservative, and or a modulating agent and or a flavonoid

Sodium Metabisulfite (Disodium Metabisulfite) and Derivatives

Sodium metabisulfite is used as an antioxidant in (primarily acidic) pharmaceutical formulations, at concentrations of 0.01-1.0% w/v., preparations and can also be used as a preservative having some antimicrobial activity although for alkaline preparations, sodium sulfite is usually preferred

On exposure to air and moisture, sodium metabisulfite is slowly oxidized to sodium sulfate In water, sodium metabisulfite is immediately converted to sodium (Na⁺) and bisulfite (HSO⁻₃) ions. Aqueous sodium metabisulfite solutions also decompose in air, and are more susceptible in the presence of dextrose.

Sodium metabisulfite reacts with sympathomimetics and other drugs that are ortho- or para-hydroxybenzyl alcohol derivatives to form sulfonic acid derivatives possessing little or no pharmacological activity is incompatible with chloramphenicol and inactivates cisplatin in solution.

Sodium metabisulfite is used as an antioxidant at low pH, sodium bisulfite at intermediate pH, and sodium sulfite at higher pH values.

In an embodiment the preservative comprises EDTA.

In an embodiment the preservative comprises sharonmix 824.

In an embodiment, the additional component is a wax such as bees wax, a wax like substance like stearic acid or a liquid wax like jojoba oil, isostearic acid or oleyl alcohol and similar fatty acids and fatty alcohols.

In an embodiment of the present invention, the additional component is a skin penetration enhancer. Suitable skin penetration enhancers include but are not limited to acetone, acyl lactylates, acyl peptides, acylsarcosinates, alkanolamine salts of fatty acids, alkyl benzene sulphonates, alkyl ether sulphates, alkyl sulphates, anionic surface-active agents, benzyl benzoate, benzyl salicylate, butan-1,4-diol, butyl benzoate, butyl laurate, butyl myristate, butyl stearate, cationic surface-active agents, citric acid, cocoamidopropylbetaine, decyl methyl sulfoxide, decyl oleate, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, di(2-hydroxypropyl)ether, diisopropyl adipate, diisopropyl sebacate, N,N-dimethyl acetamide, dimethyl azelate, N,N-dimethyl formamide, 1,5-dimethyl-2-pyrrolidone, dimethyl sebacate, dimethyl sulphoxide, dioctyl adipate, dioctyl azelate, dioctyl sebacate, 1,4 dioxane, 1-dodecylazacyloheptan-2-one, dodecyl dimethyl amine oxides, ethyl caprate, ethyl caproate, ethyl caprylate, 2-ethyl-hexyl pelargonate, ethyl-2-hydroxypropanoate, ethyl laurate, ethyl myristate, 1-ethyl-2-pyrrolidone, ethyl salicylate, hexyl laurate, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hexan-2,5-diol, khellin, lamepons, lauryl alcohol, maypons, metal salts of fatty acids, methyl nicotinate, 2-methyl propan-2-ol, 1-methyl-2-pyrrolidone, 5-methyl-2-pyrrolidone, methyl taurides, miranol, nonionic surface-active agents, octyl alcohol, octylphenoxy polyethoxyethanol, oleic ethanolamide, pleyl alcohol, pentan-2,4-diol, phenoxyethanol, phosphatidyl choline, phosphine oxides, polyalkoxylated ether glycollates, poly(diallylpiperidinium chloride), poly(dipropyldiallylammonium chloride), polyglycerol esters, polyoxyethylene lauryl ether, polyoxy:polyoxyethylene stearate, polyoxypropylene 15 stearyl ether, poly(vinyl pyridinium chloride), propan-1-ol, propan-2-ol, propylene glycol dipelargonate, pyroglutamic acids, 2-pyrrolidone, pyruvic acids, Quaternium 5, Quaternium 18, Quaternium 19, Quaternium 23, Quaternium 31, Quaternium 40, Quaternium 57, quartenary amine salts, quaternised poly (dimethylaminoethylmethacrylate), quaternised poly (vinyl alcohol), sapamin hydrochloride, sodium cocaminopropionate, sodium dioctyl sulphonsuccinate, sodium laurate, sodium lauryl ether sulphate, sodium lauryl sulphate, sugar esters, sulphosuccinate, tetrahydrofuran, tetrahydrofurfural alcohol, transcutol, triethanolamine dodecyl benzene sulphonate, triethanolamine oleate, urea, water and derivatives, esters, salts and mixtures thereof.

A “penetration enhancer,” can be an organic solvent, typically soluble in both water and oil. Further, examples of penetration enhancer include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, hexylene glycol, other glycols, sulfoxides, such as dimethylsulfoxide (DMSO), dimethylformanide, methyl dodecyl sulfoxide, dimethylacetamide, dimethylisosorbide, monooleate of ethoxylated glycerides (with 8 to 10 ethylene oxide units), azone (1-dodecylazacycloheptan-2-one), 2-(n-nonyl)-1,3-dioxolane, esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid; lactam compounds, such as azone; alkanols, such as dialkylamino acetates, and admixtures thereof.

According to one or more embodiments, the penetration enhancer is a polyethylene glycol (PEG) or PEG derivative that is liquid at ambient temperature.

In one or more embodiments, the foamable composition includes a potent solvent, in addition to or in place of one of the hydrophobic solvents, polar solvents or emollients of the composition. A potent solvent is a solvent other than mineral oil that solubilizes a specific active agent substantially better than a hydrocarbon solvent such as mineral oil or petrolatum. For example, a potent solvent solubilizes the active agent 5 fold better than a hydrocarbon solvent; or even solubilizes the active agent 10-fold better than a hydrocarbon solvent.

In one or more embodiments, the composition includes at least one active agent in a therapeutically effective concentration; and at least one potent solvent in a sufficient amount to substantially solubilize the at least one active agent in the composition. The term “substantially soluble” means that at least 95% of the active agent has been solubilized, i.e., 5% or less of the active agent is present in a solid state. In one or more embodiments, the concentration of the at least one potent solvent is more than about 40% of the at least one solvent of the composition; or even more than about 60%.

Non-limiting examples of pairs of active agent and potent solvent include: Betamethasone valerate: Practically insoluble in mineral oil (<0.01%); soluble more than 1% in glycofurol; Hydrocortisone butyrate: Practically insoluble in mineral oil (<0.01%); soluble more than 1% in glycofurol; Metronidazole: Practically insoluble in mineral oil (<0.01%); soluble more than 1% in dimethyl isosorbide; Ketoconazole: Practically insoluble in mineral oil (<0.01%); soluble more than 1% in glycofurol, propylene glycol and dimethyl isosorbide; Mupirocin: Practically insoluble in mineral oil (<0.01%); soluble more than 1% in glycofurol, hexylene glycol, dimethyl isosorbide, propylene glycol and polyethylene glycol 400 (PEG 400); Meloxicam, a nonsteroidal anti-inflammatory agent: Practically insoluble in mineral oil (<0.001%); soluble in propylene glycol: 0.3 mg/mL; and in PEG 400: 3.7 mg/mL; and Progesterone: Practically insoluble in mineral oil (<0.001%); soluble in PEG 400: 15.3 mg/mL.

A non-limiting exemplary list of solvents that can be considered as potent solvents includes polyethylene glycol, propylene glycol, hexylene glycol, butanediols and isomers thereof, glycerol, benzyl alcohol, DMSO, ethyl oleate, ethyl caprylate, diisopropyl adipate, dimethylacetamide, N-methylpyrrolidone, N-hydroxyethylpyrrolidone, polyvinylpyrrolidone, isosorbide derivatives, such as dimethyl isosorbide, glycofurol and ethoxydiglycol (transcutol) and laurocapram.

The use of a potent solvent in a foam composition provides an improved method of delivering poorly soluble therapeutic agents to a target area. It is known that low drug solubility results in poor bioavailability, leading to decreased effectiveness of treatment. Foam compositions, for which the solvent includes a potent solvent, increase the levels of the active agent in solution and thus, provide high delivery and improved therapy.

Potent solvents, as defined herein, are usually liquid. Formulations comprising potent solvents and active agents are generally disadvantageous as therapeutics, since their usage involves unwanted dripping and inconvenient method of application; resulting in inadequate dosing. Surprisingly, the foams, which are drip-free, provide a superior vehicle for such active agents, enabling convenient usage and accurate effective dosing.

In one or more embodiments the present invention the foamable pharmaceutical composition may additionally include a mixture of two or more of the solvents selected from the group of hydrophobic solvents, silicone oils, emollients, polar solvents and potent solvents in an appropriate proportion as would be appreciated to a person skilled in the art.

In one or more embodiments, PPG alkyl ether may act as a potent solvent.

Microemulsions and Nanoemulsions

Microemulsions and nanoemulsion are translucent (or transparent) dispersions of oil and water. Compared to conventional emulsions, microemulsions and nanoemulsion are more thermodynamically stable, making them a favorable vehicle for pharmaceutical compositions, which have to maintain stability for long periods of time. Microemulsions are used, for example, for controlled release of pharmaceutical agents. In contrast to microemulsions they are in a meta-stable state having very fine oil in water dispersions with diameters of <100 nm with good sensorial and biophysical properties such as improved penetration and hydrating power respectively. They and a method of manufacture are more particularly described in US2006/0233721 which is incorporated herein by reference in its entirety. As will be appreciated by a man of the art the methodology may be adapted according to the type of carrier composition.

In one or more embodiments the composition comprises microemulsions or nano-emulsions in which the stability and or delivery/penetration of the vitamins and or flavonoids and or active ingredients are modified or improved when compared to that seen with regular emulsions.

In one or more embodiments the composition comprises microemulsions or nano-emulsions and the vitamins and or flavonoids and or active ingredients are loaded in microsponges which are dispersed in the composition.

Aging

In order to project the potential shelf life and stability of the compositions and their ingredients particularly active or benefit agents the compositions can subjected to a number of tests, including centrifugation to look for resistance to creaming, phase separation; one or more freeze thaw cycles, standing at room and higher temperatures as an indicator of resistance to aging.

Propellants

Suitable propellants include volatile hydrocarbons such as butane, propane, isobutane and fluorocarbon gases, or mixtures thereof.

The propellant makes up about 5-25 wt % of the foamable composition. The propellants are used to generate and administer the foamable composition as a foam. The total composition including propellant, foamable compositions and optional ingredients is referred to as the foamable composition.

Alcohol and organic solvents render foams inflammable. It has been surprisingly discovered that fluorohydrocarbon propellants, other than chloro-fluoro carbons (CMCs), which are non-ozone-depleting propellants, are particularly useful in the production of a non-flammable foamable composition. A test according to European Standard prEN 14851, titled “Aerosol containers—Aerosol foam flammability test” revealed that compositions containing an organic carrier that contains a hydrophobic organic carrier and/or a polar solvent, which are detected as inflammable when a hydrocarbon propellant is used, become non-flammable, while the propellant is an HFC propellant.

Such propellants include, but are not limited to hydrofluorocarbon (HFC) propellants, that contain no chlorine atoms, and as such, falls completely outside concerns about stratospheric ozone destruction by chlorofluorocarbons or other chlorinated hydrocarbons. Exemplary non-flammable propellants according to this aspect of the invention include propellants made by DuPont under the registered trademark Dymel, such as 1,1,1,2 tetrafluorethane (Dymel 134), and 1,1,1,2,3,3,3 heptafluoropropane (Dymel 227), 1,1, difluoro ethane (Dymel 152) and 1,1,1,3,3,3 hexafluoropropane. HFCs possess Ozone Depletion Potential of 0.00 and thus, they are allowed for use as propellant in aerosol products.

Notably, the stability of foamable emulsions including HFC as the propellant can be improved in comparison with the same composition made with a hydrocarbon propellant.

In one or more embodiments foamable compositions comprise a combination of a HFC and a hydrocarbon propellant such as n-butane or mixtures of hydrocarbon propellants such as propane, isobutane and butane.

In one or more embodiments the propellant when added to the pre foam foamable composition can dissolve in the formulation to improve flowability and shakability and reduce viscosity. An additional effect of the propellants particularly when filled under vacuum may be that, without being bound by any theory, they can compete with, replace, or dilute out the small or tiny amounts of remaining oxygen so that remaining oxygen is rendered substantially ineffective. In this connection, any remaining oxygen in the formulation may have an increased tendency to evaporate to occupy the canister dead space compared to the hydrocarbon propellant. In consequence of these factors, the oxidation potential of the composition is reduced. This is primarily facilitated and assisted by filling under vacuum; and further, optionally, by prior production under an inert gas and subsequently under vacuum. For formulations with a hydrophobic phase and some solubility of the vitamin in the hydrophobic phase, the hydrophobic propellant may be to an extent, without being bound by any theory, directly protective of the vitamin, and even where the vitamin is water soluble it may still offer a protective synergy. In consequence, the foam formulation may be less susceptible to oxidative breakdown of the vitamin. To the extent a flavonoid is also present which is more reactive to oxidation than the vitamin then it is postulated that the flavonoid will react whilst allowing the vitamin to remain unoxidized. Likewise the presence of another conservative agent can further help preserve the vitamin.

Hygroscopic Property of the (Substantially) Non-Aqueous Composition

A hydroscopic substance is a substance that absorbs water readily from its surroundings. Microorganisms require water to grow and reproduce, and such water requirements are best defined in terms of water activity of the substrate. The water activity of a solution is expressed as Aw=P/Po, where P is the water vapor pressure of the solution and Po is the vapor pressure of pure water at the same temperature. The of a hygroscopic substance to a solution in which a vitamin is present containing some water will have the effect of lowering the Aw, with a postulated consequent effect of reducing the availability of water to facilitate breakdown of the vitamin and in parallel effect upon cell growth of microorganisms, if present. Also, where the composition is substantially non aqueous, it is postulated that the water that is present in the composition will be at least to the most part be associated with hygroscopic solvent so that it will have a consequent effect of reducing the availability of water to facilitate breakdown of the vitamin Every microorganism has a limiting Aw, below which it will not grow, e.g., for streptococci, klebsiella spp., escherichia coli, clostridium perfringens, and pseudomonas spp. the Aw value is 0.95. Staphylococcus aureus is most resistant and can proliferate with an Aw as low as 0.86.

The water activity of a product can be determined from the relative humidity of the air surrounding the sample when the air and the sample are at equilibrium. Measurement is performed by placing a sample in an enclosed space where this equilibrium can take place. Once this occurs, the water activity of the sample and the relative humidity of the air are equal. The measurement taken at equilibrium is called an equilibrium relative humidity or ERH. The relationship between the water activity and ERH is in accordance with the following formula:
Aw=ERH/100

Various types of water activity instruments are commercially available. One exemplary instrument uses chilled-mirror dewpoint technology while other instruments measure relative humidity with sensors that change electrical resistance or capacitance.

Polyols, PEGs and other polar solvents have a great affinity for water, and as such, they exhibit hygroscopic properties. The concentration of the polyol, the PEG and/or other polar solvents determines the Aw of the carrier. In one or more embodiments, the polyols, the PEG and/or the secondary polar solvent is contained in the composition at a sufficient concentration to provide an Aw value of the hygroscopic carrier of less than 0.9. In other embodiments, the concentration of the polyol, the PEG and/or secondary polar solvent in the composition is selected to provide a Aw value selected from the ranges of (1) about 0.8 and about 0.9; (2) about 0.7 and about 0.8; and (3) less than about 0.7.

As such, a composition containing a polyol, a PEG with or without a secondary polar solvent can be used as topical treatment of superficial infectious conditions.

The advantage of providing a hygroscopic composition in a pressurized packaging presentation is readily perceived. The usage of all other presentations, such as solutions, creams, lotions, ointments and the like involves repeated opening of the package closure, resulting in absorption of water from the surrounding environment and a subsequent elevation of the Aw (thus lowering the hygroscopicity of the product, and therefore decreasing its anti-infective potential. By contrast, a pressurized packaging does not allow for any humidity to be absorbed by the preparation, and therefore, the hygroscopic character of the composition cannot be damaged.

In one or more embodiments, the presence of a hygroscopic solvent in the formulation helps to ameliorate, reduce or prevent breakdown of a vitamin into a non active form. In a preferred embodiment, the formulation containing hygroscopic solvent is substantially non aqueous. In another preferred embodiment, the formulation containing hygroscopic solvent is non aqueous, such that any water which might be absorbed from the atmosphere during preparation will be closely associated with the hygroscopic solvent such that it is not expected to be available, to facilitate significant breakdown of vitamin.

In one or more embodiments, the hygroscopic composition further contains an anti-infective agent, selected from the group of an antibiotic agent, an antibacterial agent, an antifungal agent, an agent that controls yeast, an antiviral agent and an antiparasitic agent. Combining the anti-infective effect of a hygroscopic composition, which acts through a dehydration mechanism, with an additional anti-infective agent that acts through alternate mechanisms, results in a synergistic effect and consequently higher success rate of such treatment.

Composition and Foam Physical Characteristics and Advantages

A pharmaceutical or cosmetic composition manufactured using the foamable carrier is very easy to use. When applied onto the afflicted body surface of mammals, i.e., humans or animals, it is in a foam state, allowing free application without spillage. Upon further application of a mechanical force, e.g., by rubbing the composition onto the body surface, it freely spreads on the surface and is rapidly absorbed.

The foamable composition is stable, having an acceptable shelf-life of at least one year, or preferably, at least two years at ambient temperature, as revealed in accelerated stability tests. Organic carriers and propellants tend to impair the stability of emulsions and to interfere with the formation of stable foam upon release from a pressurized container. It has been observed, however, that the foamable compositions according to the present invention are surprisingly stable. Following accelerated stability studies, they demonstrate desirable texture; they form fine bubble structures that do not break immediately upon contact with a surface, spread easily on the treated area and absorb quickly.

The composition should also be free flowing, to allow it to flow through the aperture of the container, e.g., and aerosol container, and create an acceptable foam.

Foam quality can be graded as follows:

Grade E (excellent): very rich and creamy in appearance, does not show any bubble structure or shows a very fine (small) bubble structure; does not rapidly become dull; upon spreading on the skin, the foam retains the creaminess property and does not appear watery.

Grade G (good): rich and creamy in appearance, very small bubble size, “dulls” more rapidly than an excellent foam, retains creaminess upon spreading on the skin, and does not become watery.

Grade FG (fairly good): a moderate amount of creaminess noticeable, bubble structure is noticeable; upon spreading on the skin the product dulls rapidly and becomes somewhat lower in apparent viscosity.

Grade F (fair): very little creaminess noticeable, larger bubble structure than a “fairly good” foam, upon spreading on the skin it becomes thin in appearance and watery.

Grade P (poor): no creaminess noticeable, large bubble structure, and when spread on the skin it becomes very thin and watery in appearance.

Grade VP (very poor): dry foam, large very dull bubbles, difficult to spread on the skin.

Topically administrable foams are typically of quality grade E or G, when released from the aerosol container. Smaller bubbles are indicative of more stable foam, which does not collapse spontaneously immediately upon discharge from the container. The finer foam structure looks and feels smoother, thus increasing its usability and appeal.

As further aspect of the foam is breakability. The breakable foam is thermally stable, yet breaks under sheer force. Sheer-force breakability of the foam is clearly advantageous over thermally induced breakability. Thermally sensitive foams immediately collapse upon exposure to skin temperature and, therefore, cannot be applied on the hand and afterwards delivered to the afflicted area.

The foam has several advantages, when compared with hydroalcoholic foam compositions, such as described in WO 2004/071479:
(1) Breakability. The foam is thermally stable. Unlike hydroalcoholic foam compositions of the prior art, the foam is not “quick breaking”, i.e., it does not readily collapse upon exposure to body temperature environment. Sheer-force breakability of the foam is clearly advantageous over thermally induced breakability, since it allows comfortable application and well directed administration to the target area.
(2) Skin drying and skin barrier function. Short chain alcohols are known to dry the skin and impair the integrity of the skin barrier. By contrast, including a film forming agent in the composition foes not cause unwanted skin barrier damage.
(3) Irritability. Due to the lack of alcohol and improvement in skin barrier function, skin irritability is eliminated.

Another property of the foam is specific gravity, as measured upon release from the aerosol can. Typically, foams have specific gravity of less than 0.12 g/mL; or less than 0.10 g/mL; or less than 0.08 g/mL, depending on their composition and on the propellant concentration.

Pharmaceutical Composition

The foamable composition is an ideal vehicle for vitamins, flavonoids, active pharmaceutical ingredients and active cosmetic ingredients. It is also suitable for using microsponges to deliver, one or more of vitamins, flavonoids, active pharmaceutical ingredients and active cosmetic ingredients to a target in a controlled way. In the context herein, active pharmaceutical ingredients and active cosmetic ingredients including vitamins are collectively termed “active agent” or “active agents”. A foamable composition, comprising an active agent has the following advantages:
1. The foamable composition provides a preferred solvent for active agents, particularly water-insoluble agents.
2. The inclusion of a polyol and/or a PEG and a secondary polar solvent in the foamable composition facilitates a co-solvent effect, resulting increased concentrations of soluble active agent in the dosage form, thus facilitating enhanced skin penetration of the active agent. In many cases, increased penetration is positively correlated with improved clinical outcome. In certain case, attaining an increased drug penetration into the target site of action enables a decrease of treatment frequency, for example, from twice or three times daily to once daily.
3. Polyols and PEGs; and combinations of a polyol and/or PEG with a secondary polar solvent are known as skin penetration enhancers, thus, increasing drug residence in the target area and increasing clinical efficacy, as detailed above.
4. The fact that the composition contains no water, or up to 25% and more preferably up to 10% water minimizes the probability of degradation of water-sensitive active agents. Furthermore, as exemplified herein, a foam containing a polyol and/or PEG with no water at all can be formed in accordance with the composition and process. Such compositions ensure high stability of water sensitive active agents.
5. Combining the anti-infective effect of a hygroscopic composition, which acts through a dehydration mechanism, with an additional anti-infective agent, selected from the group of an antibiotic agent, an antibacterial agent, an antifungal agent, an agent that controls yeast, an antiviral agent and an antiparasitic agent, that acts through alternate mechanisms results in a synergistic effect and consequently higher success rate of the treatment.
6. The foamable polyol composition in contained in an impermeable pressurized packaging presentation is impermeable and thus, the active agent is not exposed to environmental degradation factors, such as light and oxidizing agent during storage.

Thus, in a preferred embodiment, the composition includes at least one active agent. In the context of herein the at least one active agent is a vitamin and or a flavonoid, a combination of vitamins and or flavonoids, or a combination of at least one vitamin and or at least one flavonoid and at least one additional active agent, as defined herewith.
a. a therapeutically effective concentration of an active agent; and
b. about 50% to about 98% of a polar solvent, selected from the group consisting of a polyol and a polyethylene glycol;
c. 0% to about 48% of a secondary polar solvent;
d. about 0.2% to about 5% by weight of a surface-active agent;
e. about 0.01% to about 5% by weight of at least one polymeric agent; and
f. a liquefied or compressed gas propellant at a concentration of about 3% to about 25% by weight of the total composition.

In the context of combining a hygroscopic carrier according to the present invention, a vitamin and or a flavonoid and an anti-infective active agent, a pharmaceutical composition is provided, including:
a. a vitamin and or a flavonoid;
b. a hygroscopic substance at a sufficient concentration to provide an Aw value of the hygroscopic carrier of less than 0.9. The concentration of the hygroscopic substance in the composition can be designed to provide a Aw value selected from the ranges of (1) about 0.8 and about 0.9; (2) about 0.7 and about 0.8; and (3) less than about 0.7;
c. about 0.2% to about 5% by weight of a surface-active agent;
d. about 0.01% to about 5% by weight of at least one polymeric agent selected from a bioadhesive agent, a gelling agent, a film forming agent and a phase change agent;
e. a therapeutically effective concentration of an anti-infective agent; and
f. a liquefied or compressed gas propellant at a concentration of about 3% to about 25% by weight of the total composition.

In addition to the quasi protective effect of a hygroscopic compound in a formulation to ameliorate, reduce or inhibit the breakdown of a vitamin in the presence of water an exemplary case for the additional inclusion of an anti-infective agent in a hygroscopic composition is provided herewith. It has been surprisingly discovered that combining an antifungal agent in a hygroscopic composition results in an anti-infective effect on strains that are not supposed to be affected by the said antifungal agent. For example, terbinafine is know to be highly effective against dermatophite pathogens, but not against candida. In-vitro studies have revealed, however that terbinafine, dissolved in a hygroscopic carrier, effectively inhibited the spreading of candida albicans, while a control preparation, comprising the same concentration of terbinafine in an emulsion base was not effective. Thus, combining an antifungal agent in a hygroscopic composition results in an expansion of the spectrum of infective strains that can benefit form the therapy; and furthermore, in can render an improved effect of such a composition on mixed infections or in infections that are not accurately diagnosed.

Consequently, in another aspect, a pharmaceutical vitamin and or flavonoid composition, which possesses an improved antifungal activity or that possesses an antifungal activity on an expanded spectrum of pathogens, is provided, including:
a. a hygroscopic composition, comprising a hygroscopic substance at a sufficient concentration to provide an Aw value of the hygroscopic carrier of less than 0.9. The concentration of the hygroscopic substance in the composition can be designed to provide a Aw value selected from the ranges of (1) about 0.8 and about 0.9; (2) about 0.7 and about 0.8; and (3) less than about 0.7;
b. an anti-infective agent, selected from the group of an antibiotic agent, an antibacterial agent, an antifungal agent, an agent that controls yeast, an antiviral agent and an antiparasitic agent. Preferably, the anti-infective agent is an antifungal agent, and more preferably the anti-infective agent is terbinafine.

In another aspect, at least one anti-infective agent, vitamins and or flavonoids are loaded into microsponges and are delivered in a foam formulation to a target.

Active Agents

Vitamins

The term vitamin includes those vitamins and derivatives and analogs thereof (including salts) which are officially recognized as vitamins, and those vitamins which were once recognized or designated as vitamins but are now classified in another way (e.g. vitamin F) and pseudo vitamins including those substances which are a member of a group or complex but are not formally recognized (e.g. para-amino benzoic acid (PABA), which is claimed to prevent greying hair and to be useful as an anti aging supplement) and also vitamin mimetics, which have vitamin like properties or effects.

Suitable vitamins include vitamin A, vitamins of the B complex B1, B2, B3, B5, B6, B7, B9, B12, vitamin C, vitamins D1-D4, vitamin E, vitamin K and so called vitamin F and a derivative thereof and combinations thereof.

Vitamin A is a fat-soluble vitamin and describes compounds that exhibit the biological activity of retinol. The two main components in foods are retinol and the carotenoids. ‘Retinoid’ refers to the chemical entity retinol or other closely related naturally occurring derivatives. These include: retinal (retinaldehyde); retinoic acid; and retinyl esters (e.g. retinyl acetate, retinyl palmitate, retinyl propionate). Retinoids also include structurally related synthetic analogues which may or may not have retinol-like activity. Vitamin A (in the form of retinal) is essential for normal function of the retina and particularly for visual adaptation to darkness. Other forms (retinol, retinoic acid) are necessary for maintenance of the structural and functional integrity of epithelial tissue and the immune system, cellular differentiation and proliferation, bone growth, testicular and ovarian function and embryonic development. It may act also as a co-factor in biochemical reactions. Deficiency can amongst other things result in skin dryness and papular eruptions. Vitamin A and its derivatives have the ability to normalize keratinization. Note that vitamin C may ameliorate the toxic effects of vitamin A; that large doses increase the need for vitamin E; and that vitamin E protects against the oxidative destruction of vitamin A. Retinol is susceptible to breakdown from oxygen and light. Synthetic retinoids may be used for skin problems (e.g. acne).

According to certain embodiments the retinoid is selected from the group consisting of: (1) a compound consisting of four isoprenoid units joined in a head-to-tail manner, a compound having the formula:

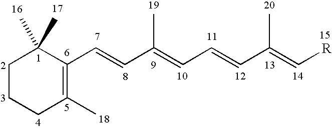

where R is selected from the group consisting of H, alkyl, aryl, alkenyl, benzyl, CH2OH, CH2NH2, CHO, CH═NOH, CO2H, CH═N[CH2]4CHNH2CO2H, CH3, CO2C2H5, CH2OCOCH3, a heteroatom, a saccharide and a polysaccharide; (2) a compound selected from the group consisting of a hydro retinoid, a dehydro retinoid, 3,4-Didehydroretinol, 4,5-Didehydro-5,6-dihydroretinol, a substituted derivative of a retinoid, 5,6-epoxy-5,6-dihydroretinol, ethyl 12-fluororetinoate, a seco retinoid, 1,6-Seco-1,2-didehydroretinol, a nor retinoid, (3) a compound which results from the elimination of a CH3, CH2, CH or C group from a retinoid, N-ethyl-3-methoxy-2-methyl-17-nor-1,2,3,4-tetradehydroretinamide, ethyl 3-methoxy-2-methyl-17-nor-1,2,3,4-tetradehydroretinoate, 5-acetyl-4,18-dinor-retinoic acid, a retro retinoid, 4,5-didehydro-15,5-retro-deoxyretinol, 4,14-retro-retinyl acetate, a stereoisomer of a retinoid, (3R)-3-hydroxyretinol, (3R)-3-Acetoxyretinol, (7E,9E,11E,13Z)-retinoic acid, (6E,8E,10E,12E,15Z)-4,14-retro-retinaloxime, an arotinoids, a retinoidal benzoic acid derivative, 6-[3-(1-adamantyl)-4-methoxyphenyl]-2-naphthoic acid, a short retinoid, a short heterocyclic retinoid, an isoxazole-containing retinoids, a heterocyclic isoxazole-containing retinoid, an isoxazoline-containing retinoid, a stilbene retinoid analog, a retinoid precursor, (ethyl 6-[2-(4,4-dimethylthiochroman-6-yl)ethynyl]nicotinate, a carotene, a xanthophil and an oxicarotenoid; (4) a compound selected from the group consisting of retinol, retinal, retinoic acid, all-trans retinoic acid, isotretinoin, tretinoin, tazarotene, adapalene, 13-cis-retinoic acid, acitretin, all-trans beta carotene, alpha carotene, lycopene, 9-cis-beta-carotene, lutein and zeaxanthin; (5) a compound that is positively identified using a laboratory method, suitable of detecting a retinoid, and salts and derivatives thereof.

Vitamin B is known as the vitamin B complex and comprises B1 (thiamine), B2 (riboflavin), B3 (niacin), B5 (pantothenic acid), B6 (pyridoxine), B7 (biotin), B9 (folic acid) and B12 (cyanocobalamin). Adequate amounts of all B vitamins are required for optimal functioning; deficiency or excess of one B may lead to abnormalities in the metabolism of another.

Thiamine is a water soluble vitamin and is also known as aneurine and functions as a co-enzyme in the oxidative decarboxylation of alpha ketoacids (involved in energy production) and in the transketolase reaction of the pentose phosphate pathway (involved in carbohydrate metabolism). Thiamine is also important in nerve transmission (independently of co-enzyme function). It may also act as an insect repellant.

Riboflavin is a water soluble vitamin and functions as a component of two flavin co-enzymes—flavin mononucleotide (FMN) and flavin adenine dinucleotide (FAD). It participates in oxidation-reduction reactions in numerous metabolic pathways and in energy production. Examples include: the oxidation of glucose, certain amino acids and fatty acids; reactions with several intermediaries of the Krebs cycle; conversion of pyridoxine to its active co-enzyme; and conversion of tryptophan to niacin. Riboflavin has a role as an antioxidant. It may be involved in maintaining the integrity of erythrocytes. Common forms are riboflavin, riboflavin butyrate and flavin adenine dinucleotide.

Niacin is a water-soluble vitamin and describes the compounds that exhibit the biological properties of nicotinamide. It occurs as nicotinamide and nicotinic acid. It is sometimes known as niacinamide. An example of a derivative is benzyl nicotinate. Niacin functions as a component of two co-enzymes, nicotinamide adenine dinucleotide (NAD) and nicotinamide adenine dinucleotide diphosphate (NADP). These co-enzymes participate in many metabolic processes including glycolysis, tissue respiration, lipid, amino acid and purine metabolism. It has been shown to have anti-inflammatory properties that result in the improvement of acne. Topically it has showed benefit for various skin conditions including psoriasis and rosacea. It has also been said to have a photo protection role, perhaps through anti-oxidant activity and reduces or prevents UV damage to cells and UV induced disorders.

Pantothenic acid is also a water soluble vitamin and functions mainly as a component of co-enzyme A and acyl carrier protein. Co-enzyme A has a central role as a co-factor for enzymes involved in the metabolism of lipids, carbohydrates and proteins; it is also required for the synthesis of cholesterol, steroid hormones, acetylcholine and porphyrins. As a component of acyl carrier protein, pantothenic acid is involved in various transfer reactions and in the assembly of acetate units into longer-chain fatty acids. Pantothenic acid has been used for a wide range for disorders such as acne, alopecia, allergies, burning feet, asthma, grey hair, dandruff, and cholesterol lowering. Panthenol the alcoholic form functions as a humetic. Examples of pantothenic acid derivatives are calcium pantothenate, D-pantothenyl alcohol, pantothenyl ethyl ether, and acetylpentothenyl ethyl ether.

Vitamin B6 is water soluble vitamin. Vitamin B6 a generic term used to describe the compounds that exhibit the biological activity of pyridoxine. It occurs in food as pyridoxine, pyridoxal and pyridoxamine. Vitamin B6 is converted in erythrocytes to pyridoxal phosphate and, to a lesser extent, pyridoxamine phosphate. It acts as a co-factor for enzymes which are involved in more than 100 reactions that affect protein, lipid and carbohydrate metabolism. Pyridoxal phosphate is also involved in: the synthesis of several neurotransmitters; the metabolism of several vitamins (e.g. the conversion of tryptophan to niacin); haemoglobin and sphingosine formation. Lack of Vitamin B6 may affect vitamin C. Examples are pyridoxine hydrochloride and pyridoxine dioctanate.

Biotin is a water soluble vitamin which was formerly known as vitamin H or co-enzyme R. Biotin functions as an integral part of the enzymes that transport carboxyl units and fix carbon dioxide. Biotin enzymes are important in carbohydrate and lipid metabolism, and are involved in gluconeogenesis, fatty acid synthesis, propionate metabolism and the catabolism of amino acids. Biotin has been claimed to be of value in the treatment of brittle finger nails, acne, seborrhoeic dermatitis, hair fragility and alopecia.

Folic acid (pteroylglutamic acid) is a water soluble vitamin and is the parent compound for a large number of derivatives collectively known as folates. Folate is the generic term used to describe the compounds that exhibit the biological activity of folic acid; it is the preferred term for the vitamin present in foods which represents a mixture of related compounds (folates). Folates are involved in a number of single carbon transfer reactions, especially in the synthesis of purines and pyrimidines (and hence the synthesis of DNA), glycine and methionine. They are also involved in some amino acid conversions and the formation and utilization of formate. Deficiency leads to impaired cell division (effects most noticeable in rapidly regenerating tissues).

Vitamin B12 is a water-soluble vitamin and it is the generic term used to describe the compounds that exhibit the biological activity of cyanocobalamin. It includes a range of cobalt-containing compounds, known as cobalamins. Cyanocobalamin and hydroxocobalamin are the two principal forms in clinical use. Vitamin B12 is involved in the recycling of folate co-enzymes and the degradation of valine. It is also required for nerve myelination, cell replication, haematopoiesis and nucleoprotein synthesis.

Vitamin C is a water-soluble vitamin and describes the compounds that exhibit the biological activity of ascorbic acid. These include L-ascorbic acid (ascorbic acid) and L-dehydroascorbic acid (dehydroascorbic acid). The functions of vitamin C are based mainly on its properties as a reducing agent. It is required for: the formation of collagen and other organic constituents of the intercellular matrix in bone, teeth and capillaries; and the optimal activity of several enzymes—it activates certain liver-detoxifying enzyme systems (including drug-metabolizing enzymes) and is involved in the synthesis of carnitine and norepinephrine (noradrenaline) and in the metabolism of folic acid, histamine, phenylalanine, tryptophan and tyrosine. Vitamin C also acts: as an antioxidant (reacting directly with aqueous free radicals)—which is important in the protection of cellular function; and to enhance the absorption of non-haem iron. It can function as a whitening agent. Vitamin C may assist with wound healing. Vitamin C can spare vitamin E and vice versa and it may reduce toxic effects of vitamin A. Vitamin C is unstable in solution especially alkaline solution and readily undergoes oxidation on exposure to air. Oxidation is accelerated by light and heat. Cosmetic forms include calcium ascorbate, magnesium ascorbate, sodium ascorbate, sodium ascorbyl phosphate, ascorbyl palmitate, magnesium ascorbyl phosphate, L-ascorbic acid and magnesium-L-ascorbyl-2-phosphate, L-ascorbic acid palmitate, L-ascorbic acid 2-sulfate, L-ascorbic acid phosphate, and DL-.alpha.-tocopherol-L-ascorbic acid phosphate diester dipotassium. L-ascorbic acid is the most bioactive form and has been found to have many skin benefits but it is unstable in the presence of water and oxygen. Inclusion of ascorbic acid in the vitamin carrier, wherein the composition does not contain or is essentially free of water or wherein water is not freely available due to the hygroscopic properties of the composition r and or is not exposed to air during storage makes it possible to derive stable products with the most bioactive form of vitamin C.

Vitamin D is a fat-soluble vitamin and describes all sterols that exhibit the biological activity of cholecalciferol. These include: vitamin D₁ (calciferol), vitamin D₂ (ergocalciferol) vitamin D₃ (cholecalciferol), 1 (OH)D₃ (1 Hydroxycholecalciferol; alfacalcidol), 25(OH)D₃ (25 Hydroxycholecalciferol; calcifediol), 1,25(OH)₂D₃ (1,25, Dihydroxycholecalciferol; calcitriol), 24,25(OH)₂D₃ (24,25, Dihydroxycholecalciferol) and dihydrotachysterol, calcipotriene, 25-hydroxycholecalciferol, 1α,25-dihydroxycholecalciferol, 1α,25-dihydroxyergocalciferol, 22,23-dihydroergocalciferol, 1,24,25-trihydroxycholecalciferol, previtamin D₃, tachysterol₃ (also termed tacalciol), isovitamin D₃, dihydrotachysterol₃, (1S)-hydroxycalciol, (24R)-hydroxycalcidiol, 25-fluorocalciol, ercalcidiol, ertacalciol, (5E)-isocalciol, 22,23-dihydroercalciol, (24S)-methylcalciol, (5E)-(10S)-10,19-dihydroercalciol, (24S)-ethylcalciol and (22E)-(24R)-ethyl-22,23-didehydrocalciol. Vitamin D is essential for promoting the absorption and utilization of calcium and phosphorus, and normal calcification of the skeleton. Along with parathyroid hormone and calcitonin, it regulates serum calcium concentration by altering serum calcium and phosphate blood levels, as needed, and mobilizing calcium from bone. It maintains neuromuscular function and various other cellular processes, including the immune system. Calcipotriene, as well as other vitamin C forms is useful in the treatment of psoriasis.

Vitamin E is a fat-soluble vitamin and describes all tocopherol and tocotrienol derivatives that exhibit the biological activity of alpha tocopherol. Those used commercially are d-alpha tocopherol (natural vitamin E), d-alpha tocopherol acetate, d-alpha tocopherol succinate, d,l-alpha tocopherol (synthetic vitamin E), d,l-alpha tocopherol acetate and d,l-alpha tocopherol succinate. Vitamin E is an antioxidant, protecting polyunsaturated fatty acids in membranes and other critical cellular structures from free radicals and products of oxidation. It works in conjunction with dietary selenium (a co-factor for glutathione peroxidase), and also with vitamin C and other enzymes, including superoxide dismutase and catalase. Vitamin E is not very stable. It may have an anti-inflammatory effect and some studies state that it improves immune function in the elderly. It is also said to reduce oxidative damage and to improve lung function. Vitamin E can spare vitamin C and vice versa. It is said to be photo protective and to have an anti aging effect on skin showing reduced wrinkles and tumors.

Vitamin K is a fat soluble vitamin and describes 2-methyl-1,4-naphthaquinone and all derivatives that exhibit qualitatively the biological activity of phytomenadione. The form of vitamin K present in foods is phytomenadione (vitamin K₁). The substances synthesized by bacteria are known as menaquinones (vitamin K₂). The parent compound of the vitamin K series is known as menadione (vitamin K₃); it is not natural substance and is not used in humans. Menadiol sodium phosphate is water-soluble derivative of menadione. Vitamin K is an essential co-factor for the hepatic synthesis of proteins involved in the regulation of blood clotting. These are: prothrombin (factor II), factors VII, IX, X and proteins C, S and Z. Vitamin K is responsible for the carboxylation of the bone protein, osteocalcin, to its active form. Osteocalcin regulates the function of calcium in bone turnover and mineralization. Vitamin K is also required for the biosynthesis of some other proteins found in plasma and the kidney. It is reported to speed up resolution of bruising to decrease future bruising and correct aspects of photoaging.

Pseudo vitamins: Vitamin F was the designation originally given to essential fatty acids that the body cannot manufacture. They were “de-vitaminized” because they are fatty acids. Fatty acids are a major component of fats which, like water, are needed by the body in large quantities and thus do not fit the definition of vitamins which are needed only in trace amounts. Herbalists and naturopaths have named various therapeutic chemicals “vitamins”, even though they are not, including vitamin T, S-Methylmethionine (vitamin U) and vitamin X. Some authorities say that ubiquinone, also called coenzyme Q10, is a vitamin. Ubiquinone is manufactured in small amounts by the body, like vitamin D. Pangamic acid, vitamin B15; the related substance dimethylglycine is quite wrongly referred to as vitamin B15 but also labeled B16. The toxins laetrile and amygdaline are sometimes referred to as vitamin B17. Both pangamic acid and laetrile were first proposed as vitamins by Ernst T. Krebs; neither are recognized by the medical community. Flavonoids are sometimes called vitamin P. Animal, bird, and bacterial growth factors have been designated vitamins such as para-aminobenzoic acid (PABA) vitamin B₁₀, the folacin (see folic acid) pteryl-heptaglutamic acid vitamin B₁, or vitamin Bc-conjugate and orotic acid as vitamin B₁₃. A few substances were once thought to be B-complex vitamins and are referred to as B-vitamins in older literature, including B₄ (adenine) and B₈ (adenylic acid), but are no longer recognized as such. An antitumor pterin phosphate named Vitamin B-14 and later abandoned.

Vitamins as anti oxidants. The antioxidant vitamins can be divided into those that are water-soluble and exist in aqueous solution—primarily vitamin C—and those that are fat-soluble and exist in membranes or lipoproteins—vitamin E and betacarotene. Lipid membranes are particularly vulnerable to oxidative breakdown by free radicals. Vitamin E protects cell membranes from destruction by undergoing preferential oxidation and destruction. Some quinones, such as ubiquinone (co-enzyme Q) also appear to have antioxidant properties. All these substances can act as free radical scavengers and can react directly with free radicals. Riboflavin also has a role as an antioxidant.

They are believed to protect against certain diseases by preventing the deleterious effects of free-radical-mediated processes in cell membranes and by reducing the susceptibility of tissues to oxidative stress. An article by MP Ludo entitled “Antioxidants and Vitamins in Cosmetics” Clinics in Dermatology (2001): 19: 467-473 discusses the benefits of vitamins and derivatives in cosmetics.

Note that carotenoids and flavonoids also act as antioxidants.

Synergism between vitamins is known, for example, synergism between vitamin A and vitamin E is described by Gallarate, Carlotti, Trotta, and Bovo in the International Journal of Pharmaceutics 188 (1999) 233-241 discussing a study on the stability of ascorbic acid. Any synergism known in the literature between vitamins to potentiate or facilitate their action can be used in the present invention. Details of the solubility of vitamins can be found for example in the Merck Index and other similar reference works and databases.

In one or more embodiments the vitamin is selected from the group consisting of vitamin A, B1, B2, B3, B5, B6, B7, B9, B12, PABA, C, D1-D4, E, K and F and a derivative thereof.

In another embodiment the vitamin is selected from the group consisting of vitamin B1, B2, B3, B5, B6, B7, B9, B12, PABA and C and a derivative thereof.

In an embodiment the vitamin is vitamin B3 or a derivative thereof or combinations thereof.

In an embodiment the vitamin is vitamin C or a derivative thereof or combinations thereof.

In an embodiment the vitamin is the vitamin is vitamin K or a derivative thereof or combinations thereof.

In an embodiment the vitamin is vitamin A or a derivative thereof or combinations thereof.

In an embodiment the vitamin is vitamin E or a derivative thereof or combinations thereof.

In an embodiment the vitamin is the vitamin is vitamin F or a derivative thereof or combinations thereof.

In one or more embodiments the vitamin is a combination of two or more vitamins selected from the group comprising vitamin A, B3, C, K, E, and F and a derivative thereof.

In one or more embodiments the vitamin is a combination of two or more vitamins selected from the group comprising vitamin B3, E and C and a derivative thereof.

Flavonoids

Flavonoids (or bioflavonoids) are a large group of polyphenolic antioxidant compounds, which often occur as glycosides and are ubiquitously present in foods of plant origin. Some flavonoids (e.g. quercetin, rutin) are available as dietary supplements. Flavonoids can be further subdivided into:
flavonols (e.g. kaempferol, quercetin and myricetin)
flavones (e.g. apigenin and luteolin)
flavonones (e.g. hesperetin, naringenin, eriodictyol)
flavan-3-ols (e.g. (+)-catechin, (+)-gallocatechin, (−)-epicatechin, (−)-epigallocatechin)
anthocyanins (e.g. cyanidin, delphinidin, malvidin, pelargonidin, peonidin, petunidin)
proanthocyanidins.

More than 4000 flavonoids have been identified, and many have been studied. Most are colorless but some are responsible for the bright colors of many fruit and vegetables. Flavonoids are distinguished from the carotenoids.

Flavonoids appear to
act as scavengers of free radicals, including superoxide anions, singlet oxygen, and lipid peroxyl radicals (they have antioxidant properties);
sequester metal ions;
inhibit in vitro oxidation of LDL cholesterol;
inhibit cyclo-oxygenase, leading to lower platelet aggregation, decreased thrombotic tendency and reduced anti-inflammatory activity;
inhibit histamine release;
improve capillary function by reducing fragility of capillary walls and thus preventing abnormal leakage; and
inhibit various stages of tumor development (animal studies only).

The activities of flavonoids are dependent on their chemical structure. Estimates of dietary flavonoid intake vary from 10 to 100 mg daily, but may be several hundreds of milligrams a day. Dietary supplements of quercetin and rutin provide around 500 mg in a single dose.

Flavonoids may have a potential role in the prevention of CVD, cancer and cataracts and possibly other diseases, for anti-viral activity, and they may be useful in treating ulcers. include hemorrhoids, allergy, asthma, menopausal symptoms and the prevention of habitual abortion.

Quercetin

As a dietary supplement, quercetin is promoted for prevention and treatment of atherosclerosis and hyperlipidaemia, diabetes, cataracts, hay fever, peptic ulcer, inflammation, prevention of cancer and for treating prostatitis. A preliminary, double-blind, placebo-controlled trial in chronic non-bacterial prostatitis showed that quercetin reduced pain and improved quality of life, but had no effect on voiding dysfunction.

Rutin

As a dietary supplement, rutin is used to reduce capillary permeability and treat symptoms of varicose veins. In combination with bromelain and trypsin, rutin is used to treat osteoarthritis.

A non limiting list of flavonoid compounds is: benzquercin, diosmin, ethoxazorutoside, flavodate, sodium hesperidin, leucocianido, monoxerutin, oxerutin, quercetin, rutoside, rosmarinic acid. The above information was noted from Dietary Supplements, Electronic Version, Pharmaceutical Press 2007.

In an embodiment a single flavonoid is provided and in a further embodiment a combination of two or more flavonoid are provided. In certain embodiments the flavonoids act synergistically. In an embodiment water soluble flavonoids are combined with water insoluble flavonoids. It is known for example that whole polyphenolic extracts have greater antioxidant effect than their known individual components. In an embodiment flavonoids are used in combination with other phenolics. In an embodiment one or more flavonoids are provided in combination with one or more vitamins. In an embodiment flavonoids are provided that are more reactive than the vitamins. In an embodiment the flavonoids act as a conservational agent.

Microsponges

The Microsponges are rigid, porous and spongelike round microscopic particles of cross-linked polymer beads (e.g., polystyrene or copolymers thereof), each defining a substantially noncollapsible pore network. The Microsponges can be loaded with an active ingredient and can provide a controlled time release of the active ingredient to skin or to a mucosal membrane upon application of the formulation. The slow release is intended to reduce irritation by the active. Microsponge® delivery technology was developed by Advanced Polymer Systems.

Microsponges have a size range in between 5 to 300 μm depending upon the degree of smoothness or after feel required for the end formulations and can reduce perceived oiliness.

Wide ranges of uses for microsponges incorporated in foamable compositions are suggested aiming to provide increased efficacy for delivery of active agents topically and in a body cavity with enhanced control, spreadability, safety, stability and improved aesthetic properties. Microspheres can store an active agent until its release is triggered by application to the skin surface such as through rubbing and or higher than-ambient skin temperature. Microsponge do not pass through the skin but collect on the skin surface and slowly release the entrapped agent. The empty spheres are washed away with cleansing.

Microsponges may be incorporated in wide ranges of foam formulations. Non limiting outline examples are listed in the Table below and are exemplified in the prophetic examples in Section C of the Examples:

| | | Preferred compositions |
|---|---|---|
| | Minimal ingredients | with |
| Oil in water type | | |
| foams | | |
| High oil | Oil | Foam adjuvant |
| content | Stabilizing surfactant | Gelling agents |
| | Water | |
| Low oil | Oil | Foam adjuvant |
| content | Stabilizing surfactant | Gelling agents |
| | Water | |
| Oils types | Triglycerides | |
| | Fatty acid esters or ethers | |
| | Silicone | |
| | Mineral | |
| | Specials: Jojoba | |
| | Unsaturated oils | |
| Water in oil | Oil | Foam adjuvant |
| foams | Stabilizing surfactant | Gelling agents |
| | Water | |
| Oils type | | |
| Oily foams | Oils fatty acid or alcohols | Stabilizing surfactants |
| Oils type | Mineral oils | |
| | Vaseline (regular or | |
| | Sofematic) | |
| | Triglycerides | |
| | Silicones | |
| | Fatty acid esters or ethers | |
| | Unsaturated oils | |
| | Specials: Jojoba | |
| Hydrophilic | Hydrophilic solvent | Combination of |
| solvents | Stabilizer (surfactant or | surfactants and |
| | Polymer) | gelling agents |
| Solvents | Polyols such as: Propylene | |
| type | glycol, butylenes glycol, | |
| | hexylene glycol | |
| | Polyethylene glycols (PEG) | |
| | Glycofurol | |
| Special solvents | DMI, Transcutol, Ethanol, | |
| | Glycerin, PPG15 stearyl | |
| | ether, | |
| Liquid Wax | Liquid waxes like jojoba oil, | Solid waxes can |
| | isostearic acid and oleyl | be added |
| | alcohol and other wax like | |
| | liquid fatty acids or fatty | |
| | alcohols which | |

In an embodiment the amount of microsponges may be varied from about 1% to about 25% of the formulation, preferably about 5% to 15%.

In an embodiment any active agent suitable for loading in microsponges may be used, such as benzyl peroxide (BPO), tretinoin, hydroquinone, kotoprofen, retinol, fluconazole, ibuprofen, trolamine and the like

In an embodiment the microsponges are loaded with one or more vitamins or with one or more flavonoids or combinations thereof. In another embodiment the vitamins and or flavonoids are fat soluble. In another embodiment they are water soluble.

In an embodiment the vitamin is a retinoid, preferably a vitamin A, more preferably, retinoic acid or isoretinoic acid.

In an embodiment the vitamin is preferably a vitamin D, a derivative or analogue thereof, more preferably calcipotriol or calcitriol or tacalcitol with or without a corticosteroid such as betmethasone or its esters (eg bmv), flucinonide, hydrocortisone or clobetasol proprionate

In an embodiment, since retinoids and BPO can dry the skin, water soluble humectants, e.g., urea, sodium PCA, alpha-hydroxy acids, glycerin and other polyols may be added.

As can be noted from above and herein different types of active agents may be loaded into the microsponges. Accordingly the foam formulation selected in which to disperse the microsponges should be adapted so that the active agent remains substantially entrapped in the microsponges. In another embodiment the active agent is present both in the foam formulation and in the microsponges so that some of the active agent is available for immediate penetration on application of the foam and that other amounts of active agent are provided by slow or controlled release from the microsponges now sitting on the topical surface.

Where the active agent is oil soluble but not water soluble then formulations with minimal or no oil are preferred where the active agent is primarily to be located in the microsponges. So in an embodiment there is no oil in the formulations, only gels. In another embodiment, there are no significant amounts of true oils that solubilize the active ingredients and extract them from the microsponges.

In an embodiment where the active ingredient is insoluble in water and is entrapped in the microsponges there is provided true oil in water emulsion, where the active ingredient is only exposed to the external water phase and does not access the internal oil phase. This may be achieved in an embodiment by formulating an aqueous oil gel with substantial water content.

In an embodiment the foam formulation comprises a large amount of hydrophobic surfactant or hydrophobic complex emulgators which can form a tight close surfactant layer surrounding the oil droplets sitting in the water phase to separate but still holding the water and oil phases together as an emulsion, thereby preventing or at least substantially reducing any leakage of active agent into the oil phase. In a further embodiment there is provided a gel where the gelling agent is not fat soluble so as to prevent or at least substantially reduce leakage.

The methodology of loading microsponges with active agent and amounts that can be loaded are described in WO 01/85102, which is incorporated herein by way of reference. Where Drug Microsponge X % w/w is provided it refers to the microsponges including the trapped drug and any other ingredients incorporated when loading the microsponges.

Additional Therapeutic Agent

Several conditions involve a combination of etiological factors, some of which is affected by a vitamin; and other etiological factors that require an additional therapeutic modality. For example, psoriasis may be treated by a vitamin D as well as a steroid drug, and therefore combined treatment would be beneficial. Likewise, acne, which involves a microbial infection, excessive keratin production, excessive sebum production and inflammation, can benefit from treatment with a combination of a vitamin A, and an additional therapeutic agent, selected from the group consisting of an anti-inflammatory agent, an antibiotic agent, a sebostatice agent and a keratolytic agent. Hence, in many cases, the inclusion of an additional therapeutic agent in the composition, contributes to the clinical activity of the vitamin.

Suitable additional therapeutic agents include but are not limited to active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, antiyeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, vitamins, vitamin D derivatives, wound healing agents and wart removers. As is known to one skilled in the art, in some instances a specific active agent may have more than one activity, function or effect.

In an embodiment, the additional therapeutic agent is an active herbal extract. Suitable active herbal extracts include but are not limited to angelica, anise oil, astragali radix, azalea, benzyl acetate, birch tar oil, bornyl acetate, cacumen biotae, camphor, cantharidin, capsicum, cineole, cinnamon bark, cinnamon leaf, citronella, citroneliol, citronellyl acetate, citronellyl formate, eucalyptus, eugenyl acetate, flos carthami, fructus mori, garlic, geraniol, geranium, geranyl acetate, habanera, isobutyl angelicate, lavender, ledum latifolium, ledum palustre, lemongrass, limonene, linalool, linalyl acetate, methyl anthranilate, methyl cinnamate, mezereum, neem, nerol, neryl acetate, nettle root extract, oleum ricini, oregano, pinenes, .alpha.-pinene, .beta.-pinene, radix angelicae sinesis, radix paenoiae rubra, radix polygoni multiflori, radix rehmanniae, rhizoma pinelliae, rhizoma zingiberis recens, sabadilla, sage, sandalwood oil, saw palmetto extract, semen sesami nigrum, staphysagria, tea tree oil, terpene alcohols, terpene hydrocarbons, terpene esters, terpinene, terpineol, terpinyl acetate and derivatives, esters, salts and mixtures thereof. In an embodiment, the active agent is an acaricide. Suitable acaricides include but are not limited to amitraz, flumethrin, fluvalinate and derivatives, esters, salts and mixtures thereof.

In an embodiment, the additional therapeutic agent is an age spot and keratoses removing agent. Suitable age spot and keratoses removing agent include but are not limited to hydroxy acids, azelaic acid and other related dicarboxylic acids, retinoids, kojic acid, arbutin, nicotinic, ascorbic acid, hydroquinone and derivatives, esters, salts and mixtures thereof. Certain nonsteroidal anti-inflammatory agents, such as diclofenac are also useful for the treatment of keratoses.

In an embodiment, the additional therapeutic agent is an analgesic. Suitable analgesics include but are not limited to benzocaine, butamben picrate, dibucaine, dimethisoquin, dyclonine, lidocaine, pramoxine, tetracaine, salicylates and derivatives, esters, salts and mixtures thereof.

In an embodiment, the additional therapeutic agent is a local anesthetic. Suitable local anesthetics include but are not limited to benzocaine, benzyl alcohol, bupivacaine, butamben picrate, chlorprocaine, cocaine, dibucaine, dimethisoquin, dyclonine, etidocaine, hexylcaine, ketamine, lidocaine, mepivacaine, phenol, pramoxine, procaine, tetracaine, salicylates and derivatives, esters, salts and mixtures thereof.

In an embodiment, the additional therapeutic agent is an antiacne agent. Suitable antiacne agents include but are not limited to N-acetylcysteine, adapalene, azelaic acid, benzoyl peroxide, cholate, clindamycin, deoxycholate, erythromycin, flavonoids, glycolic acid, meclocycline, metronidazol, mupirocin, octopirox, phenoxy ethanol, phenoxy proponol, pyruvic acid, resorcinol, retinoic acid, salicylic acid, scymnol sulfate, sulfacetamide-sulfur, sulfur, tazarotene, tetracycline, tretinoin triclosan and derivatives, esters, salts and mixtures thereof.

In an embodiment, the additional therapeutic agent is an antiaging agent. Suitable antiaging agents include but are not limited to sulfur-containing D and L amino acids, alpha-hydroxy acids s, beta-hydroxy acids (e.g. salicylic acid), urea, hyaluronic acid, phytic acid, lipoic acid; lysophosphatidic acid, skin peel agents (e.g., phenol, resorcinol and the like), vitamin B3 compounds (e.g., niacinamide, nicotinic acid and nicotinic acid salts and esters, including non-vasodilating esters of nicotinic acid (such as tocopheryl nicotinate), nicotinyl amino acids, nicotinyl alcohol esters of carboxylic acids, nicotinic acid N-oxide and niacinamide N-oxide), vitamin B5 and retinoids (e.g., retinol, retinal, retinoic acid, retinyl acetate, retinyl palmitate, retinyl ascorbate) skin barrier forming agents, melatonin and derivatives, esters, salts and mixtures thereof.

In an embodiment, the additional therapeutic agent is an antibiotic. The terms “antibiotic” as used herein shall include, but is not limited to, any substance being destructive to or inhibiting the growth of bacteria or any substance having the capacity to inhibit the growth of or to destroy bacteria. In one or more embodiments, the antibiotic agent is selected from the group consisting of a beta-lactam antibiotic, an aminoglycoside, an ansa-type antibiotic, an anthraquinone, an azole, an antibiotic glycopeptide, a macrolide, an antibiotic nucleoside, an antibiotic peptide, an antibiotic polyene, an antibiotic polyether, an antibiotic quinolone, an antibiotic steroid, a sulfonamide, an antibiotic metal, an oxidizing agent, a periodate, a hypochlorite, a permanganate, a substance that release free radicals and/or active oxygen, a cationic antimicrobial agent, a quaternary ammonium compound, a biguanide, a triguanide, a bisbiguanide, a polymeric biguanide, and analogs, derivatives, salts, ions and complexes thereof.

Suitable antibiotics include but are not limited to amanfadine hydrochloride, amanfadine sulfate, amikacin, arnikacin sulfate, aminoglycosides, amoxicillin, ampicillin, ansamycins, bacitracin, beta-lactams, candicidin, capreomycin, carbenicillin, cephalexin, cephaloridine, cephalothin, cefazolin, cephapirin, cephradine, cephaloglycin, chloramphenicols, chlorhexidine, chlorhexidine gluconate, chlorhexidine hydrochloride, chloroxine, chlorquinaldol, chlortetracycline, chlortetracycline hydrochloride, ciprofloxacin, circulin, clindamycin, clindamycin hydrochloride, clotrimazole, cloxacillin, demeclocycline, diclosxacillin, diiodohydroxyquin, doxycycline, ethambutol, ethambutol hydrochloride, erythromycin, erythromycin estolate, erythromycin stearate, farnesol, floxacillin, gentamicin, gentamicin sulfate, gramicidin, griseofulvin, haloprogin, haloquinol, hexachlorophene, iminocyldline, iodate, iodine, iodochlorhydroxyquin, kanamycin, kanamycin sulfate, lincomycin, lineomycin, lineomycin hydrochloride, macrolides, meclocycline, methacycline, methacycline hydrochloride, methenamine, methenamine hippurate, methenamine mandelate, methicillin, metronidazole, miconazole, miconazole hydrochloride, microcrystalline and nanocrystalline particles of silver, copper, zinc, mercury, tin, lead, bismuth, cadmium and chromium, minocycline, minocycline hydrochloride, mupirocin, nafcillin, neomycin, neomycin sulfate, netilmicin, netilmicin sulfate, nitrofurazone, norfloxacin, nystatin, octopirox, oleandomycin, orcephalosporins, oxacillin, oxytetracycline, oxytetracycline hydrochloride, parachlorometa xylenol, paromomycin, paromomycin sulfate, penicillins, penicillin G, penicillin V, pentamidine, pentamidine hydrochloride, phenethicillin, polymyxins, quinolones, streptomycin sulfate, tetracycline, tobramycin, tolnaftate, triclosan, trifampin, rifamycin, rolitetracycline, spectinomycin, spiramycin, streptomycin, sulfonamide, tetracyclines, tetracycline, tobramycin, tobramycin sulfate, triclocarbon, triclosan, trimethoprim-sulfamethoxazole, tylosin, vancomycin, yrothricin and derivatives, esters, salts and mixtures thereof.

In an embodiment, the additional therapeutic agent is an antidandruff agent. Suitable antidandruff agents include but are not limited to aminexil, benzalkonium chloride, benzethonium chloride, 3-bromo-1-chloro-5,5-dimethyl-hydantoin, chloramine B, chloramine T, chlorhexidine, N-chlorosuccinimide, climbazole-, 1,3-dibromo-5,5-dimethylhydantoin, 1,3-dichloro-5,5-dimethyl-hydantoin, betulinic acid, betulonic acid, celastrol, crataegolic acid, cromakalin, cyproterone acetate, dutasteride, finesteride, ibuprofen, ketoconozole, oleanolic acid, phenyloin, picrotone olamine, salicylic acid, selenium sulphides, triclosan, triiodothyronine, ursolic acid, zinc gluconate, zinc omadine, zinc pyrithione and derivatives, esters, salts and mixtures thereof.

In an embodiment, the additional therapeutic agent is an antihistamine. Suitable antihistamines include but are not limited to chlorcyclizine, diphenhydramine, mepyramine, methapyrilene, tripelennamine and derivatives, esters, salts and mixtures thereof.

In an embodiment, the additional therapeutic agent is an antimycotic Also termed antifungal agent. The terms “antimycotic” and “antifungal” as used herein include, but is not limited to, any substance being destructive to or inhibiting the growth of fungi and yeast or any substance having the capacity to inhibit the growth of or to destroy fungi and/or yeast.

In one or more embodiments, the antifungal agent is an agent that is useful in the treatment of a superficial fungal infection of the skin, dermatophytosis, microsporum, trichophyton and epidermophyton infections, candidiasis, oral candidiasis (thrush), candidiasis of the skin and genital mucous membrane, candida paronychia, which inflicts the nail and nail bed and genital and vaginal candida, which inflict genitalia and the vagina.

Suitable antimycotics include but are not limited to allylamines, amorolfine, amphotericin B, azole compounds, bifonazole, butoconazole, chloroxine, clotrimazole, ciclopirox olamine, clotrimazole, econazole, elubiol, fenticonazole, fluconazole, flucytosine (5FC), griseofulvin, itraconazole, ketoconazole, mafenide acetate, miconazole, naftifine, natamycin, tolnaftate, nystatin, polyenes, oxiconazole, sulbentine, sulconazole, terbinafine, terconazole, tioconazole, undecylenic acid and derivatives, esters, salts and mixtures thereof.

In an embodiment, the active agent is an antipruritic. Suitable antipruritics include but are not limited to menthol, methdilazine, trimeprazine, urea and derivatives, esters, salts and mixtures thereof.

In an embodiment, the additional therapeutic agent is an additional antipsoriatic agent. Suitable additional antipsoriatic agents include but are not limited to 6-aminonicotinamide, 6-aminonicotinic acid, 2-aminopyrazinamide, anthralin, 6-carbamoylnicotinamide, 6-chloronicotinamide, 2-carbamoylpyrazinamide, corticosteroids, 6-dimethylaminonicotinamide, dithranol, 6-formylaminonicotinamide, 6-hydroxy nicotinic acid, 6-substituted nicotinamides, 6-substituted nicotinic acid, 2-substituted pyrazinamide, tazarotene, thionicotinamide, trichothecene mycotoxins and derivatives, esters, salts and mixtures thereof.

In an embodiment, the active agent is an antirosacea agent. Suitable antirosacea agents include but are not limited to azelaic acid, metronidazole, sulfacetamide and derivatives, esters, salts and mixtures thereof. Certain nonsteroidal anti-inflammatory agents, such as salicylic acid, salycilates, piroxicam and diclofenac are also useful for the treatment of Rosacea.

In an embodiment, the additional therapeutic agent is an antiseborrheic agent. Suitable antiseborrheic agents include but are not limited to glycolic acid, salicylic acid, selenium sulfide, zinc pyrithione, a dicarboxylic acid, such as azelaic acid and derivatives, esters, salts and mixtures thereof.

In an embodiment, the additional therapeutic agent is an antiviral agent. Suitable antiviral agents include but are not limited to acyclovir, gancyclovir, ribavirin, amantadine, rimantadine nucleoside-analog reverse transcriptase inhibitors, such as zidovudine, didanosine, zalcitabine, tavudine, lamivudine and vidarabine, non-nucleoside reverse transcriptase inhibitors, such as nevirapine and delavirdine, protease inhibitors, such as saquinavir, ritonavir, indinavir and nelfinavir, and interferons and derivatives, esters, salts and mixtures thereof.

In an embodiment, the additional therapeutic agent is a chemotherapeutic agent. Suitable chemotherapeutic agents include but are not limited to daunorubicin, doxorubicin, idarubicin, amrubicin, pirarubicin, epirubicin, mitoxantrone, etoposide, teniposide, vinblastine, vincristine, mitomycin C, 5-FU, paclitaxel, docetaxel, actinomycin D, colchicine, topotecan, irinotecan, gemcitabine cyclosporin, verapamil, valspodor, probenecid, MK571, GF120918, LY335979, biricodar, terfenadine, quinidine, pervilleine A, XR9576 and derivatives, esters, salts and mixtures thereof.

In an embodiment, the additional therapeutic agent is a corticosteroid. Suitable corticosteroids include but are not limited to alclometasone dipropionate, amcinafel, amcinafide, amcinonide, beclomethasone, beclomethasone dipropionate, betamethsone, betamethasone benzoate, betamethasone dexamethasone-phosphate, dipropionate, betamethasone valerate, budesonide, chloroprednisone, chlorprednisone acetate, clescinolone, clobetasol, clobetasol propionate, clobetasol valerate, clobetasone, clobetasone butyrate, clocortelone, cortisone, cortodoxone, craposone butyrate, desonide, desoxymethasone, dexamethasone, desoxycorticosterone acetate, dichlorisone, diflorasone diacetate, diflucortolone valerate, difluorosone diacetate, diflurprednate, fluadrenolone, flucetonide, flucloronide, fluclorolone acetonide, flucortine butylesters, fludroxycortide, fludrocortisone, flumethasone, flumethasone pivalate, flumethasone pivalate, flunisolide, fluocinolone, fluocinolone acetonide, fluocinonide, fluocortin butyl, fluocortolone, fluorometholone, fluosinolone acetonide, fluperolone, fluprednidene acetate, fluprednisolone hydrocortamate, fluradrenolone, fluradrenolone acetonide, flurandrenolone, fluticasone, halcinonide, halobetasol, hydrocortisone, hydrocortisone acetate, hydrocortisone butyrate, hydrocortisone cyclopentylpropionate, hydrocortisone valerate, hydroxyltriamcinolone, medrysone, meprednisone, .alpha.-methyl dexamethasone, methylprednisolone, methylprednisolone acetate, mometasone furoate, paramethasone, prednisolone, prednisone, pregnenolone, progesterone, spironolactone, triamcinolone, triamcinolone acetonide and derivatives, esters, salts and mixtures thereof.

In an embodiment, the active agent is a hair growth regulator. Suitable hair growth regulators include but are not limited to N-acetylgalactosamine, N-acetylglucosamine, N-acetylmannosamine, acitretin, aminexil, ascomycin, asiatic acid, azelaic acid, benzalkonium chloride, benzethonium chloride, benzydamine, benzyl nicotinate, benzoyl peroxide, benzyl peroxide, betulinic acid, betulonic acid, calcium pantothenate, celastrol, cepharanthine, chlorpheniramine maleate, clinacycin hydrochloride, crataegolic acid, cromakalin, cyproterone acetate, diazoxide, diphenhydramine hydrochloride, dutasteride, estradiol, ethyl-2-hydroxypropanoate, finasteride, D-fucono-1,5-lactone, furoate, L-galactono-1,4-lactone, D-galactosamine, D-glucaro-1,4-lactone, D-glucosamine-3-sulphate, hinokitiol, hydrocortisone, 2-hydroxypropionic acid, isotretinoin, itraconazole, ketoconazole, latanoprost, 2-methyl propan-2-ol, minocyclin, minoxidil, mipirocin, mometasone, oleanolic acid, panthenol, 1,10-phenanthroline, phenyloin, prednisolone, progesterone, propan-2-ol, pseudoterins, resorcinol, selenium sulfide, tazarotene, triclocarbon, triclosan, triiodothyronine, ursolic acid, zinc pyrithione and derivatives, esters, salts and mixtures thereof.

In an embodiment, the additional therapeutic agent is a hormone. Suitable hormones include but are not limited to methyltestosterone, androsterone, androsterone acetate, androsterone propionate, androsterone benzoate, androsteronediol, androsteronediol-3-acetate, androsteronediol-17-acetate, androsteronediol 3-17-diacetate, androsteronediol-17-benzoate, androsteronedione, androstenedione, androstenediol, dehydroepiandrosterone, sodium dehydroepiandrosterone sulfate, dromostanolone, dromostanolone propionate, ethylestrenol, fluoxymesterone, nandrolone phenpropionate, nandrolone decanoate, nandrolone furylpropionate, nandrolone cyclohexane-propionate, nandrolone benzoate, nandrolone cyclohexanecarboxylate, and rosteronediol-3-acetate-1-7-benzoate, oxandrolone, oxymetholone, stanozolol, testosterone, testosterone decanoate, 4-dihydrotestosterone, 5a-dihydrotestosterone, testolactone, 17a-methyl-19-nortestosterone, desogestrel, dydrogesterone, ethynodiol diacetate, medroxyprogesterone, levonorgestrel, medroxyprogesterone acetate, hydroxyprogesterone caproate, norethindrone, norethindrone acetate, norethynodrel, allylestrenol, 19-nortestosterone, lynoestrenol, quingestanol acetate, medrogestone, norgestrienone, dimethisterone, ethisterone, cyproterone acetate, chlormadinone acetate, megestrol acetate, norgestimate, norgestrel, desogrestrel, trimegestone, gestodene, nomegestrol acetate, progesterone, 5a-pregnan-3b,20a-diol sulfate, 5a-pregnan-3b,20b-diol sulfate, 5a-pregnan-3b-ol-20-one, 16,5a-pregnen-3b-ol-20-one, 4-pregnen-20b-ol-3-one-20-sulfate, acetoxypregnenolone, anagestone acetate, cyproterone, dihydrogesterone, fluorogestone acetate, gestadene, hydroxyprogesterone acetate, hydroxymethylprogesterone, hydroxymethyl progesterone acetate, 3-ketodesogestrel, megestrol, melengestrol acetate, norethisterone, progestins and derivatives, esters, salts and mixtures thereof.

In an embodiment, the additional therapeutic agent is a hydroxyacid. Suitable hydroxy acids include but are not limited to agaricic acid, aleuritic acid, allaric acid, altraric acid, arabiraric acid, ascorbic acid, atrolactic acid, benzilic acid, citramalic acid, citric acid, dihydroxytartaric acid, erythraric acid, galactaric acid, galacturonic acid, glucaric acid, glucuronic acid, glyceric acid, glycolic acid, gularic acid, gulonic acid, hydroxypyruvic acid, idaric acid, isocitric acid, lactic acid, lyxaric acid, malic acid, mandelic acid, mannaric acid, methyllacetic acid, mucic acid, phenyllacetic acid, pyruvic acid, quinic acid, ribaric acid, ribonic acid, saccharic acid, talaric acid, tartaric acid, tartronic acid, threaric acid, tropic acid, uronic acids, xylaric acid and derivatives, esters, salts and mixtures thereof.

In an embodiment, the active agent is a keratolytic agent. The term “keratolytic agent” is used herein to mean a compound which loosens and removes the stratum corneum of the skin, or alters the structure of the keratin layers of skin. Keratolytic agents are used in the treatment of many dermatological disorders, which involve dry skin, hyperkeratiinization (such as prsoriasis), skin itching (such as xerosis), acne and rosacea. Suitable keratolytic agents include but are not limited to N-acetylcysteine, azelaic acid, cresols, dihydroxy benzene compounds, such as resorcinol and hydroquinone, alpha-hydroxy acids, such as lactic acid and glycolic acid, phenol, pyruvic acid, resorcinol, sulfur, salicylic acid, retinoic acid, isoretinoic acid, retinol, retinal, urea and derivatives, esters, salts and mixtures thereof.

In an embodiment, the active agent is a lactam. Suitable lactams include but are not limited to L-galactono-1,4-lactam, L-arabino-1,5-lactam, D-fucono-1,5-lactam, D-glucaro-1,4-lactam, D-glucurono-6,3-lactam, 2,5-tri-O-acetyl-D-glucurono-6,3-lactam, 2-acetamido-2-deoxyglucono-1,5-lactam, 2-acetamido-2-deoxygalactono-1,5-lactam, D-glucaro-1,4:6,3-dilactam-, L-idaro-1,5-lactam, 2,3,5,tri-O-acetyl-D-glucaro-1,4-lactam, 2,5-di-O-acetyl-D-glucaro-1,4:6,3-dilactam, D-glucaro-1,5-lactam methyl ester, 2-propionoamide-2-deoxyglucaro-1,5-lactam and derivatives, esters, salts and mixtures thereof.

In an embodiment, the additional therapeutic agent is a non-steroidal anti-inflammatory agent. Suitable non-steroidal anti-inflammatory agent include but are not limited to azelaic acid, oxicams, piroxicam, isoxicam, tenoxicam, sudoxicam, CP-14,304, salicylates, aspirin, disalcid, benorylate, trilisate, safapryn, solprin, diflunisal, fendosal, acetic acid derivatives, diclofenac, fenclofenac, indomethacin, sulindac, tolmetin, isoxepac, furofenac, tiopinac, zidometacin, acematacin, fentiazac, zomepirac, clindanac, oxepinac, felbinac, ketorolac, fenamates, mefenamic, meclofenamic, flufenamic, niflumic, tolfenamic acids, propionic acid derivatives, ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, tiaprofen, pyrazoles, phenylbutazone, oxyphenbutazone, feprazone, azapropazone, trimethazone and derivatives, esters, salts and mixtures thereof.

In an embodiment, the additional therapeutic agent is insecticide. The term “insecticide, is used herein to mean a compound which kills, inhibits the growth of, impeded the proliferation of or repels insects. Insecticides include, for example, agents that can kill lice, flees, ticks, mites, scabies and mousquitos, as well as agents that repel such insects. Suitable insecticides include but are not limited to DDT, lindane, malathion, permethrin, allethrin, biopermethrin, transpermethrin, phenothrin, diethyl-m-toluamide, dimethyl phthalate, piperonyl butoxide, pyrethroids and derivatives, esters, salts and mixtures thereof.

In an embodiment, the additional therapeutic agent is a vasodilator. Suitable vasodilators include but are not limited to agents that modulate the activity of the enzyme nitric oxide synthase, nicotinic acid, ethyl nicotinate, amyl nitrite, amyl nitrate, ethyl nitrite, butyl nitrite, isobutyl nitrite, glyceryl trinitrate, octyl nitrite, sodium nitrite, sodium nitroprusside, clonitrate, erythrityl tetranitrate, isosorbide mononitrate, isosorbide dinitrate, mannitol hexanitrate, pentaerythritol tetranitrate, penetrinitol, triethanolamine trinitrate, trolnitrate phosphate (triethanolamine trinitrate diphosphate), propatyinitrate, nitrite esters of sugars, nitrite esters of polyols, nitrate esters of sugars, nitrate esters of polyols, nicorandil, apresoline, diazoxide, hydralazine, hydrochlorothiazide, minoxidil, pentaerythritol, tolazoline, scoparone, a beta-adrenergic blocker, an alpha-adrenoceptor blocker, a prostaglandin, sildenafil, dipyridamole, catecholamine, isoproternol, furosemide, prostaglandin, prostacyclin, enalaprilat, morphine, acepromazine, prazosin (α-blocker), enalapril, Captopril, amlodipine, minoxidil, tadalafil, vardenafil, phenylephrin, etilefein, caffeine, capsaicin, an extract capsicum, achillea millefolium (Yarrow), allium sativum (garlic), amoracia rusticana (horseradish), berberis vulgaris (barberry), cimicifuga racemosa (black cohosh), coleus forskholii (coleus), coptis (goldenthread), crataegus (hawthorn), eleutherococcus senticosus (siberian ginseng), ginkgo biloba (ginkgo), melissa offiicnalis (lemon balm), olea europaea (olive leaf), panax ginseng (Chinese ginseng), petroselinum crispum (parsley), scutellaria baicalensis (baical skullcap), tilia europaea (linden flower), trigonella foenum-graecum (fenugreek), urtica dioica (nettles), valeriana officinalis (valerian), viburnum (cramp, bark, black haw), veratrum viride (American hellebore), verbena officinalis (vervain), xanthoxylum americanum (prickly ash), zingiber officinale (ginger), rauwolfia serpentina (Indian snakeroot), viscum album, wild yam, sasparilla, licorice, damiana, yucca, saw palmetto, gotu kola (centella asiatica), yohimbine and salts, hazel nut, brazil nut and walnut, and derivatives, esters, salts and mixtures thereof.

In an embodiment, the additional therapeutic agent is a vasoconstrictor. Suitable vasodilators include but are not limited to ephedrine, epinephrine, phenylephrine, angiotensin, vasopressin; an extract ephedra sinica (ma huang), polygonum bistorta (bistort root), hamamelis virginiana (witch hazel), hydrastis canadensis (goldenseal), lycopus virginicus (bugleweed), aspidosperma quebracho (quebracho blanco), cytisus scoparius (scotch broom) and cypressand and derivatives, esters, salts and mixtures thereof.

In an embodiment, the active agent is a retinoid. Suitable retinoids include but are not limited to retinol, retinal, retinoic acid, all-trans retinoic acid, isotretinoin, tazarotene, adapalene, 13-cis-retinoic acid, acitretin all-trans beta carotene, alpha carotene, lycopene, 9-cis-beta-carotene, lutein and zeaxanthin.

In an embodiment, the additional therapeutic agent is selected from the group consisting of an immunosuppressants and immunoregulating agents. Suitable immunosuppressants and immunoregulating agents include but are not limited to cyclic peptides, such as cyclosporine, tacrolimus, tresperimus, pimecrolimus, sirolimus (rapamycin), verolimus, laflunimus, laquinimod, imiquimod derivatives, esters, salts and mixtures thereof. In one or more embodiments, the immunomodulator is a calcineurin Inhibitor.

In an embodiment, the additional therapeutic agent is a wart remover. Suitable wart removers include but are not limited to imiquimod, podophyllotoxin and derivatives, esters, salts and mixtures thereof.

In an embodiment, the additional therapeutic agent is a photodynamic therapy (PDT) agent. Suitable PDT agents include but are not limited to modified porphyrins, chlorins, bacteriochlorins, phthalocyanines, naphthalocyanines, pheophorbides, purpurins, m-THPC, mono-L-aspartyl chlorine6, bacteriochlorins, phthalocyanines, benzoporphyrin derivatives, as well as photosensitiser precursors, such as aminolevulinic acid and derivatives, esters, salts and mixtures thereof.

In an embodiment, the additional therapeutic agent is an antioxidant or a radical scavenger. Suitable antioxidants and radical scavengers agents include but are not limited to ascorbic acid, ascorbyl esters of fatty acids, magnesium ascorbyl phosphate, sodium ascorbyl phosphate, ascorbyl sorbate, tocopherol, tocopheryl sorbate, tocopheryl acetate, butylated hydroxy benzoic acid, 6-hydroxy-2,5,7,8-tetramethylchroman-2-carboxylic acid, gallic acid, propyl gallate, uric acid, sorbic acid, lipoic acid, diethylhydroxylamine, amino-guanidine, glutathione, dihydroxy fumaric acid, lycine pidolate, arginine pilolate, nordihydroguaiaretic acid, bioflavonoids, curcumin, lysine, methionine, proline, superoxide dismutase, silymarin, tea extracts, grape skin/seed extracts, melanin, and polyunsaturated oils, containing omega-3 and omega-6 fatty acids (e.g., linoleic and linolenic acid, gamma-linoleic acid, eicosapentaenoic acid and docosahexaenoic acid and derivatives, esters, salts and mixtures thereof.

In an embodiment, the additional therapeutic agent is a self-tanning agent, such as dihydroxyacetone.

In an embodiment, the additional therapeutic agent is an agent, capable of treating hyperhidrosis. Suitable hyperhidrosis agents include but are not limited to anticholinergic drugs, boric acid, tannic acid, resorcinol, potassium permanganate, formaldehyde, glutaraldehyde, methenamine, a Lewis acid, aluminum chloride, aluminum chlorohydrates, zirconium chlorohydrates, aluminum-zirconium-Glycine (AZG) complex, aluminum hydroxybromide, a glycopyrrolate compound, a 5-alpha-reductase inhibitor, finasteride, epristeride, flutamide, spironolactone, saw palmetto extract, cholestan-3-one, a mono- and dicarboxylic acid having 4 to 18 carbon atoms, botulinum toxin, a 5-HT2C receptor antagonist, a 5-HT2C receptor antagonist, ketanserin, ritanserin, mianserin, mesulergine, cyproheptadine, fluoxetine, mirtazapine, olanzapine and ziprasidone.

In an embodiment, the additional therapeutic agent is a sunscreen agent. Suitable sunscreen agents include but are not limited to titanium dioxide, zinc oxide, zirconium oxide, iron oxide, p-aminobenzoic acid and its derivatives (ethyl, isobutyl, glyceryl esters; p-dimethylaminobenzoic acid); anthranilic acid derivatives (i.e., o-amino-benzoates, methyl, menthyl, phenyl, benzyl, phenylethyl, linalyl, terpinyl, and cyclohexenyl esters); salicylates (amyl, phenyl, octyl, benzyl, menthyl, glyceryl, and di-pro-pyleneglycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzalacetone and benzalacetophenone; naphtholsulfonates (sodium salts of 2-naphthol-3,6-disulfonic and of 2-naphthol-6,8-disulfonic acids); di-hydroxynaphthoic acid, o- and p-hydroxybiphenyldisulfonates, coumarin derivatives (7-hydroxy, 7-methyl, 3-phenyl), diazoles (2-acetyl-3-bromoindazole, phenyl benzoxazole, methyl naphthoxazole, quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl)ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene bornan-2-one), terephthalylidene dicamphor sulfonic acid and 4-isopropyl-di-benzoylmethane.

In an embodiment, the additional therapeutic agent is a figure-forming agent and an agent, capable of treating cellulite. Suitable such agents include but are not limited to baldderwack extract, butcher's, broom, cayenne, dandelion, red clover, ginkgo biloba, horse chestnut, witch hazel and borage oil, caffeic acid, nicotinic acid, theophiline and pentoxyphilline and salts and derivatives thereof.

Several disorders of the skin, body cavity or mucosal surface (e.g., the mucosa or the cavity of the nose, mouth, eye, ear, vagina or rectum) involve a combination of etiological factors. For example, fungal and bacterial infections and that are inflamed and have symptoms of redness and/or itching warrant therapy that combines an anti-infective agent and an anti-inflammatory agent. Thus, in several cases, combining at least two active agents that treat different etiological factors results in a synergistic effect and consequently higher success rate of the treatment.

In certain cases, the composition contains two active agents, where each of the active agents require a different pH environment in order to remain stable. For example, corticosteroids are typically stable at acidic pH values (they have a maximum stability at a pH of about 4-6) and of vitamin D analogues are typically stable at basic pH values (they have a maximum stability at pH values above about 8). In order to circumvent the problem of instability it is preferred that the composition is substantially non-aqueous. The term “substantially non-aqueous” is intended to indicate that the composition has a water content below about 5%, preferably below about 2%, such as below about 1.5%.

Fields of Applications

The foamable carrier is suitable for treating any infected surface. In one or more embodiments, foamable carrier is suitable for administration to the skin, a body surface, a body cavity or mucosal surface, e.g., the cavity and/or the mucosa of the nose, mouth, eye, ear, respiratory system, vagina or rectum (severally and interchangeably termed herein “target site”).

By selecting a suitable active agent, or a combination of at least two active agents, the foamable composition is useful in treating an animal or a human patient having any one of a variety of dermatological disorders, including dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, ecthyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo.

Likewise, the foamable composition is suitable for treating a disorder of a body cavity or mucosal surface, e.g., the mucosa of the nose, mouth, eye, ear, respiratory system, vagina or rectum. Non limiting examples of such conditions include chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

In an embodiment, the composition is useful for the treatment of an infection. In one or more embodiments, the composition is suitable for the treatment of an infection, selected from the group of a bacterial infection, a fungal infection, a yeast infection, a viral infection and a parasitic infection.

In an embodiment, the composition is useful for the treatment of wound, ulcer and burn. This use is particularly important since the composition creates a thin, semi-occlusive layer, which coats the damaged tissue, while allowing exudates to be released from the tissue.

The composition is also suitable for administering a hormone to the skin or to a mucosal membrane or to a body cavity, in order to deliver the hormone into the tissue of the target organ, in any disorder that responds to treatment with a hormone.

In light of the hygroscopic nature of the composition, it is further suitable for the treatment and prevention of post-surgical adhesions. Adhesions are scars that form abnormal connections between tissue surfaces. Post-surgical adhesion formation is a natural consequence of surgery, resulting when tissue. repairs itself following incision, cauterization, suturing, or other means of trauma. When comprising appropriate protective agents, the foam is suitable for the treatment or prevention of post surgical adhesions. The use of foam is particularly advantageous because foam can expand in the body cavity and penetrate into hidden areas that cannot be reached by any other alternative means of administration.

Other foamable compositions are described in: U.S. Publication No. 05-0232869, published on Oct. 20, 2005, entitled NONSTEROIDAL IMMUNOMODULATING KIT AND COMPOSITION AND USES THEREOF; U.S. Publication No. 05-0205086, published on Sep. 22, 2005, entitled RETINOID IMMUNOMODULATING KIT AND COMPOSITION AND USES THEREOF; U.S. Publication No. 06-0018937, published on Jan. 26, 2006, entitled STEROID KIT AND FOAMABLE COMPOSITION AND USES THEREOF; U.S. Publication No. 05-0271596, published on Dec. 8, 2005, entitled VASOACTIVE KIT AND COMPOSITION AND USES THEREOF; U.S. Publication No. 06-0269485, published on Nov. 30, 2006, entitled ANTIBIOTIC KIT AND COMPOSITION AND USES THEREOF; U.S. Publication No. 07-0020304, published on Jan. 25, 2007, entitled NON-FLAMMABLE INSECTICIDE COMPOSITION AND USES THEREOF; U.S. Publication No. 06-0193789, published on Aug. 31, 2006, entitled FILM FORMING FOAMABLE COMPOSITION; U.S. patent application Ser. No. 11/732,547, filed on Apr. 4, 2007, entitled ANTI-INFECTION AUGMENTATION OF FOAMABLE COMPOSITIONS AND KIT AND USES THEREOF; U.S. Provisional Patent Application No. 60/789,186, filed on Apr. 4, 2006, KERATOLYTIC ANTIFUNGAL FOAM; U.S. Provisional Patent Application No. 0/815948, filed on Jun. 23, 2006, entitled FOAMABLE COMPOSITIONS COMPRISING A CALCIUM CHANNEL BLOCKER, A CHOLINERGIC AGENT AND A NITRIC OXIDE DONOR; U.S. Provisional Patent Application No. 60/818,634, filed on Jul. 5, 2006, entitled DICARBOXYLIC ACID FOAMABLE VEHICLE AND PHARMACEUTICAL COMPOSITIONS THEREOF; U.S. Provisional Patent Application No. 60/843,140, filed on Sep. 8, 2006, entitled FOAMABLE VEHICLE AND VITAMIN PHARMACEUTICAL COMPOSITIONS THEREOF, all of which are incorporated herein by reference in their entirety. More particularly any of the active ingredients; the solvents; the surfactants; foam adjuvants; polymeric agents, penetration enhancers; preservatives, humectants; moisturizers; and other excipients as well as the propellants listed therein can be applied herein and are incorporated by reference.

The following examples further exemplify the benefit agent foamable pharmaceutical carriers, pharmaceutical compositions thereof, methods for preparing the same, and therapeutic uses of the compositions. The examples are for the purposes of illustration only and are not intended to be limiting of the invention. Many variations may be carried out by one of ordinary skill in the art and are contemplated.

Methodology

A general procedure for preparing foamable compositions is set out in WO 2004/037225, which is incorporated herein by reference.

Emulsion Foam

1. Mix oily phase ingredients and heat to 75° C. to melt all ingredients and obtain homogeneous mixture.
2. Mix polymers in water with heating or cooling as appropriate for specific polymer.
3. Add all other water soluble ingredients to water-polymer solution and heat to 75° C.
4. Add slowly internal phase to external phase at 75° C. under vigorous mixing and homogenize to obtain fine emulsion. Alternatively the external phase is added slowly to the internal phase.
5. Cool to below 40° C. and add sensitive ingredients with mild mixing.
6. Cool to room temperature.
   Waterless Foam
1. Dissolve the polymers in the main solvent with heating or cooling as appropriate for specific polymer. Add the all other ingredients and heat to 75° C. to melt and dissolve the various ingredients.
2. Cool to below 40° C. and add sensitive ingredients with mild mixing.
3. Cool to room temperature.
   Oily Waterless Foam
1. Mix all ingredients excluding polymers and heat to 75° C. to melt and dissolve and obtain homogeneous mixture.
2. Mix well and cool to below 40° C. and add the polymers and sensitive ingredients with moderate mixing.
3. Cool to room temperature.
   Oily Foam with Phospholipids and/or Water
1. Swell the phospholipids in the main oily solvent under mixing for at least 20 minutes until uniform suspension is obtained.
2. Add all other ingredients excluding polymers and heat to 75° C. to melt and dissolve and obtain homogeneous mixture.
3. Mix well and cool to below 40° C. and add the polymers and sensitive ingredients with moderate mixing.
4. Cool to room temperature.
5. In case of polymers dissolved in water or organic solvent, dissolve the polymers in the solvent with heating or cooling as appropriate for specific polymer and add to the oily mixture under vigorous mixing at ˜40° C.
   Production Under Vacuum

Optionally, the foamable formulation may be produced under nitrogen and under vacuum. Whilst the whole process can be carried out under an oxygen free environment, it can be sufficient to apply a vacuum after heating and mixing all the ingredients to obtain an emulsion or homogenous liquid. Preferrably the production chamber is equipped to apply a vacuum but if not the formulation can be for example placed in a dessicator to remove oxygen prior to filing and crimping.

Canisters Filling and Crimping

Each aerosol canister is filled with PFF and crimped with valve using vacuum crimping machine. The process of applying a vacuum will cause most of the oxygen present to be eliminated. Addition of hydrocarbon propellant may without being bound by any theory further help to reduce the likelihood of any remaining oxygen reacting with the active ingredient. It may do so, without being bound by any theory, by one or more of dissolving in the oil or hydrophobic phase of the formulation, by dissolving to a very limited extent in the aqueous phase, by competing with some oxygen from the formulation, by diluting out any oxygen, by a tendency of oxygen to occupy the dead space, and by oxygen occupying part of the space created by the vacuum being the unfilled volume of the canister or that remaining oxygen is rendered substantially ineffective in the formulation.

Pressurizing

Propellant Filling

Pressurizing is carried out using a hydrocarbon gas or gas mixture. Canisters are filled and then warmed for 30 sec in a warm bath at 50° C. and well shaken immediately thereafter.

Closure Integrity Test.

Each pressurized canister is subjected to bubble and crimping integrity testing by immersing the canister in a 60° C. water bath for 2 minutes. Canisters are observed for leakage as determined by the generation of bubbles. Canisters releasing bubbles are rejected.

Tests

By way of non limiting example the objectives of hardness, collapse time and FTC stability tests are briefly set out below as would be appreciated by a person of the art.

Hardness

LFRA100 instrument is used to characterize hardness. A probe is inserted into the test material. The resistance of the material to compression is measured by a calibrated load cell and reported in units of grams on the texture analyzer instrument display. Preferably at least three repeat tests are made. The textural characteristics of a dispensed foam can affect the degree of dermal penetration, efficacy, spreadability and acceptability to the user. The results can also be looked at as an indicator of softness. Note: the foam sample is dispensed into an aluminum sample holder and filled to the top of the holder.

Collapse Time

Collapse time (CT) is examined by dispensing a given quantity of foam and photographing sequentially its appearance with time during incubation at 36° C. It is useful for evaluating foam products, which maintain structural stability at skin temperature for at least 1 min.

Viscosity

Viscosity is measured with Brookfield LVDV-II+PRO with spindle SC4-25 at ambient temperature and 10, 5 and 1 RPM. Viscosity is usually measured at 10 RPM. However, at about the apparent upper limit for the spindle of ˜>50,000CP, the viscosity at 1 RPM may be measured, although the figures are of a higher magnitude.

FTC (Freeze Thaw Cycles)

To check the foam appearance under extreme conditions of repeated cycles of cooling, heating, (first cycle) cooling, heating (second cycle) etc., commencing with −100° C. (24 hours) followed by +400° C. (24 hours) measuring the appearance and again repeating the cycle for up to three times.

Creaming by centrifugation:

1. Principle of test

The centrifugation used in this procedure serves as a stress condition simulating the aging of the liquid dispersion under investigation. Under these conditions, the centrifugal force applied facilitates the coalescence of dispersed globules or sedimentation of dispersed solids, resulting in loss of the desired properties of the formulated dispersion.
   2. Procedure
2.1. Following preparation of the experimental formulation/s, allow to stand at room temperature for ≧24 h.
2.2. Handle pentane in the chemical hood. Add to each experimental formulation in a 20-mL glass vial a quantity of pentane equivalent to the specified quantity of propellant for that formulation, mix and allow formulation to stand for at least 1 h and not more than 24 h.
2.3. Transfer each mixture to 1.5 mL microtubes. Tap each microtube on the table surface to remove entrapped air bubbles.
2.4. Place visually balanced microtubes in the centrifuge rotor and operate the centrifuge at 3,000 rpm for 10 min or at 1,000 rpm for 10 min.
   Penetration—Protocol
   Objective

The study was designed to evaluate the skin permeability of a) non alcoholic emollient and b) waterless foam preparations comprising 5% and c) 30% ascorbic acid, and d) topical commercial/vitamin C serum with milicapsules (Holy Land Cosmetics—C The Success), which is also an non alcoholic formulation comprising up to 12% ascorbic acid (and 5% ascorbyl palmitate).

Experimental Procedure

Diffusion Cells

The permeability of porcine skin to ascorbic acid was measured in-vitro with a Franz diffusion cell system. The solutions on the receiver side were stirred by externally-driven, Teflon coated magnetic bars.

One experiment was performed using a diffusion cell system containing 12 cells. Cells 1-3 contained MP016 (5%), cells 4-6 contained AAP023 (30%), cells 7-9 contained AAP029 (5%), cells 10-11 contained Vitamin C serum and cell 12 as reference cell

Skin Preparation

Excised pig ear skin, approximately 4×4 cm, is supplied after dermatome sectioning at a thickness of ˜500 pm and stored at −18° C. until use.

Transepidermal water loss measurements (TEWL, Tewameter TM300) were performed and only those pieces that the TEWL levels were within specification (<15 g/m²h) were mounted in the diffusion cells and then the donor chambers were clamped in place. The receiver chamber, defined as the side facing the dermis, was filled with phosphate buffer (0.1M, pH 7.4)+0.2% sodium metabisulfite.

Permeation Study

Product specimens (200 mg) were applied on the skin.

0.5 mL were withdrawn from receiving chamber at T-0, 3, 6, 9, 12, 22 and 24 hours an inserted into 2-ml vials for rate study.

Tape-Stripping” Procedure for the Removal of Material Adhered to Skin

Adhere a piece of cello tape on each skin slice within the mold and overlay with 2 Kg weight for about 10 seconds. Remove the weight and transfer the tape into a 50 mL tube containing 10 mL receptor fluid

Repeat cello-tape adherence procedure for additional nine times (9 individual cell-tapes) and transfer all nine collected tapes to a 50 mL tube containing 3 mL receptor fluid. Repeat cello-tape adherence for additional ten times (10 individual cell-tapes) and transfer all ten tapes to a 50 mL tube containing 3 mL receptor fluid.

Skin extraction: Cut out the skin diffusion area (1.77 cm²) from the skin section, and transfer to a 5-mL tube containing 3 mL of the extracting solution.

The receiver and the skin extract solutions were transferred quantitatively into vials and analyzed by HPLC. The analyses were usually performed within two days from the sampling time.

Calculation

The permeating drug quantity per unit of the skin surface area was calculated by using the following formula:

According to requirements specified in the percutaneous absorption protocol, study results may be presented as shown below:
% API detected: for unabsorbed dose, % penetration (retention) of API and % permeation of API (accumulation in receptor solution)

$$A P I ( \% ) = \frac{A P I detected ( μg ) \times 100}{applied dose ( μg )}$$

Intra-Canister Uniformity
1. Representative product containers are collected, sample test solutions are prepared and the content of the analyte is determined according to standard methods in the art. Variability of content is characterized as percent difference or relative standard deviation, as appropriate, according to the number of samples evaluated.
2. The results ascertain variability or uniformity within a given container in content of analytes (primarily active pharmaceutical ingredients, but also preservatives) taken from different parts of a pressurized canister drug products
3. Two full canisters were shaken according to product instructions. About 1-3 g of Foam was dispensed from each canister and discarded. Foam sufficient for two replicate sample solution preparations was then dispensed into a glass beaker. This represents the initial sample. A middle portion is then dispensed from each canister being about half the canister contents. This middle dispensed portion may be discarded or collected for testing purposes, as necessary. Foam sufficient for two replicate sample solution preparations was then dispensed into a glass beaker. This represents the final sample. A small amount of formulation remains in the canister. The foam samples were stirred to remove gas/air bubbles. From both the initial and final foam portions from each canister 4 separate sample solutions are prepared and analyzed, 2 from the initial portion and 2 from the final portion. The percent difference is calculated as follows:

$$Difference between content determined in initial \& \frac{final portions}{Mean of content of initial \& final portions} \times 100$$
and the intra canister uniformity evaluated from the results.
   Stock Compositions

Non-limiting examples of how stock solutions are made up with and without API. Other stock solutions may be made using the same methodology by simply varying adding or omitting ingredients as would be appreciated by one of the ordinary skills in the art.

### EXAMPLES

The invention is described with reference to the following examples. For the purpose of the Examples below it was sufficient to apply a vacuum only at the crimping stage although for long term stability preferably any vacuum should be applied during manufacture as well at a sufficient pressure so that any oxygen remaining in the formulation is virtually negligible. This invention is not limited to these examples and experiments. Many variations will suggest themselves and are within the full intended scope of the appended claims.

### Part A

### Non Aqueous Formulations

### Example 1

### Foamable Carriers Containing Polyols and a Polymeric Agent

| | TECH PG- | TECH PG- | TECH PG- |
|---|---|---|---|
| | 014 | 015 | 016 |
| Ingredient | % W/W | % W/W | % W/W |
| Propylene glycol (PG) | 82.00 | 92.00 | 60.00 |
| Laureth-4 | 2.00 | 2.00 | 2.00 |
| Glyceryl stearate and | 4.00 | 4.00 | 3.00 |
| PEG-100 stearate | | | |
| (Simulsol 165) | | | |
| PEG 4000 | 10.00 | | |
| Glycerin anhydrous | | | 33.00 |
| Hydroxypropylcellulose | 2.00 | 2.00 | 2.00 |
| (Klucel EF) | | | |
| Total | 100.00 | 100.00 | 100.00 |

Notes:
The compositions are substantially non-aqueous
In order to create a foam, a propellant can be added at a concentration of about 3% to about 25%.
The compositions were shakable and produced foam of good quality.
Composition TECH PG-015 contains the minimum number of components that constitute a foamable composition, which upon release from an aerosol pressurized container affords foam of Good or Excellent quality. It contains a diol (PG), a polymeric agent (Klucel EF), and a non-ionic surface active agent (PEG-100 stearate and Laureth 4)
Composition TECH PG-014 demonstrates that the addition of 10% PEG (secondary polar solvent) maintains Good foam quality.
Composition TECH PG-016 demonstrates that a mixture of two polyols (PG and glycerin maintains Good foam quality. This composition possesses high skin hydration effect.

### Example 2

### Foamable Carriers Containing Polyols

| | TECH PG- | TECH PG- | TECH PG- |
|---|---|---|---|
| | 021 | 024 | 025 |
| Ingredient | % W/W | % W/W | % W/W |
| Propylene glycol (PG) | 91.00 | 58.00 | 43.00 |
| Stearyl alcohol | 2.00 | 1.00 | 1.00 |
| Laureth-4 | 2.00 | 2.00 | 2.00 |
| Glyceryl stearate and | 3.00 | 3.00 | 3.00 |
| PEG-100 stearate | | | |
| (Simulsol 165) | | | |
| Glycerin | | 33.00 | 33.00 |
| Hydroxypropylcellulose | 2.00 | 3.00 | 3.00 |
| (Klucel EF) | | | |
| Dimethyl isosorbide (DMI) | | | 15.00 |
| Total | 100.00 | 100.00 | 100.00 |

The following procedure was employed when the compositions of Example 2 were produced.

Step 1: Preparation of Phase A

1. Heat Propylene glycol and stearyl alcohol to 80-85° C.
2. Add Klucel while mixing.
3. Cool to 70-75° C. Add all other ingredients while mixing. Agitation continues until solution uniformity is reached
4. Cool solution to 30° C. with moderate mixing.
   Step 2: Canisters Filling and Crimping
1. Each aerosol canister 35×70 mm is filled with 30±5% g of the composition
2. Each canister was closed with an aerosol valve, using a vacuum crimping machine.
   Step 3: Pressurizing

Propellant (mix of propane, butane and isobutane) was added to each of the canisters.

Notes:
The compositions were shakable and produced foam of excellent quality.
Composition TECH PG-021, 24 and 25 demonstrates that the addition of 1-2% stearyl alcohol (foam adjuvant) facilitates the formation of foam with Excellent quality. Substituting Stearyl alcohol with stearic acid results in an excellent foam too.
Composition TECH PG-025 demonstrates that the addition of 15% DMI (foam adjuvant) facilitates the formation of foam with Excellent quality. This composition possesses high skin penetration enhancing properties.
In order to create a foam, a propellant can be added at a concentration of about 3% to about 25%.

### Example 3

### Foamable Carriers Containing Polyols

| | TECH PG- | TECH PG- | TECH PG- |
|---|---|---|---|
| | 026 | 027 | 028 |
| Ingredient | % W/W | % W/W | % W/W |
| Stearyl alcohol | 2.00 | 1.00 | 1.00 |
| Propylene glycol (PG) | 76.00 | 46.00 | 78.00 |
| Laureth-4 | 2.00 | 2.00 | 2.00 |
| Glyceryl stearate (and) | | 1.50 | |
| PEG-100 stearate | | | |
| (Simulsol 165) | | | |
| Glycerin anhydrous | | 33.00 | |
| Hydroxypropylcellulose | 2.00 | 1.50 | 1.50 |
| (Klucel EF) | | | |
| Dimethyl isosorbide (DMI) | 15.00 | 15.00 | 15.00 |
| Glyceryl stearate | 1.00 | | 1.00 |
| Ceteareth-6 (and) | 2.00 | | 1.50 |
| stearyl alcohol | | | |
| (Macrogol cetostearyl ether) | | | |
| Total | 100.00 | 100.00 | 100.00 |
| Foam quality | Excellent | Excellent | Excellent |

Notes:
Composition TECH PG-027 demonstrates that a mixture of two polyols (PG and glycerin, plus DMI (secondary polar solvent) maintains Excellent foam quality. This composition possesses high skin hydration effect. It further possesses high skin penetration enhancing properties.
In order to create a foam, a propellant can be added at a concentration of about 3% to about 25%.

### Example 4

### Additional Foamable Carriers Containing Polyols, Having Excellent Foam Structure

| | TECH-PG | TECH-PG | TECH-PG | TECH-PG | TECH-PG |
|---|---|---|---|---|---|
| | 029 | 030 | 031 | 032 | 033 |
| Ingredient | % w/w | % w/w | % w/w | % w/w | % w/w |
| Propylene Glycol | 91.0 | 58.0 | 43.0 | 46.0 | 78.0 |
| Stearyl Alcohol | 2.0 | 1.0 | 1.0 | 1.0 | 1.0 |
| Glycerin | — | 33.0 | 33.0 | 33.0 | — |
| Klucel EF | 2.0 | 3.0 | 3.0 | 1.5 | 1.5 |
| Laureth-4 | 2.0 | 2.0 | 2.0 | 2.0 | 2.0 |
| Simulsol 165 | 3.0 | 3.0 | 3.0 | 1.5 | — |
| Dimethyl Isosorbide | — | — | 15.0 | 15.0 | 15.0 |
| Macrogol Cetostearyl Ether | — | — | — | — | 1.5 |
| Glyceryl Stearate | — | — | — | — | 1.0 |

### Example 5

### Additional Foamable Carriers which can be Used for Vitamins with and without an Active Agent

| | Gel Phase | Gel Phase | Gel Phase | Gel Phase |
|---|---|---|---|---|
| Ingredient | 001 | 002 | 003 | 004 |
| Propylene glycol | 88.00 | 78.00 | 46.00 | 78.00 |
| Glycerin anhydrous | | | 33.00 | 10.00 |
| Stearyl Alcohol | 2.00 | 1.00 | 1.00 | 2.00 |
| Hydroxypropyl Cellulose | 2.00 | 1.50 | 1.50 | 2.00 |
| Laureth-4 | 2.00 | 2.00 | 2.00 | 2.00 |
| Glyceryl Monostearate/ | | | 1.50 | |
| PEG 100 Stearate | | | | |
| GMS NE | 2.00 | 1.00 | | 2.00 |
| Macrogol Cetostearyl | 1.00 | 1.50 | | 1.00 |
| ether | | | | |
| PPG-15 stearyl ether | 3.00 | | | 3.00 |
| Dimethyl isosorbide | | 15.00 | 15.00 | |
| Total: | 100.00 | 100.00 | 100.00 | 100.00 |

The polar solvents of the composition, including propylene glycol, glycerin and dimethyl isosorbide act as penetration enhancers for the vitamins and optional additional therapeutic agents.

Stock compositions were made from the above formulations, which were used as described below.

### Example 6

### Foamable Ascorbic Acid Compositions

Ascorbic acid was added to the carrier compositions of example 5, as follows.

| | WAS 001 | WAS 002 | WAS 003 | WAS 004 |
|---|---|---|---|---|
| Gel Phase Stock 001 | 95.00% | | | |
| Gel Phase Stock 002 | | 95.00% | | |
| Gel Phase Stock 003 | | | 95.00% | |
| Gel Phase Stock 004 | | | | 95.00% |
| Ascorbic Acid | 5.00% | 5.00% | 5.00% | 5.00% |
| Total: | 100.00 | 100.00 | 100.00 | 100.00 |

Notes:
In order to create a foam, a propellant can be added at a concentration of about 3% to about 25%.
Following addition of a propellant to the composition, foamable composition, which upon release from an aerosol pressurized container affords foam of Good or Excellent quality.
Following application of each of the foams on facial skin is favorable. The foam is easily spread and immediately absorbed into the skin with no extensive rubbing.

### Example 7

### Foamable Ascorbic Acid and Nicinamide Compositions

Ascorbic acid and niacinamide were concurrently added to the carrier compositions of example 5, as follows.

| | WAN 001 | WAN 002 | WAN 003 | WAN 004 |
|---|---|---|---|---|
| Gel Phase Stock 001 | 93.00% | | | |
| Gel Phase Stock 002 | | 93.00% | | |
| Gel Phase Stock 003 | | | 93.00% | |
| Gel Phase Stock 004 | | | | 93.00% |
| Ascorbic Acid | 5.00% | 5.00% | 5.00% | 5.00% |
| Niacinamide | 2.00% | 2.00% | 2.00% | 2.00% |
| Total: | 100.00 | 100.00 | 100.00 | 100.00 |

Notes:
In order to create a foam, a propellant can be added at a concentration of about 3% to about 25%.
Following addition of a propellant to the composition, foamable composition, which upon release from an aerosol pressurized container affords foam of Good or Excellent quality.
Following application of each of the foams on facial skin is favorable. The foam is easily spread and immediately absorbed into the skin with no extensive rubbing.

### Example 8

### Foamable Ascorbic Acid and Tocopheryl Acetate Compositions

Ascorbic acid and tocopheryl acetate were concurrently added to the carrier compositions of example 5, as follows.

| | WAT 001 | WAT 002 | WAT 003 | WAT 004 |
|---|---|---|---|---|
| Gel Phase Stock 001 | 94.00% | | | |
| Gel Phase Stock 002 | | 94.00% | | |
| Gel Phase Stock 003 | | | 94.00% | |
| Gel Phase Stock 004 | | | | 94.00% |
| Ascorbic Acid | 5.00% | 5.00% | 5.00% | 5.00% |
| Tocopheryl Acetate | 1.00% | 1.00% | 1.00% | 1.00% |
| Total: | 100.00 | 100.00 | 100.00 | 100.00 |

Notes:
In order to create a foam, a propellant can be added at a concentration of about 3% to about 25%.
Following addition of a propellant to the composition, foamable composition, which upon release from an aerosol pressurized container affords foam of Good or Excellent quality.

### Example 9

### Foamable Niacinamide Compositions

Niacinamide was added to the carrier compositions of example 5, as follows.

| | WNI 001 | WNI 002 |
|---|---|---|
| Gel Phase Stock 001 | 96.00% | |
| Gel Phase Stock 002 | | 96.00% |
| Niacinamide | 4.00% | 4.00% |
| Control: | 100.00 | 100.00 |

Notes:
In order to create a foam, a propellant can be added at a concentration of about 3% to about 25%.
Following addition of a propellant to the composition, foamable composition, which upon release from an aerosol pressurized container affords foam of Good or Excellent quality.

### Example 10

### Foamable Ascorbic Acid and Alpha Tocopherol Compositions

Ascorbic acid and alpha tocopherol were concurrently added to the carrier compositions of example 5, as follows.

| | WAT 001 | WAT 002 | WAT 003 | WAT 004 |
|---|---|---|---|---|
| Gel Phase Stock 001 | 94.00% | | | |
| Gel Phase Stock 002 | | 94.00% | | |
| Gel Phase Stock 003 | | | 94.00% | |
| Gel Phase Stock 004 | | | | 94.00% |
| Ascorbic Acid | 5.00% | 5.00% | 5.00% | 5.00% |
| Alpha Tocopherol | 1.00% | 1.00% | 1.00% | 1.00% |
| Total: | 100.00 | 100.00 | 100.00 | 100.00 |

Notes:
In order to create a foam, a propellant can be added at a concentration of about 3% to about 25%.
Following addition of a propellant to the composition, foamable composition, which upon release from an aerosol pressurized container affords foam of Good or Excellent quality.

### Example 11

### a) Foamable Polyols Compositions, Containing a Steroid Drug

The following steroids were included in formulations TECH-PG 30, 31 and 33 (see Example 4): betamethasone valerate 0.12%, clobetasol propionate 0.05%, betamethasone dipropionate 0.05%, fluocinolone acetonide 0.025%, hydrocortisone acetate 0.5% and hydrocortisone butyrate 0.1%. All samples were stored at 50° C. for 4 weeks, in order to assess their stability. The following table provides the results of this short-term stability study, which indicated high compatibility between the polyol composition and the steroid drugs, which are known to be temperature-sensitive.

| | % Degradation after | |
|---|---|---|
| | 4 weeks at 50° C. | |
| | TECH-PG 032 | TECH-PG 033 |
| Bethamethasone Valerate 0.12% | 1.8% | 1.7% |
| Clobetasol Propionate 0.05% | 4.2% | 5.0% |
| Bethamethasone Dipropionate 0.05% | 0 | 0 |
| Fluocinolone Acetonide 0.025% | 1.3% | 1.7% |
| Hydrocortisone Acetate 0.5% | 1.6% | 2.1% |
| Hydrocortisone Butyrate 0.1% | 2.6% | 2.8 |

See Example 4

### b) Prophetic Foamable Polyols Compositions, Containing a Vitamin and a Steroid Drug

Additionally, one or more of the following vitamins can be included in formulations TECH-PG 30, 31 and 33 (see Example 4): vitamin C (ascorbic acid) between 0.1 and 5% say, 0.1% 1%, 2% 3%, 4%, or 5%; vitamin C (magnesium ascorbyl phosphate) 3%, retinol 1%, retinoic acid 0.1%, niacinamide 2% and tocopherol 1% and Vitamin K. between 0.1 and 2% say, 0.1% or 1% or 2%, and are made up as indicted in Example 18.

### Example 12

### Foamable Polyol Pharmaceutical Composition Comprising a Combination of Betamethasone Dipropionate and Calcipotriol

| | FXCLB1 | FXCLB2 |
|---|---|---|
| Ingredient | % W/W | % W/W |
| Propylene glycol | 90.945 | 77.945 |
| Stearyl alcohol | 2.00 | 1.00 |
| Klucel EF | 2.00 | 1.50 |
| Laureth-4 | 2.00 | 2.00 |
| Simulsol 165 | 3.00 | |
| Macrogol Cetostearyl Ether | | 1.50 |
| Glyceryl Stearate | | 1.00 |
| Dimethyl isosorbide | | 15.00 |
| Calcipotriol | 0.005 | 0.005 |
| Betamethasone Dipropionate | 0.05 | 0.05 |

Notes:
Composition FXCLB1 and FXCLB2 contain two active agents (a corticosteroid and a vitamin D derivative, which are known to exert a synergistic therapeutic effect in psoriasis. These compositions contribute to enhanced skin penetration of the active agents.
The liquefied or gas propellant can be added at a concentration of about 3% to about 25%.

### Example 13

### Foamable Compositions Containing Polyethylene Glycol

| | % w/w | % w/w | % w/w | % w/w | % w/w | % w/w | % w/w |
|---|---|---|---|---|---|---|---|
| PEG400 | 87.50 | 91.50 | 87.50 | 89.50 | 87.50 | 87.50 | 87.50 |
| Klucel MX (hydroxypropyl cellulose) | 0.50 | 0 | 0.50 | 0 | 0.50 | 0 | 0.50 |
| Klucel LF (hydroxypropyl cellulose) | 0 | 0.50 | 0 | 0.50 | 0 | 0.50 | 0 |
| Lipocol C2 (POE (2) cetyl ether) | 2.00 | 2.00 | 0 | 0 | 0 | 0 | 0 |
| Myrj 52 | 0 | 0 | 2.00 | 2.00 | 0 | 0 | 0 |
| Steareth-2 | 0 | 0 | 0 | 0 | 2.00 | 2.00 | 0 |
| Dermofeel G10L (Polyglyceryl-10 | 0 | 0 | 0 | 0 | 0 | 0 | 2.00 |
| Laurate) | | | | | | | |
| Propellant | 10 | 6 | 10 | 8 | 10 | 10 | 10 |
| Density | 0.060 | 0.063 | 0.063 | 0.055 | 0.052 | 0.050 | 0.075 |

Notes:
The liquefied or gas propellant can be added at a concentration of about 3% to about 25%.
The foams of this example have a non-ionic surface active agent at a concentration of 2%. Total amounts of surface active agent foam adjuvant and polymeric agent is in the range of 2.5%.
The compositions are useful as carriers of various active therapeutic active agents.

### Example 14

### Foamable Hygroscopic Composition Containing Dimethyl Isosorbide

| | % w/w | % w/w | % w/w | % w/w |
|---|---|---|---|---|
| Oleyl alcohol | 2.50 | — | — | — |
| IPM | 5.00 | 5.00 | 5.00 | — |
| Caprylic/Capric Triglyceride | 5.00 | 5.00 | 5.00 | 46.00 |
| (MCT oil) | | | | |
| Epikuron P100 | — | — | — | 10.00 |
| PPG-15 stearyl ether | — | — | — | 2.00 |
| Sorbitane stearate | 8.00 | 8.00 | 8.00 | 2.00 |
| Glyceryl monostearate | — | 1.00 | 1.00 | 1.00 |
| Stearyl alcohol | — | 5.00 | 5.00 | — |
| Cetostearyl alcohol | 8.00 | — | — | — |
| Klucel MF | — | 0.50 | — | — |
| PVP K-90 | — | — | — | 0.50 |
| Sisterna SP50 | 5.00 | 8.00 | 8.00 | — |
| Propylene glycol | 2.50 | — | — | — |
| DMI | 55.50 | 59.00 | 59.50 | 20.00 |
| Water pure | — | — | — | 10.00 |
| Phenonip | 0.50 | 0.50 | 0.50 | 0.50 |
| Propellant | 8.00 | 8.00 | 8.00 | 8.00 |

### Example 15

### Comparison Between Polyethylene-Based Foamable Compositions with and without Gelling Agent

The compositions of the test articles are provided in the following table. All foams were dispensed on a warm surface (38° C.), and the time to full collapse of the foam was measured. As shown in the table, it has been strikingly demonstrated that foam compositions without a gelling agent exhibit a 100% breakdown within 30 seconds, while foams containing gelling agent remained, with and without surfactant, were stable for several minutes. This is relevant from the usability point of view, since a foam that is unstable at skin temperature cannot be applied to large areas affectively.

| | | | | | Formulation | |
|---|---|---|---|---|---|---|
| | Formulations | | | | with gelling | |
| | without gelling agent | | | | agent | |
| | PG33 | PG34 | PG35 | PG36 | TEC49 | PG29 |
| | % w/w | % w/w | % w/w | % w/w | % w/w | % w/w |
| PEG 400 | 87.25 | 93.00 | 91.00 | 92.00 | 90.50 | 93.50 |
| Klucel GE (gelling agent) | — | — | — | — | 0.50 | 0.50 |
| Ceteareth-16 | — | — | 2.00 | 1.00 | — | — |
| Emulsiying Wax NF | 1.80 | — | — | — | — | — |
| Steareth-10 | — | 0.40 | — | 0.50 | — | — |
| PEG-40 stearate | 1.35 | — | — | — | — | — |
| Steareth-2 | — | 0.60 | 1.00 | 0.50 | 1.00 | |
| Span 60 | 2.70 | — | — | — | — | — |
| Polysorbate 60 | 0.90 | — | — | — | — | — |
| Propellant | 6.00 | 6.00 | 6.00 | 6.00 | 8.00 | 6.00 |
| Collapse time (Seconds; | <30 | <30 | <30 | <30 | 240 | >300 |
| 38° C.) | | | | | | |

### Example 16

### Foamable Hygroscopic Composition Containing Polyethylene Glycol with No Surfactant

| | % w/w |
|---|---|
| PEG 400 | 93.50 |
| Klucel GF | 0.50 |
| Propellant (Butane/propane) | 6.00 |
| Foam quality | E |
| Density | 0.09 |

### Example 17

### Prophetic Foamable Vitamin Compositions with an Additional Therapeutic Agent

Foamable vitamin compositions are made up with. an active agent at either say 1%, 2%, 3%, 4%, 5%, 10%, 15%, 20%, 25%, 30% or more by weight of composition and added to any of the compositions illustrated in Examples 5-10 wherein the percentage amount of one or both polar solvents is reduced by an approximately equivalent amount by weight in the composition.

More particularly exemplary concentrations of additional therapeutic agents in foamable compositions are set out in Table 1. Each active agent is added into, for example, any of the carriers listed in any of Examples 5-10 above in a therapeutically effective concentration and amount. The methodology of addition is well known to those of the art. The composition is adjusted in each case so that it is made up to 100% w/w as appropriate by polar solvent.

TABLE 1
Exemplary Concentrations of Examples of Active Agents EXPAND

| Additional | Exemplary | |
|---|---|---|
| therapeutic agent | Concentration | Exemplary Use |
| Hydrocortisone acetate | 1% | Steroid responsive |
| Betamethasone valerate | 0.1% | inflammation and psoriasis or |
| Clobetasol propionate | 0.05% | atopic dermatitis |
| Acyclovir | 5% | Viral infection, herpes |
| Ciclopirox | 1% | Fungal infection, seborrhea, |
| | | dandruff, |
| Clindamycin | 2% | Bacterial infection, acne, |
| | | rosacea, |
| Azelaic acid | 15% | Acne, rosacea, |
| | | pigmentation disorder and |
| | | various dermatoses |
| Metronidazole | 0.25%-2% | Rosacea, bacterial infections |
| | | and parasite infestations |
| Diclofenac | 1% | Osteoarthritis, joint pain |
| Tacrolimus | 0.2% | Atopic dermatitis, eczema and |
| | | inflammation |
| Benzoyl peroxide | 1%-10% | Acne |
| Alpha-hydroxy acids | 1%-20% | Aging, wrinkles |
| Salicylic acid | 1%-10% | Acne |
| Hydroquinone | 1%-10% | Pigmentation disorders |
| Caffeine | 1%-10% | Cellulite |
| Coenzyme Q 10 | 0.1%-10% | Aging, pigmentation |

The above examples represent different drug classes and it is to be understood that other drugs belonging to each of the classes represented above may be included and used in the compositions in a safe and effective amount.

### Example 18

### Prophetic Foamable Carriers which can be Used for Vitamins with and without an Active Agent

a) Foamable vitamin carriers are made up with PEG or hexylene glycol or butylene glycol instead of glycerin anhydrous by weight of composition and added to any of the compositions illustrated in Examples 5-10
b) Foamable vitamin carriers are made up with PEG or hexylene glycol or butylene glycol instead of propylene glycol by weight of composition and added to any of the compositions illustrated in Examples 5-10

### Example 19

### Prophetic Foamable Vitamin Compositions with an Additional Active Agent

Foamable vitamin compositions are made up with. an active agent at either say 1%, 2%, 3%, 4%, 5%, 10%, 15%, 20%, 25%, 30% or more by weight of composition and added to any of the compositions illustrated in Example 19 wherein the percentage amount of one or both polar solvents is reduced by an approximately equivalent amount by weight in the composition. More particularly examples of additional active agents are as described in Example 19 above.

### Example 20

### 5% Ascorbic Acid, PEG or PG, and Surfactant or Polymeric Agent

| | AAP006 | AAP007 | AAP008 | AAP009 |
|---|---|---|---|---|
| Ingredient | | | | |
| PEG 200 | | | 93.00 | 93.00 |
| Propylene Glycol | 93.00 | 93.00 | | |
| (PG) | | | | |
| Steareth-2 | | 2.00 | 2.00 | |
| Klucel EF | 2.00 | | | 2.00 |
| Ascorbic acid | 5.00 | 5.00 | 5.00 | 5.00 |
| Total: | 100.00 | 100.00 | 100.00 | 100.00 |
| Propellant (1681) | 8.00 | 8.00 | 8.00 | 8.00 |
| [propane:iso- | | | | |
| butane:n- | | | | |
| butane] mixture | | | | |
| Appearance | | | | |
| Quality | Good | Good | Good | Good |
| Color | White | White | White | White |
| Odor | No Odor | No Odor | No Odor | No Odor |
| Shakability | Good | Good | Good | Good |
| Microscope | No | No crystals | No crystals | No |
| | crystals | | | crystals |
| Centrifugation 3K | | Homogenous | Homogenous | |
| Density (gr/ml) | 0.102 | 0.055 | 0.770 | 0.078 |
| Hardness (g) | 10.01 | 38.74 | 49.22 | 11.23 |
| Collapse time (sec.) | 70 | >300 | >300 | 30 |

Note:
All the formulations produced good quality foam without crystals
5% Ascorbic acid is solubilized in high levels of PEG or PG

### Example 21

### 5% Ascorbic Acid, PEG and Surfactant

| | AAP011 |
|---|---|
| Ingredient | |
| PEG 200 | 93.00 |
| Propylene Glycol | |
| PEG-40 stearate | 2.00 |
| Ascorbic acid | 5.00 |
| Total: | 100.00 |
| Propellant (1681) [propane:iso-butane:n- | 8.00 |
| butane] mixture | |
| Appearance | |
| Quality | Good |
| Color | White |
| Odor | No Odor |
| Shakability | Good |
| ph (1:5) | |
| Microscope | No |
| | crystals |
| Centrifugation 1K | Creaming |
| | 35% |
| Density (gr/ml) | 0.138 |
| Hardness (g) | 9.54 |
| Collapse time (sec.) | >300 |

Note:
Replacing PEG with PG resulted in poor foam

### Example 22

### 5% Ascorbic Acid, PEG or PG or DMI, and Surfactant with and without Polymeric Agent

| | AAP012 | AAP013 | AAP014 | AAP015 |
|---|---|---|---|---|
| Ingredient | | | | |
| PEG 200 | | | | 93.00 |
| Propylene Glycol | | 93.00 | | |
| DMI | 89.00 | | 91.00 | |
| Glyceryl | 2.00 | | 3.00 | |
| monostearate | | | | |
| Stearyl alcohol | 3.00 | | | |
| Steareth-21 | | 2.00 | | 2.00 |
| Klucel EF | 1.00 | | 1.00 | |
| Ascorbic acid | 5.00 | 5.00 | 5.00 | 5.00 |
| Total: | 100.00 | 100.00 | 100.00 | 100.00 |
| Propellant (1681) | 8.00 | 8.00 | 8.00 | 8.00 |
| [propane:iso- | | | | |
| butane:n- | | | | |
| butane] mixture | | | | |
| Appearance | | | | |
| Quality | Good | Good | Good | Good |
| Color | White | White | White | White |
| Odor | Faint Odor | No Odor | Faint Odor | No Odor |
| Shakability | Good | Good | Good | Good |
| ph (1:5) | | | | |
| Microscope | Crystals | No crystals | Crystals | No crystals |
| Density (gr/ml) | 0.077 | 0.095 | 0.157 | 0.098 |
| Hardness (g) | 14.58 | 11.82 | FG | 37.55 |
| Collapse time (sec.) | 40 | | | >300 |

Note:
All the formulations produced good quality foam.
5% Ascorbic acid is solubilized in high levels of PEG or PG but not in DMI

### Example 23

### 5%-7% Ascorbic Acid, PEG and Surfactant with a Fatty Alcohol but without Polymeric Agent

| | AAP019 | AAP020 | AAP021 | AAP029 |
|---|---|---|---|---|
| Ingredient | | | | |
| PEG 200 | | 91.00 | | |
| Propylene Glycol | 87.00 | | 91.00 | 92.50 |
| Oleyl alcohol | | | | 0.50 |
| Cetostearyl alcohol | 8.00 | | | |
| Steareth-2 | | 2.00 | 2.00 | 2.00 |
| Ascorbic acid | 5.00 | 7.00 | 7.00 | 5.00 |
| Total: | 100.00 | 100.00 | 100.00 | 100.00 |
| Propellant (1681) | 8.00 | 8.00 | 8.00 | 8.00 |
| [propane:iso- | | | | |
| butane:n- | | | | |
| butane] mixture | | | | |
| Appearance | | | | |
| Quality | Good | Good | Good | Good |
| Color | White | White | White | White |
| Odor | No Odor | No Odor | No Odor | No Odor |
| Shakability | Good | Good | Good | Good |
| Microscope | No | Crystals- | Crystals- | No |
| | crystals | PFF& Foam | PFF& Foam | crystals |
| Density (gr/ml) | 0.120 | | | |
| Hardness (g) | 35.98 | | | |
| Collapse time (sec.) | >300 | | | |

Note:
All the formulations produced good quality foam.
5% Ascorbic acid is solubilized but increasing the amount to 7% results in crystals appearing in the pre foam and foam compositions.

### Example 23A

### Quercitin and Rosmarinic Acid, PG and Surfactant with and without Ascorbic Acid

| Ingredient | CCP011 | CCP012 |
|---|---|---|
| propylene glycol | 96.80 | 91.80 |
| (PG) | | |
| steareth 2 | 2.00 | 2.00 |
| Rosmarinic acid | 0.20 | 0.20 |
| Quercetin | 1.00 | 1.00 |
| Ascorbic acid | | 5.00 |
| Total: | 100.00 | 100.00 |
| Propellant (5515) | 8.00 | 8.00 |
| propane butane and | | |
| isobutene mixture | | |
| Foam Quality | Excellent | Excellent |
| Foam Odor | No Odor | No Odor |
| Foam Shakability | Good | Good |
| Foam Color | Yellow | Yellow |
| PFF Color | Off white | Off white |
| Microscope | No | No |
| | Crystals | Crystals |

Note:
All the formulations produced excellent quality foam.
Potentially synergistic combination of quercitin and rosmarinic acid.
A significant excess of two types of reactive antioxidant flavonoids is provided to be available to react in place of vitamin C.

### Part B

### Aqueous Formulations

### Example 24

### 5% Ascorbic Acid Plus a-Tocopherol

| | AAP001 | AAP003 | AAP004 |
|---|---|---|---|
| Ingredient | | | |
| PPG-15 Stearyl Ether | 15.00 | 14.00 | 12.00 |
| Octyldodecanol | 12.00 | 11.00 | 10.00 |
| Oleyl alcohol | 10.00 | 10.00 | 9.00 |
| Cyclomethicone | 3.00 | 3.00 | 4.00 |
| Glyceryl monostearate | 6.00 | 6.00 | 5.00 |
| Stearyl alcohol | 6.00 | 6.00 | 6.00 |
| Cocoglycerides | 6.00 | 6.00 | 6.00 |
| Myristyl alcohol | 2.50 | 2.50 | 2.50 |
| Hydrogenated Castor Oil | 3.50 | 3.50 | 3.50 |
| Diisopropyl adipate | 10.00 | 10.00 | 9.00 |
| Aluminum starch | 10.00 | 7.00 | 7.00 |
| Octylensuccinate | | | |
| Ascorbic acid | 5.00 | 5.00 | 5.00 |
| a-Tocopherol | 1.00 | 1.00 | 1.00 |
| Water | 10.00 | 15.00 | 20.00 |
| Total: | 100.00 | 100.00 | 100.00 |
| Propellant (1681) [propane:iso- | 8.00 | 8.00 | 8.00 |
| butane:n-butane] mixture | | | |
| Appearance | | | |
| Quality | Good | Good | Good |
| Color | White | White | White |
| Odor | No Odor | No Odor | No Odor |
| Shakability | Good | Good | Good |
| Microscope | Crystals | Crystals | No |
| | | | crystals |

Note:
All the formulations produced good quality foam
By increasing the amount of water to about 20% eliminates crystals in the foam compositions.

### Example 25

### 5% Ascorbic Acid Plus Preservative

| | AAP005 |
|---|---|
| Ingredient | |
| Mineral oil light | 5.60 |
| Isopropyl myristate | 5.60 |
| Glyceryl monostearate | 0.45 |
| Stearyl alcohol | 0.85 |
| Polysorbate 80 | 0.90 |
| PEG-40 stearate | 2.60 |
| Xanthan gum | 0.30 |
| Avicel RC 581 | 2.00 |
| Sodium Citrate | 1.00 |
| Ascorbic acid | 5.00 |
| Disodium metabisulfite | 0.10 |
| EDTA disodium | 0.10 |
| Sharomix 824 | 1.00 |
| Water | 74.50 |
| Total: | 100.00 |
| Propellant (1681) [propane:iso- | 8.00 |
| butane:n-butane] mixture | |
| Appearance | |
| Quality | Excellent |
| Color | White |
| Odor | No Odor |
| Shakability | Good |
| ph (1:5) | 3.69 |
| Microscope | No |
| | crystals |
| Density (gr/ml) | 0.039 |
| Hardness (g) | 9.16 |
| Collapse time (sec.) | >300 |

### Example-26

### 5%-20% Ascorbic Acid Plus Preservative, Different Polymeric Agents and with and without Mineral Oil

| | AAP016 | AAP017 | AAP018 |
|---|---|---|---|
| Ingredient | | | |
| Mineral oil light | 11.00 | 11.00 | |
| Polysorbate 80 | 0.90 | | 2.00 |
| PEG-40 stearate | 2.60 | | |
| Sucrose ester 1811 | | 2.00 | |
| Xanthan gum | 0.30 | | |
| Avicel RC 581 | 2.00 | | 2.00 |
| CMC | | 0.50 | |
| Ascorbic acid | 5.00 | 5.00 | 20.00 |
| Disodium | 0.10 | 0.10 | 0.10 |
| metabisulfite | | | |
| Water | 78.10 | 81.40 | 75.90 |
| Total: | 100.00 | 100.00 | 100.00 |
| Propellant (1681) | 8.00 | 8.00 | 8.00 |
| [propane:iso-butane:n- | | | |
| butane] mixture | | | |
| Appearance | | | |
| Quality | Excellent | Good− | Excellent |
| Color | White | White | White |
| Odor | No Odor | No Odor | No Odor |
| Shakability | Good | Good | Good |
| ph (1:5) | 2.91 | | |
| Microscope | No crystals | No crystals | No crystals |
| Centrifugation 1K | Creaming 60% | Homogenous | Separation 30% |
| Centrifugation 3K | Creaming 20% | Creaming 40% | Sedimentation |
| | | | 40% |
| Density (gr/ml) | 0.034 | | 0.064 |
| Hardness (g) | 14.46 | | 10.08 |
| Collapse time (sec.) | >300 | | 190 |

Notes:
All the formulations produced good quality foam without crystals
Formulation 17 breaks quickly without mechanical stimulation

### Example-27

### 5%-35% Ascorbic Acid Plus Preservative and Polymer with and without Mineral Oil

| | AAP022 | AAP023 | AAP024 | AAP025 |
|---|---|---|---|---|
| Ingredient | | | | |
| Mineral oil light | | | 12.00 | |
| Polysorbate 80 | 2.00 | 2.00 | 3.00 | 2.00 |
| Avicel RC 581 | 2.00 | 2.00 | | 2.00 |
| Pemulen TR2 | | | 0.40 | |
| Ascorbic acid | 25.00 | 30.00 | 5.00 | 35.00 |
| Disodium metabisulfite | 0.50 | 0.60 | 0.10 | 0.70 |
| Water | 70.50 | 65.40 | 79.50 | 60.30 |
| Total: | 100.00 | 100.00 | 100.00 | 100.00 |
| Propellant (1681) | 8.00 | 8.00 | 8.00 | 8.00 |
| [propane:iso- | | | | |
| butane:n-butane] mixture | | | | |
| Appearance | | | | |
| Quality | Excellent | Excellent | Excellent | Excellent |
| Color | White | White | White | White |
| Odor | No Odor | No Odor | No Odor | No Odor |
| Shakability | Good | Good | Good | Good |
| Microscope | No | No | No | Crystals- |
| | crystals | crystals | crystals | PFF& |
| | | | | Foam |
| Hardness (g) | | 10.39 | 13.59 | 11.34 |

Notes:

All the formulations upto about 30% ascorbic acid produced good quality foam without crystals.

### Example-28

### Octyl Dodecanol and Isopropyl Myristate Silicone Compositions, with Preservative

| | AAP026 | AAP027 | AAP028 |
|---|---|---|---|
| Ingredient | | | |
| Octyl dodecanol | 6.00 | | 6.00 |
| Cyclomethicone | 1.00 | 1.00 | 1.00 |
| Isopropyl myristate | | 6.00 | |
| Glyceryl monostearate | 0.50 | 0.50 | 0.50 |
| Stearyl alcohol | 1.00 | 1.00 | 1.00 |
| Polysorbate 80 | 1.00 | 1.00 | |
| PEG-40 stearate | 2.50 | 2.50 | |
| Steareth-2 | | | 3.00 |
| Steareth-21 | | | 2.00 |
| Avicel RC 581 | 2.00 | 2.00 | 2.00 |
| Ascorbic acid | 5.00 | 5.00 | 5.00 |
| Disodium metabisulfite | 0.20 | 0.20 | 0.20 |
| EDTA disodium | 0.10 | 0.10 | 0.10 |
| Sharomix 824 | 1.00 | 1.00 | 1.00 |
| Water | 79.70 | 79.70 | 78.20 |
| Total: | 100.00 | 100.00 | 100.00 |
| Propellant (1681) | 8.00 | 8.00 | 8.00 |
| [propane:iso-butane:n- | | | |
| butane] mixture | | | |
| Appearance | | | |
| Quality | Excellent | Excellent | Excellent |
| Color | White | White | White |
| Odor | No Odor | No Odor | No Odor |
| Shakability | Good | Good | Good |
| ph (1:5) | | | |
| Microscope | No crystals | No crystals | No crystals |
| Density (gr/ml) | | | |
| Hardness (g) | 10.03 | 9.64 | |

Notes:
All the formulations produced good quality foam without crystals
Formulation 28 breaks quickly without mechanical stimulation

### Example-29

### Mineral Oil and Flavonoid Composition with and without Ascorbic Acid

| | AAP030 | AAP031 |
|---|---|---|
| Ingredient | | |
| Mineral oil light | 11.00 | 11.00 |
| Polysorbate 80 | 0.90 | 0.90 |
| PEG-40 stearate | 2.60 | 2.60 |
| Xanthan gum | 0.30 | 0.30 |
| Avicel RC 581 | 2.00 | 2.00 |
| Ascorbic acid | | 5.00 |
| Quercetin | 3.00 | 1.00 |
| Water | 80.20 | 77.20 |
| Total: | 100.00 | 100.00 |
| Propellant (1681) | 8.00 | 8.00 |
| [propane:iso-butane:n- | | |
| butane] mixture | | |
| Appearance | | |
| Quality | Excellent | Excellent |
| Color | yellow | yellow |
| Odor | No Odor | No Odor |
| Shakability | Good | Good |

Notes:
All the formulations produced excellent quality foam
It was possible to incorporate substantial amounts of flavonoid in the above formulations

### Example-30

### Rosmarinic Acid Composition with and without Ascorbic Acid and in Combination with Quercitin

| | AAP032 | AAP033 | AAP034 | AAP035 |
|---|---|---|---|---|
| Ingredient | | | | |
| Mineral oil light | 11.00 | | 11.00 | 11.00 |
| Propylene Glycol | | 92.80 | | |
| Polysorbate 80 | 0.90 | | 0.90 | 0.90 |
| PEG-40 stearate | 2.60 | | 2.60 | 2.60 |
| Steareth-2 | | 2.00 | | |
| Xanthan gum | 0.30 | | 0.30 | 0.30 |
| Avicel RC 581 | 2.00 | | 2.00 | 2.00 |
| Ascorbic acid | 5.00 | 5.00 | | |
| Rosmarinic acid | 0.20 | 0.20 | 0.20 | 0.20 |
| Quercetin | | | | 1.00 |
| Water | 78.00 | | 83.00 | 82.00 |
| Total: | 100.00 | 100.00 | 100.00 | 100.00 |
| Propellant (1681) | 8.00 | 8.00 | 8.00 | 8.00 |
| Appearance | | | | |
| Quality | Excellent | Good | Excellent | Excellent |
| Color | yellow | White | White | yellow |
| Odor | No Odor | No Odor | No Odor | No Odor |
| Shakability | Good | Good | Good | Good |
| Microscope | No | No | No | No |
| | crystals | crystals | crystals | crystals |

Notes:
All the formulations produced excellent quality foam without crystals
It was possible to incorporate substantial amounts of flavonoids in the above formulations

### Part C

### Microsponge® Prophetic Formulations

### Example-31

### Oil in Water Foam Formulation Comprising Microsponges Loaded with Active Agent

| | % w/w | % w/w |
|---|---|---|
| Ingredient | | |
| Drug Microsponge ® | 10 | 10 |
| Mineral oil | 5 | 15 |
| Isopropyl myristate | 5 | 15 |
| Glyceryl monostearate | 1 | 1 |
| Brij 72 | 3.00 | 3.00 |
| Brij 721 | 2.00 | 2.00 |
| Methocel E15 | 0.26 | 0.26 |
| Phenonip | 0.60 | 0.60 |
| Propellant | 8.00 | 8.00 |
| Water to | 100.00 | 100.00 |

Notes:
This prophetic formulation can be adapted for a range of low to Medium oil content oil-in-water emulsion foam.
The amount of microsponges may be varied from about 1% to about 25% of the formulation by increasing or decreasing the amount of the aqueous phase.
Any active agent suitable for loading in microsponges may be used, such as benzyl peroxide, tretinoin, hydroquinone and the like
In a preferred formulation the microsponges are loaded with one or more fat soluble vitamins or with one or more fat soluble flavonoids or combinations thereof
The liquefied or gas propellant can be added at a concentration of about 3% to about 25%.

### Example-32

### High Oil/Petrolatum in Water Foam Formulation Comprising Microsponges Loaded with Active Agent

Stock Formulation

| Ingredient | |
|---|---|
| White Petrolatum (sofmetic) | 42.00 |
| Mineral oil, light | 18.00 |
| Cetearyl alcohol | 2.00 |
| Ceteth-20 (Lipocol C-20) | 2.16 |
| Span 80 | 3.84 |
| Behenyl alcohol | 1.00 |
| Aluminum starch octenyl succinate | 3.00 |
| Citric acid | 0.18 |
| Sodium citrate | 0.14 |
| Water, purified | 27.48 |
| Sharomix 824 | 0.20 |
| Total | 100.00 |

| Ingredient | a | b | c | d |
|---|---|---|---|---|
| Stock | 90-00 | 95-00 | 75-00 | 85-00 |
| Microsponges | 10-00 | 15-00 | 25-00 | 15-00 |
| Total | 100-00 | 100-00 | 100-00 | 100-00 |
| Propellant | 10-00 | 10-00 | 10-00 | 10-00 |

Notes:
To the stock formulation [100-X}microsponges of X w/w % may be added say from about 1% to about 25% w/w of the formulation
This prophetic formulation can be adapted for a range of high oil content oil-in-water emulsion foam of upto about 90 oil phase.
Any active agent suitable for loading in microsponges may be used, such as benzyl peroxide, tretinoin, hydroquinone and the like
In a preferred formulation the microsponges are loaded with one or more vitamins or with one or more flavonoids or combinations thereof
The liquefied or gas propellant can be added at a concentration of about 3% to about 35%.

### Example-32

### Water in Oil Foam Formulation Comprising Microsponges Loaded with Active Agent

| Ingredient | % w/w |
|---|---|
| Drug Micosponge ® | 10.00 |
| Isopropyl Myristate | 400 |
| Lanolin | 4.50 |
| Beeswax | 1.80 |
| Glyceryl monostearate and PEG100 | 0.75 |
| stearate (Simulsol 165 Seppic) | |
| Glyceryl oleate | 0.70 |
| Arlatone 2121 (Uniquema) | 0.45 |
| Arlacel P135 (Uniquema) | 0.35 |
| Ceteth-2 (Lipocol C2) | 4.60 |
| Sucrose ester SP30 | 0.75 |
| Sucrose ester SP10 | 0.75 |
| Xanthan gum | 0.35 |
| Water purified | 30.00 |
| Disodium EDTA | 0.30 |
| Magnesium sulphate | 0.50 |
| Mineral oil light to | 100.00 |
| Propellant | 8.00 |

This prophetic formulation can be adapted for a range of high oil content water in oil emulsion foam of upto about 80% oil phase by reducing the water content.
The amount of microsponges may be varied from about 1% to about 25% of the formulation by increasing or decreasing the amount of the aqueous phase.
Any active agent suitable for loading in microsponges may be used, such as benzyl peroxide, tretinoin, hydroquinone and the like
In a preferred formulation the microsponges are loaded with one or more fat soluble vitamins or with one or more fat soluble flavonoids or combinations thereof
The liquefied or gas propellant can be added at a concentration of about 3% to about 25%.

### Example-33

### Oily Foam Formulation Comprising Microsponges Loaded with Active Agent

| Ingredient | % w/w |
|---|---|
| Drug Micosponge ® | 10.00 |
| Caprylic/Capric Triglyceride (MCT oil) | 47.50 |
| PPG-15 stearyl ether | 1.00 |
| Glyceryl monostearate | 2.00 |
| Stearyl alcohol | 4.70 |
| Sorbitan stearate | 2.00 |
| Epikuron P100 | 8.00 |
| PVP K-90 | 0.50 |
| Propylene glycol (PG) | 8.00 |
| Purified water | 8.00 |
| Sharomix 824 | 0.30 |
| Propellant | 8.00 |
| Control: | 100.00 |

This prophetic substantially non aqueous formulation can be adapted for a medium range of MCT oil content say plus or minus 10% oil by varying the water/PG content.
The amount of microsponges may be varied from about 1% to about 25% of the formulation by increasing or decreasing the amount of the oil, PG and aqueous phases.
Any active agent suitable for loading in microsponges may be used, such as benzyl peroxide, tretinoin, hydroquinone and the like
In a preferred formulation the microsponges are loaded with one or more vitamins or with one or more flavonoids or combinations thereof
The liquefied or gas propellant can be added at a concentration of about 3% to about 25%.

### Example-34

### Petrolatum Non Aqueous Foam Formulations Comprising Microsponges Loaded with Active Agent

| Ingredient | a % w/w | b % w/w | c % w/w |
|---|---|---|---|
| Drug Micosponge ® | 10.00 | 5.00 | 15.00 |
| Petrolatum, (sofmetic) | 75.00 | 83.00 | 73.00 |
| Mineral oil, light | 3.00 | 7-00 | 7-00 |
| PPG-15 stearyl ether | 5.00 | | |
| Lecithin | 5.00 | 5.00 | 5.00 |
| Sorbitan stearate | 2.00 | | |
| Control: | 100.00 | 100.00 | 100.00 |
| Propellant | 10.00 | 10.00 | 10.00 |

This prophetic formulation can be adapted for a high range of petrolatum content of upto about 90%.
The amount of microsponges may be varied from about 1% to about 25% of the formulation by increasing or decreasing the amount of the petrolatum
Any active agent suitable for loading in microsponges may be used, such as benzyl peroxide, tretinoin, hydroquinone and the like
In a preferred formulation the microsponges are loaded with one or more vitamins or with one or more flavonoids or combinations thereof
The liquefied or gas propellant can be added at a concentration of about 3% to about 35%.

### Example-35

### Hydrophilic Solvent Foam Formulation Comprising Microsponges Loaded with Active Agent

Foam

| | % w/w | % w/w |
|---|---|---|
| Drug Microsponge ® | 10 | 10 |
| Propylene glycol | To 100 | |
| PEG 400 | | To 100 |
| Ceteth 2 | 1 | 1 |
| HPMC | 0.5 | 0.53.00 |
| Propellant | 8.00 | 8.00 |

This prophetic formulation can be adapted for a high range of hydrophilic solvent content of upto about 95%.
The amount of microsponges may be varied from about 1% to about 25% of the formulation by increasing or decreasing the amount of the hydrophilic solvent
Any active agent suitable for loading in microsponges may be used, such as benzyl peroxide, tretinoin, hydroquinone and the like
In a preferred formulation the microsponges are loaded with one or more vitamins or with one or more flavonoids or combinations thereof
The liquefied or gas propellant can be added at a concentration of about 3% to about 35%.

### Example-36

### Liquid Wax Foam Formulation Comprising Microsponges Loaded with Active Agent

Stock A

| Ingredients | A1 |
|---|---|
| Jojoba Oil | 20 |
| Pemulen TR-2 | 0.62 |
| Polysorbate 80 | 3 |
| Purified water | 76.38 |
| Control: | 100 |

Stock B

| Ingredients | B1 |
|---|---|
| Isostearic acid | 30 |
| Caprilic/capric triglycerides | 2.5 |
| Oleyl alcohol | 7.5 |
| Sorbitan Stearate | 0.65 |
| Imiquimod | 5 |
| Benzyl alcohol | 2 |
| Xanthan gum | 0.35 |
| Hydroxypropyl methylcellulose | 0.35 |
| Glycerin | 2 |
| Polysorbate 60 | 2.5 |
| Methyl hydroxybenzoate | 0.2 |
| Propyl hydroxybenzoate | 0.02 |
| Purified water | 46.93 |
| Total: | 100 |

| Ingredient | a | b |
|---|---|---|
| Stock A or B | 90-00 | 95-00 |
| Microsponges | 10-00 | 15-00 |
| Total | 100-00 | 100-00 |
| Propellant | 10-00 | 10-00 |

This prophetic formulations can be adapted for a high range of liquid wax content of upto about 70%.
The amount of microsponges may be varied from about 1% to about 25% of the formulation by increasing or decreasing the amount of water and liquid waxes
Any active agent suitable for loading in microsponges may be used, such as benzyl peroxide, tretinoin, hydroquinone and the like
In a preferred formulation the microsponges are loaded with one or more vitamins or with one or more flavonoids or combinations thereof.
In the example active agent is present in the foam formulation and in the microsponges. Moreover, it is possible for the microsponges to have loaded the same and or other or different active agents.
The liquefied or gas propellant can be added at a concentration of about 3% to about 25%.

### Section D

### Pharmaceutical and Cosmetic Prophetic Formulations

### Example 37

### Exemplary Prophetic Foams Containing Active Pharmaceutical Ingredients (API)

Exemplary concentrations of active ingredients in foamable compositions are set out in Table 2 and in the following additional prophetic examples. Each active ingredient is added into, for example, any of the carriers listed in any of the above Examples in a therapeutically effective concentration and amount. The methodology of addition is well known to those of the art. The composition is adjusted in each case so that it is made up to 100% w/w by addition or reduction of one or more solvents as is appropriate to the active agents concerned.

A—Exemplary Concentration Ranges of Some APIs which are Addable to Foams

TABLE 2

| Class | Concentration | Exemplary Use |
|---|---|---|
| Hydrocortisone | 1% | Steroid responsive inflammation and |
| acetate | | |
| Betamethasone | 0.12% | psoriasis or atopic dermatitis |
| valerate | | |
| Clobetasol | 0.05% | |
| proprionate | | |
| Acyclovir | 5% | Viral infection, herpes |
| Ciclopirox | 1% | Fungal infection, seborrhea, dandruff, |
| Clindamycin | 1-2% | Bacterial infection, acne, rosacea, |
| Azelaic acid | 15% | Acne, rosacea, |
| | | pigmentation disorder and various |
| | | dermatoses |
| Metronidazole | 0.25%-2% | Rosacea, bacterial infections and |
| | | parasite infestations |
| Diclofenac | 1% | Osteoarthritus, joint pain |
| Tacrolimus | 0.2% | Atopic dermatitis, eczema and |
| | | inflammation |
| Caffeine | 5% | anti-cellulite |
| Clotrimazole | 1% | Fungal infection |
| Lidocaine base | 2% | Local anesthetic |
| Terbinafine HCL | 1% | Fungal infection |
| Gentamycin | 0.1% | Bacterial skin infections, burns or |
| | | ulcers |
| Dexpanthenol | 5% | Wounds, ulcers, minor skin infections |
| Urea | 5-10% | Emollient and keratolytic |
| | | Atopic dermatitis, eczema, ichthyosis |
| | | and hyperkeratotic skin disorders |
| Ammonium | 12%-17.5% | Dry scaly conditions of the skin |
| lactate | | including ichthyosis |
| Povidone-iodine | 10% | Antimicrobial —antiseptic |
| Benzoyl peroxide | 1%-10% | Acne |
| Alpha-hydroxy | 1%-20% | Aging, wrinkles |
| acids | | |
| Salicylic acid | 1%-10% | Acne |
| Hydroquinone | 1%-10% | Pigmentation disorders |
| calcipotriol | 0.005 | Psoriasis |
| calcitriol | 0.003 | Psoriasis |

B—Prophetic Steroid Compositions

The following steroids can be included in carriers, compositions and foams: betamethasone valerate 0.12%, clobetasol propionate 0.05%, betamethasone dipropionate 0.05%, fluocinolone acetonide 0.025%, hydrocortisone acetate 0.5% and hydrocortisone butyrate 0.1%.

C—Prophetic Vitamin and Steroid Compositions

Additionally, one or more of the following vitamins can be included in the carriers, compositions and foams: vitamin C (ascorbic acid) between 0.1 and 5% say, 0.1% 1%, 2% 3%, 4%, or 5%; vitamin C (magnesium ascorbyl phosphate) 3%, retinol 1%, retinoic acid 0.1%, niacinamide 2% and tocopherol 1% and Vitamin K. between 0.1 and 2% say, 0.1% or 1% or 2%; vitamin D, a derivative or analogue thereof such as calcipotriol between 0.001% to about 0.02% preferably 0.005%; calcitriol between about 0.001% to about 0.02% preferably about 0.003%; such as tacalcitol between about 0.001% to about 0.02% w/w preferably 0.004%.

D—Prophetic Vitamin Compositions with or without an Additional Therapeutic Agent

Foamable vitamin compositions at either say 0.1% 1%, 2%, 3%, 4%, or 5%, by weight of composition are made up with or without an active agent and added to any of the vehicles or compositions illustrated in the above Examples.

E—Different Drug Classes

All the above examples represent different drug classes and it is to be understood that other drugs belonging to each of the classes represented above or described elsewhere in the specification may be included and may be used in the compositions in a safe and effective amount.

### Section E

### Chemical Stability Test

### Example 39

### Stability of Ascorbic Acid in Aqueous Foam Formulations

| | AAP-016 | AAP023 |
|---|---|---|
| Mineral oil light | 11.00 | |
| PEG-40 stearate | 2.60 | |
| Xanthan gum | 0.30 | |
| Avicel RC 581 | 2.00 | 2.00 |
| Polysorbate 80 | 0.90 | 2.00 |
| Water pure | 78.00 | 65.40 |
| Ascorbic acid | 5.00 | 30.00 |
| Disodium metabisulfite | 0.20 | 0.60 |
| Total | 100.00 | 100.00 |
| Propellant | 8.00 | 8.00 |

| Sample name | Time point | Sample conc. (% w/w) |
|---|---|---|
| AAP016- | T-0 | 5.01 |
| | T-2w, 25° C. | 5.00 |
| | T-2w, 40° C. | 5.18 |
| | T-2w, sun* | 4.79 |

| Sample name | Time point | Sample conc. (% w/w) |
|---|---|---|
| AAP023 | T-0 | 29.75 |
| | T-2w, 25° C. | 30.79 |
| | T-2w, 40° C. | 30.51 |
| | T-2w, sun* | 29.07 |

*glass vial with pre foam formulation (PFF) + 8% pentane located in the sun.

Note:
No significant breakdown was seen in any of the samples save possibly in the sun exposed samples.
See Examples 26 and 27 above for physical parameters.

### Example 40

### Stability of Ascorbic Acid in Non-Aqueous Foam Formulations

| | AAP029 | AAP007 |
|---|---|---|
| Propylene Glycol | 92.50 | 93.00 |
| Oleyl alcohol | 0.50 | 2.00 |
| Steareth-2 | 2.00 | |
| Ascorbic acid | 5.00 | 5.00 |
| Total | 100.00 | 5.00 |
| Propellant (1681) | 8.00 | 8.00 |

| | | Sample conc. |
|---|---|---|
| Sample name | Time point | (% w/w) |
| AAP007- | T-0 | 5.00 |
| | T-2w, 250 C | 5.10 |
| | T-2w, 400 C | 5.09 |
| | T-2w, sun* | 5.50 |

| | | Sample |
|---|---|---|
| | | conc. (% |
| Sample name | Time point | w/w |
| AAP029- | T-0 | 5.14 |
| | T-2w, 25° C. | 4.80 |
| | T-2w, 40° C. | 4.83 |
| | T-2w, sun* | 4.88 |

*glass vial with PFF + 8% pentane located in the sun.

Note:
No breakdown of substance was seen in any of the samples.
See Examples 20 and 23 above for physical parameters

### Section F

### Permeation Test

### Example 41

### Penetration of Ascorbic Acid from Aqueous and Non Aqueous Formulations In and Through Skin

See Methodology for the protocol. Formulations 16, 23, and 29 below appear in Examples 26, 27 and 23 respectively as well as in Examples 39 and 40 above.

Average in % of applied dose

| | | | | Vitamin C |
|---|---|---|---|---|
| | AAP016- | AAP023- | AAP029- | serum* |
| | (n = 3) | (n = 3) | (n = 3) | (n = 2) |
| Vit C | 5% | 30% | 5% | 12% (plus 5% |
| | | | | ascorbyl |
| | | | | palmitate) |
| Aqueous | Yes | Yes | No | Yes |
| Tape 1 | 70.45 | 39.48 | 82.18 | 53.24 |
| Tape 2 | 3.45 | 1.38 | 1.48 | 1.06 |
| Tape 3 | 0.34 | 0.54 | 0.20 | 0.08 |
| SE | 1.90 | 2.21 | 0.40 | 1.19 |
| RC (as is) | 13.23 | 33.96 | 3.64 | 23.01 |
| Total Stratum | 3.79 | 1.92 | 1.68 | 1.14 |
| Corneum (Tapes | | | | |
| 2, 3) | | | | |
| Total without tape 1 | 18.92 | 38.09 | 5.72 | 25.34 |
| (Tape 2, 3, SE and | | | | |
| RC) | | | | |
| Total skin | 5.69 | 4.13 | 1.08 | 2.33 |
| (Tape 2, 3 and SE) | | | | |
| Total Mass | 89.37 | 77.56 | 87.90 | 78.58 |
| balance | | | | |

*Commercial cosmetic product
*Tape 1 = Surface; Tape 2 = Upper Stratum Corneum; Tape 3 = Lower Stratum Corneum; SE = is the extract of the skin remaining after removal of Tapes 1, 2, and 3, and includes the hair follicle root.

Comment:

The penetration study results (presented as a mean of “n” repeat experiments) may indicate that

a) The aqueous formulations prepared as described in Examples 26 and 27 have about double the penetration in whole skin compared to that seen in the comparative aqueous commercial preparation.
b) Increased loading of vitamin C in the aqueous formulations did not appear to affect the amount found within the whole skin, however increased loading did result in higher levels passing through the skin indicating that some transport mechanism being operative.
c) All the formulations prepared as described in Examples 26, 27 and 23 resulted in higher penetration into the stratum corneum.
d) The non aqueous formulation as described in Example 23 disclosed the lowest levels in the remaining skin and of penetration through the skin. Without being bound by any theory, water and or minerals may play a role in transport and the absence thereof retards penetration except to an extent in the stratum corneum. The waterless formulation may be useful where delivery of the Vitamin, flavonoid or other agent is not desirable systemically and is only required in the stratum corneum.

What is claimed is:

1. A foamable hygroscopic vitamin and or flavonoid containing composition comprising:
a. at least one agent selected from the group consisting of vitamins and flavonoids; and
b. at least one hygroscopic solvent at a sufficient concentration to provide an Aw value of the hygroscopic vitamin and or flavonoid containing composition of less than 0.9;
c. a foam stabilizer selected from the group consisting of at least one surface-active agent; and at least one polymeric agent selected from the group consisting of a bioadhesive agent, a gelling agent, a film forming agent and a phase change agent and mixtures thereof; and
d. a hydrocarbon propellant at a concentration of about 3% to about 25% by weight of the total composition; and
wherein the composition is contained in a pressurized container to which a vacuum was applied prior to filling with a hydrocarbon propellant, is substantially flowable and provides a foam upon release;
wherein applying a vacuum to and filling a canister containing the composition with hydrocarbon propellant contributes to stabilize the agent against degradation.

2. The composition of claim 1, wherein the vitamin is selected from the group consisting of vitamin A, B1, B2, B3, B5, B6, B7, B9, B12, PABA, C, D1, D2, D3, D4, D5, E, K and F combinations thereof.

3. The composition of claim 1, wherein the vitamin or flavonoid is soluble in water.

4. The composition of claim 1, wherein the vitamin or flavonoid is soluble in a hydrophobic solvent.

5. The composition of claim 1, wherein the vitamin is a combination of two or more vitamins selected from the group consisting of vitamin A, B3, C, K, E, and F.

6. The composition of claim 1, wherein the vitamin is a combination of two or more vitamins selected from the group consisting of vitamin A, C, D1, D2, D3, D4, and D5.

7. The composition of claim 1, wherein the flavonoid is selected from the group consisting of flavonols; flavones flavonones flavan-3-ols anthocyanins and proanthocyanidins.

8. The composition of claim 1, wherein the flavonoid is a single flavonoid, a combination of two or more flavonoids or a synergistic combination of two or more flavonoids and is selected from the group consisting of benzquercin, diosmin, ethoxazorutoside, flavodate, sodium hesperidin, leucocianido, monoxerutin, oxerutin, quercetin, rutoside, rosmarinic acid, kaempferol, myricetin apigenin, luteolin, hesperetin, naringenin, eriodictyol, cyanidin, delphinidin, malvidin, pelargonidin, peonidin, petunidin, catechin, gallocatechin, epicatechin, and epigallocatechin

9. The composition of claim 1, wherein the flavonoid is quercetin and or rosmarinic acid and the vitamin is vitamin A, and or C and or D (D1, D2, D3, D4, or D5) and or E.

10. The composition of claim 1, wherein the vitamin is a combination of two or more vitamins selected from the group comprising vitamin B3, E and C and a derivative thereof.

11. The composition of claim 1 wherein the vitamin, flavonoid or a derivative thereof or combinations thereof improves, stimulates or promotes target site metabolism.

12. The composition of claim 1, further comprising a preservative and/or modulating agent and/or an anti oxidant.

13. The composition of claim 12, wherein the preservative is selected from the group consisting of disodium metabisulfate and sharomix 824 and the modulating agent is EDTA and or optionally a buffering agent.

14. The composition of claim 1, further comprising an additional therapeutic agent.

15. The composition of claim 1, wherein the hygroscopic solvent is selected from the group consisting of:
a. polyethylene glycols (PEGS);
b. surfactants comprising PEG;
c. polyols;
d. monosaccharides, disaccharides, oligosaccharides and sugar alcohols in an amount to provide hygroscopic properties; and
e. honey.

16. The foamable composition of claim 15, wherein the polyol is selected from the group consisting of a diol, a triol and a saccharide,
wherein the diol is selected from the group consisting of propylene glycol, butanediol, butenediol, butynediol, pentanediol, hexanediol, octanediol, neopentyl glycol, 2-methyl-1,3-propanediol, diethylene glycol, triethylene glycol, tetraethylene glycol, dipropylene glycol and dibutylene glycol and, wherein the triol is selected from the group consisting of glycerin, butane-1,2,3-triol, butane-1,2,4-triol and hexane-1,2,6-triol.

17. The foamable composition of claim 15, wherein the PEG is selected from the group consisting of PEG 200, PEG 300, PEG 400, PEG 600, PEG 1000, PEG 4000, PEG 6000 and PEG 8000.

18. The foamable composition of claim 15, wherein the hygroscopic solvent comprises a hygroscopic solvent mixture of at least one polyol and at least one PEG.

19. The foamable composition of claim 15, which is non aqueous or substantially non aqueous, also comprising wherein the hygroscopic solvent comprises (1) at least one hygroscopic solvent selected from a diol, a triol and PEG, and (2) at least a secondary hygroscopic solvent, which is selected from the group consisting of dimethyl isosorbide, tetrahydrofurfuryl alcohol polyethyleneglycol, ether, DMSO, a pyrrolidone, N-Methyl-2-pyrrolidone, 1-Methyl-2-pyrrolidinone, ethyl proxitol, dimethylacetamide, a PEG-type surfactant, an alpha hydroxy acid, lactic acid and glycolic acid.

20. The composition of claim 1, wherein the Aw value of the composition is selected from the ranges of (1) about 0.8 and about 0.9; (2) about 0.7 and about 0.8; and (3) less than about 0.7.

21. The composition of claim 1, further comprising up to 10% of water.

22. The composition of claim 1, wherein the composition is substantially non-aqueous and/or substantially alcohol-free.

23. The composition of claim 1, comprising a vitamin, a flavonoid and or a conservational agent, a stabilizer, and solvent comprising water, wherein the flavonoid and or conservational agent can substantially conserve or stabilize the vitamin or ameliorate, reduce or inhibit its breakdown and wherein the vitamin, flavonoid, stabilizer, conservational agent, and solvent provide a composition that is substantially resistant to aging.

24. The composition of claim 1, wherein the vitamin and or flavonoid or derivative thereof or combinations thereof are loaded into microsponges.

25. A foamable composition comprising a stabilized-vitamin, comprising:
a water-unstable vitamin;
an antioxidative flavonoid; and
a foamable carrier comprising:
i. water:
ii. a foam stabilizer selected from the group consisting of at least one surface-active agent; and at least one polymeric agent selected from the group consisting of a bioadhesive agent, a gelling agent, a film forming agent and a phase change agent and mixtures thereof;
iii. about 0.005% to about 5% conservational agent; and
iv. a propellant at a concentration of about 3% to about 25% by weight of the total composition,
where said foamable carrier reduces the destabilizing effect of water on the vitamin,
wherein the composition is contained in a pressurized container, is substantially flowable and provides a foam upon release.

26. The foamable composition of claim 25, wherein the foamable carrier further comprises a solvent selected from the group consisting of a hydrophilic solvent, a hydrophobic solvent, a polar solvent, a silicone, an emollient and mixtures thereof.

27. The foamable composition of claim 25, wherein the vitamin is selected from the group consisting of vitamin A, B1, B2, B3, B5, B6, B7, B9, B12, PABA, C, D1, D2, D3, D4, D5, E, K and F and a derivative thereof or combinations thereof.

28. The foamable composition of claim 25, wherein the vitamin or flavonoid is soluble in water.

29. The foamable composition of claim 25, wherein the vitamin or flavonoid is soluble in a hydrophobic solvent.

30. The foamable composition of claim 25, wherein the vitamin is a combination of two or more vitamins selected from the group consisting of vitamin A, B3, C, K, E, and F and a derivative thereof.

31. The foamable composition of claim 25, wherein the vitamin is a combination of two or more vitamins selected from the group consisting of vitamin A, C, D1, D2, D3, D4, and D5 and a derivative thereof.

32. The foamable composition of claim 25, wherein the flavonoid is selected from the group consisting of flavonols; flavones flavonones flavan-3-ols anthocyanins and proanthocyanidins.

33. The foamable composition of claim 25, wherein the flavonoid is a single flavonoid, a combination of two or more flavonoids or a synergistic combination of two or more flavonoids and is selected from the group consisting of benzquercin, diosmin, ethoxazorutoside, flavodate, sodium hesperidin, leucocianido, monoxerutin, oxerutin, quercetin, rutoside, rosmarinic acid, kaempferol, myricetin apigenin, luteolin, hesperetin, naringenin, eriodictyol, cyanidin, delphinidin, malvidin, pelargonidin, peonidin, petunidin, catechin, gallocatechin, epicatechin, and epigallocatechin.

34. The foamable composition of claim 25, wherein the flavonoid is quercetin and or rosmarinic acid and the vitamin is vitamin A, and or C and or D (D1, D2, D3, D4, or D5) and or E or a derivative thereof.

35. The foamable composition of claim 25, wherein the vitamin is a combination of two or more vitamins selected from the group comprising vitamin B3, E and C and a derivative thereof.

36. The composition of claim 25 wherein the vitamin, flavonoid or a derivative thereof or combinations thereof improves, stimulates or promotes target site metabolism.

37. The foamable composition of claim 25, further comprising a preservative and or modulating agent and or an anti oxidant.

38. The foamable composition of claim 37 wherein the preservative is selected from the group consisting of disodium metabisulfate and sharomix 824 and the modulating agent is EDTA and or optionally a buffering agent.

39. The foamable composition of claim 26, wherein the polar solvent is a polyol is selected from the group consisting of a diol, a triol and a saccharide.
wherein the diol is selected from the group consisting of propylene glycol, butanediol, butenediol, butynediol, pentanediol, hexanediol, octanediol, neopentyl glycol, 2-methyl-1,3-propanediol, diethylene glycol, triethylene glycol, tetraethylene glycol, dipropylene glycol and dibutylene glycol and, wherein the triol is selected from the group consisting of glycerin, butane-1,2,3-triol, butane-1,2,4-triol and hexane-1,2,6-triol.

40. The foamable composition of claim 26, wherein the polar solvent is a PEG is selected from the group consisting of PEG 200, PEG 300, PEG 400, PEG 600, PEG 1000, PEG 4000, PEG 6000 and PEG 8000.

41. The foamable composition of claim 26, wherein the carrier composition comprises a polar solvent mixture of at least one polyol and at least one PEG.

42. The foamable composition of claim 26, which is non aqueous or substantially non aqueous, also comprising wherein the hygroscopic solvent comprises (1) at least one hygroscopic solvent selected from a diol, a triol and PEG, and (2) at least a secondary hygroscopic solvent, which is selected from the group consisting of dimethyl isosorbide, tetrahydrofurfuryl alcohol polyethyleneglycol, ether, DMSO, a pyrrolidone, N-Methyl-2-pyrrolidone, 1-Methyl-2-pyrrolidinone, ethyl proxitol, dimethylacetamide, a PEG-type surfactant, an alpha hydroxy acid, lactic acid and glycolic acid.

43. The foamable composition of claim 26, wherein the polymeric agent is selected from the group consisting of locust bean gum, sodium alginate, sodium caseinate, egg albumin, gelatin agar, carrageenin gum, sodium alginate, xanthan gum, quince seed extract, tragacanth gum, guar gum, cationic guars, hydroxypropyl guar gum, starch, an amine-bearing polymer, chitosan, alginic acid, hyaluronic acid, a chemically modified starch, a carboxyvinyl polymer, polyvinylpyrrolidone, polyvinyl alcohol, a polyacrylic acid polymer, a polymethacrylic acid polymer, polyvinyl acetate, a polyvinyl chloride polymer, a polyvinylidene chloride polymer, methylcellulose, hydroxypropyl cellulose, hydroxypropyl methylcellulose, hydroxyethyl cellulose, hydroxy propylmethyl cellulose, methylhydroxyethylcellulose, methylhydroxypropylcellulose, hydroxyethylcarboxymethylcellulose, carboxymethyl cellulose, carboxymethylcellulose carboxymethylhydroxyethylcellulose, a cationic cellulose PEG 1000, PEG 4000, PEG 6000 and PEG 8000 of Carbopol® 934, Carbopol® 940, Carbopol® 941, Carbopol® 980, Carbopol® 981. hydroxypropylcellulose and carbomer.

44. The foamable composition of claim 25, wherein the surface active agent is selected from the group consisting of a polysorbate, polyoxyethylene (20) sorbitan monostearate, polyoxyethylene (20) sorbitan monooleate, a polyoxyethylene fatty acid ester, Myrj 45, Myrj 49, Myrj 52 and Myrj 59, a polyoxyethylene alkylyl ether, polyoxyethylene cetyl ether, polyoxyethylene palmityl ether, polyethylene oxide hexadecyl ether, polyethylene glycol cetyl ether, brij 38, brij 52, brij 56 brij 72, brij 721 and brij W1, a sucrose ester, a partial ester of sorbitol, sorbitan monolaurate, sorbitan monolaurate a monoglyceride, a diglyceride, isoceteth-20, a sucrose ester, or selected from the group consisting of steareth 2, glyceryl monostearate/PEG 100 stearate, Glyceryl Stearate, Steareth-21, peg 40 stearate, polysorbate 60, polysorbate 80, sorbitan stearate, laureth 4, Sorbitan monooleate, ceteareth 16 ceteareth 20, steareth 10, steareth 20, ceteth 20, Macrogol Cetostearyl Ether, ceteth 2, PEG-30 Dipolyhydroxystearate, sucrose distearate, polyoxyethylene (100) stearate, PEG 40 stearate, PEG 100 stearate, laureth 4, cetomacrogol ether, Cetearyl alcohol, Cetearyl glucoside, Oleyl alcohol, Steareth-2, Diisopropyl adipate, Capric/caprilic triglicerides, Polysorbate 20; Polysorbate 80, Montanov 68 (CETEARYL ALCOHOL (and) CETEARYL GLUCOSIDE), Simusol 165 (Glyceryl stearate and PEG-100 stearate). Methyl glucose sequistearate, Peg 30 dipolyhydroxystearate, sucrose stearic acid esters, sorbitan laureth, sorbitan stearate, polyglyceryl-10 laurate, epikuuron 80, span 80 and mixtures thereof.

45. The foamable composition of claim 26, further comprising a foam adjuvant selected from the group consisting of a fatty alcohol, a fatty acid and a hydroxyl fatty acid or liquid wax.

46. The foamable composition of claim 25, further comprising at least one additional therapeutic agent in addition to the vitamin and flavonoid or combinations thereof in a therapeutically effective concentration.

47. The foamable composition of claim 25, wherein the vitamin and flavonoid or combinations thereof are provided in a microsponge.

48. The foamable composition of claim 25, further comprising at least one additional therapeutic agent selected from the group consisting of active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, wound healing agents and wart removers.

49. The foamable composition of claim 46, wherein the at least one additional therapeutic agent is an anti-infective agent.

50. The composition of claim 25, comprising a vitamin, a flavonoid and or a conservational agent, a stabilizer, and solvent comprising water,
wherein the flavonoid and or conservational agent can substantially conserve or stabilize the vitamin or ameliorate, reduce or inhibit its breakdown and
wherein the vitamin, flavonoid, stabilizer, conservational agent, and solvent provide a composition that is substantially resistant to aging.

51. A method of treating a disorder or condition of mammalian subject, comprising:
administering a foam produced from a foamable vitamin and or flavonoid composition to a target site, the composition comprising:
a foamable composition comprising a stabilized-vitamin, comprising:
a therapeutically effective amount of a water-unstable vitamin;
an optional flavonoid; and
a foamable carrier comprising:
i. water:
ii. a foam stabilizer selected from the group consisting of at least one surface-active agent; at least one polymeric agent selected from the group consisting of a bioadhesive agent, a gelling agent, a film forming agent and a phase change agent and mixtures thereof;
iii. about 0.005% to about 5% conservational agent; and
iv. a propellant at a concentration of about 3% to about 25% by weight of the total composition,
where said foamable carrier reduces the destabilizing effect of water on the vitamin,
wherein the composition is contained in a pressurized container, is substantially flowable and provides a foam upon release.

52. The method of claim 51, wherein the vitamin and or flavonoid or derivative thereof or combinations thereof are loaded into microsponges.

53. The method of claim 51 further comprising an additional therapeutic agent in addition to the vitamin, flavonoid or combinations thereof.

54. The method of claim 51, wherein the target site is selected from the group consisting of the skin, a body cavity, a mucosal surface, the nose, the mouth, the eye, the ear canal, the respiratory system, the vagina and the rectum.

55. The method of claim 51, wherein the disorder is selected from the group consisting of dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, ecthyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum; and wherein the active agent is suitable for treating said disorder.

56. The method of claim 53, wherein the at least one additional therapeutic agent is selected from the group consisting of active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, wound healing agents and wart removers.

57. The method of claim 53, wherein the at least one active agent is selected from the group consisting of alclometasone dipropionate, amcinafel, amcinafide, amcinonide, beclomethasone, beclomethasone dipropionate, betamethsone, betamethasone benzoate, betamethasone dexamethasone-phosphate, dipropionate, betamethasone valerate, budesonide, chloroprednisone, chlorprednisone acetate, clescinolone, clobetasol, clobetasol propionate, clobetasol valerate, clobetasone, clobetasone butyrate, clocortelone, cortisone, cortodoxone, craposone butyrate, desonide, desoxymethasone, dexamethasone, desoxycorticosterone acetate, dichlorisone, diflorasone diacetate, diflucortolone valerate, difluorosone diacetate, diflurprednate, fluadrenolone, flucetonide, flucloronide, fluclorolone acetonide, flucortine butylesters, fludroxycortide, fludrocortisone, flumethasone, flumethasone pivalate, flumethasone pivalate, flunisolide, fluocinolone, fluocinolone acetonide, fluocinonide, fluocortin butyl, fluocortolone, fluorometholone, fluosinolone acetonide, fluperolone, fluprednidene acetate, fluprednisolone hydrocortamate, fluradrenolone, fluradrenolone acetonide, flurandrenolone, fluticasone, halcinonide, halobetasol, hydrocortisone, hydrocortisone acetate, hydrocortisone butyrate, hydrocortisone cyclopentylpropionate, hydrocortisone valerate, hydroxyltriamcinolone, medrysone, meprednisone, .alpha.-methyl dexamethasone, methylprednisolone, methylprednisolone acetate, mometasone furoate, paramethasone, prednisolone, prednisone, pregnenolone, progesterone, spironolactone, triamcinolone, triamcinolone acetonide and derivatives, esters and salts thereof.

58. The method of claim 53, wherein the at least one additional therapeutic agent is an anti-infective agent.

59. The method of claim 53, wherein the at least one anti-infective agent is selected from the group consisting of an antibiotic agent, an antibacterial agent, an antifungal agent, an agent that controls yeast, an antiviral agent and an antiparasitic agent.

60. The method of claim 53, comprising (1) at least one vitamin; and (2) at least one additional therapeutic agent selected from the group of an antifungal agent, an antimicrobial agent, an antiviral agent, an anti-acne agent, an antipsoriasis agent, a keratolytic agent and an antiacne agent.

61. The method of claim 53, wherein the additional therapeutic agent comprises a combination of (1) a corticosteroid; and (2) and active agent selected from the group of an anti-infective agent, an antifungal agent, an antimicrobial agent, an antiviral agent, an anti-acne agent, a retinoid, a vitamin D, a vitamin D3 derivative, calcipotriol, calcitriol and antipsoriasis agent, a keratolytic agent, an anti-proliferative agent, an anti-cancer agent, a non-steroidal anti-inflammatory agent, an immunomodulator, an immunosuppressant and an anti-rosacea agent.

62. The method of claim 53, wherein the additional therapeutic agent comprises a combination of (1) a keratolytic agent; and (2) and active agent selected from the group of a corticosteroid, an anti-infective agent, an antifungal agent, an antimicrobial agent, an antiviral agent, an anti-acne agent, a retinoid, a vitamin D, a vitamin D3 derivative, calcipotriol, calcitriol and antipsoriasis agent, an anti-proliferative agent, an anti-cancer agent, a non-steroidal anti-inflammatory agent, an immunomodulator, an immunosuppressant and an anti-rosacea agent.

63. A method of treating a disorder or condition of mammalian subject, comprising:
administering to a target site a foam produced by release of a foamable vitamin and flavonoid composition from a pressurized canister containing a composition to which a vacuum was applied prior to filling with a hydrocarbon propellant, the composition comprising:
a. a vitamin and/or flavonoid; and
b. a substantially non-aqueous carrier, the carrier comprising
i. about 50% to about 98% of a polar solvent selected from the group consisting of polyols and polyethylene glycols (PEGs);
ii. 0% to about 48% of a secondary polar solvent;
iii. a surface-active agent or foam adjuvant;
iv. 0% to about 5% by weight of at least one polymeric agent;
v. 0% to about 5% of a conservation agent; and
vi. a propellant at a concentration of about 3% to about 25% by weight of the total composition;
wherein applying a vacuum and filling the canister containing the composition with hydrocarbon propellant contributes to stabilize the vitamin against degradation.

64. The method of claim 63, wherein the vitamin and or flavonoid or derivative thereof or combinations thereof are loaded into microsponges.

65. The method of claim 63, further comprising an additional therapeutic agent in addition to the vitamin, and or flavanoid a derivative thereof or combinations thereof.

66. The method of claim 63, wherein the target site is selected from the group consisting of the skin, a body cavity, a mucosal surface, the nose, the mouth, the eye, the ear canal, the respiratory system, the vagina and the rectum.

67. The method of claim 63, wherein the disorder is selected from the group consisting of dermatological pain, dermatological inflammation, acne, acne vulgaris, inflammatory acne, non-inflammatory acne, acne fulminans, nodular papulopustular acne, acne conglobata, dermatitis, bacterial skin infections, fungal skin infections, viral skin infections, parasitic skin infections, skin neoplasia, skin neoplasms, pruritis, cellulitis, acute lymphangitis, lymphadenitis, erysipelas, cutaneous abscesses, necrotizing subcutaneous infections, scalded skin syndrome, folliculitis, furuncles, hidradenitis suppurativa, carbuncles, paronychial infections, rashes, erythrasma, impetigo, ecthyma, yeast skin infections, warts, molluscum contagiosum, trauma or injury to the skin, post-operative or post-surgical skin conditions, scabies, pediculosis, creeping eruption, eczemas, psoriasis, pityriasis rosea, lichen planus, pityriasis rubra pilaris, edematous, erythema multiforme, erythema nodosum, grannuloma annulare, epidermal necrolysis, sunburn, photosensitivity, pemphigus, bullous pemphigoid, dermatitis herpetiformis, keratosis pilaris, callouses, corns, ichthyosis, skin ulcers, ischemic necrosis, miliaria, hyperhidrosis, moles, Kaposi's sarcoma, melanoma, malignant melanoma, basal cell carcinoma, squamous cell carcinoma, poison ivy, poison oak, contact dermatitis, atopic dermatitis, rosacea, purpura, moniliasis, candidiasis, baldness, alopecia, Behcet's syndrome, cholesteatoma, Dercum disease, ectodermal dysplasia, gustatory sweating, nail patella syndrome, lupus, hives, hair loss, Hailey-Hailey disease, chemical or thermal skin burns, scleroderma, aging skin, wrinkles, sun spots, necrotizing fasciitis, necrotizing myositis, gangrene, scarring, and vitiligo, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum; and wherein the active agent is suitable for treating said disorder.

68. The method of claim 63, wherein the at least one additional therapeutic agent is selected from the group consisting of active herbal extracts, acaricides, age spot and keratose removing agents, allergen, analgesics, local anesthetics, antiacne agents, antiallergic agents, antiaging agents, antibacterials, antibiotics, antiburn agents, anticancer agents, antidandruff agents, antidepressants, antidermatitis agents, antiedemics, antihistamines, antihelminths, antihyperkeratolyte agents, antiinflammatory agents, antiirritants, antilipemics, antimicrobials, antimycotics, antiproliferative agents, antioxidants, anti-wrinkle agents, antipruritics, antipsoriatic agents, antirosacea agents antiseborrheic agents, antiseptic, antiswelling agents, antiviral agents, anti-yeast agents, astringents, topical cardiovascular agents, chemotherapeutic agents, corticosteroids, dicarboxylic acids, disinfectants, fungicides, hair growth regulators, hormones, hydroxy acids, immunosuppressants, immunoregulating agents, insecticides, insect repellents, keratolytic agents, lactams, metals, metal oxides, mitocides, neuropeptides, non-steroidal anti-inflammatory agents, oxidizing agents, pediculicides, photodynamic therapy agents, retinoids, sanatives, scabicides, self tanning agents, skin whitening agents, asoconstrictors, vasodilators, wound healing agents and wart removers.

69. The method of claim 63, wherein the at least one active agent is selected from the group consisting of alclometasone dipropionate, amcinafel, amcinafide, amcinonide, beclomethasone, beclomethasone dipropionate, betamethsone, betamethasone benzoate, betamethasone dexamethasone-phosphate, dipropionate, betamethasone valerate, budesonide, chloroprednisone, chlorprednisone acetate, clescinolone, clobetasol, clobetasol propionate, clobetasol valerate, clobetasone, clobetasone butyrate, clocortelone, cortisone, cortodoxone, craposone butyrate, desonide, desoxymethasone, dexamethasone, desoxycorticosterone acetate, dichlorisone, diflorasone diacetate, diflucortolone valerate, difluorosone diacetate, diflurprednate, fluadrenolone, flucetonide, flucloronide, fluclorolone acetonide, flucortine butylesters, fludroxycortide, fludrocortisone, flumethasone, flumethasone pivalate, flumethasone pivalate, flunisolide, fluocinolone, fluocinolone acetonide, fluocinonide, fluocortin butyl, fluocortolone, fluorometholone, fluosinolone acetonide, fluperolone, fluprednidene acetate, fluprednisolone hydrocortamate, fluradrenolone, fluradrenolone acetonide, flurandrenolone, fluticasone, halcinonide, halobetasol, hydrocortisone, hydrocortisone acetate, hydrocortisone butyrate, hydrocortisone cyclopentylpropionate, hydrocortisone valerate, hydroxyltriamcinolone, medrysone, meprednisone, .alpha.-methyl dexamethasone, methylprednisolone, methylprednisolone acetate, mometasone furoate, paramethasone, prednisolone, prednisone, pregnenolone, progesterone, spironolactone, triamcinolone, triamcinolone acetonide and derivatives, esters and salts thereof.

70. The method of claim 63, wherein the at least one additional therapeutic agent is an anti-infective agent.

71. The method of claim 63, wherein the at least one anti-infective agent is selected from the group consisting of an antibiotic agent, an antibacterial agent, an antifungal agent, an agent that controls yeast, an antiviral agent and an antiparasitic agent.

72. The method of claim 63, comprising (1) at least one vitamin; and (2) at least one additional therapeutic agent selected from the group of an antifungal agent, an antimicrobial agent, an antiviral agent, an anti-acne agent, an antipsoriasis agent, a keratolytic agent and an antiacne agent.

73. The method of claim 63, wherein the additional therapeutic agent comprises a combination of (1) a corticosteroid; and (2) and active agent selected from the group of an anti-infective agent, an antifungal agent, an antimicrobial agent, an antiviral agent, an anti-acne agent, a retinoid, a vitamin D, a vitamin D3 derivative, calcipotriol, calcitriol and antipsoriasis agent, a keratolytic agent, an anti-proliferative agent, an anti-cancer agent, a non-steroidal anti-inflammatory agent, an immunomodulator, an immunosuppressant and an anti-rosacea agent.

74. The method of claim 63, wherein the additional therapeutic agent comprises a combination of (1) a keratolytic agent; and (2) and active agent selected from the group of a corticosteroid, an anti-infective agent, an antifungal agent, an antimicrobial agent, an antiviral agent, an anti-acne agent, a retinoid, a vitamin D, a vitamin D3 derivative, calcipotriol, calcitriol and antipsoriasis agent, an anti-proliferative agent, an anti-cancer agent, a non-steroidal anti-inflammatory agent, an immunomodulator, an immunosuppressant and an anti-rosacea agent.

75. A non-aqueous or substantially non aqueous foamable composition, comprising:
ascorbic acid in an amount of up to about 7 wt %;
about 50% to about 98% of a hygroscopic solvent comprising a mixture of at least one polyol and PEG;
at least one of a surfactant and a foam adjuvant selected from the group consisting of fatty alcohol, a fatty acid and a hydroxyl fatty acid or liquid wax; and a hydrocarbon propellant at a concentration of about 3% to about 25% by weight of the total composition,
wherein said foamable carrier reduces the destabilizing effect of water on the vitamin, and
wherein the composition is contained in a pressurized container, is substantially flowable and provides a foam upon release.
